SEQUENCE LISTING (9 sequences)

SEQ ID NO: 1 - protein, 406 aa, Artificial Sequence
  synthetic construct
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 2 - protein, 398 aa, Artificial Sequence
  synthetic construct
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 3 - protein, 995 aa, Artificial Sequence
  synthetic construct
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 4 - protein, 397 aa, Artificial Sequence
  synthetic construct
HHHHHHWSHPQFEKGGGSGGGSGGSSAWSHPQFEKFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 5 - protein, 364 aa, Artificial Sequence
  synthetic construct
HHHHHHHHHHRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 6 - protein, 969 aa, Artificial Sequence
  synthetic construct
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKKHHHHHHHHHH

SEQ ID NO: 7 - protein, 362 aa, Artificial Sequence
  synthetic construct
FQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 8 - protein, 354 aa, Artificial Sequence
  synthetic construct
RDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 9 - protein, 959 aa, Artificial Sequence
  synthetic construct
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

(19) United States
(12) Patent Application Publication
(10) Pub. No.: US 2023/0372528 A1
(43) Pub. Date: Nov. 23, 2023

(54) GLYCOCONJUGATES

(71) Applicants: UNIVERSITY OF GEORGIA RESEARCH FOUNDATION, INC., Athens, GA (US); MEDIMMUNE LIMITED, Cambridge, Cambridgeshire (GB)

(72) Inventors: Gerardus Josephus BOONS, Athens, GA (US); Xiuru LI, Athens, GA (US); Patricius Hendrikus Cornelis VAN BERKEL, Epalinges (CH)

(21) Appl. No.: 18/248,335

(22) PCT Filed: Oct. 14, 2021
(86) PCT No.: PCT/EP2021/078536

§ 371 Date: Apr. 7, 2023

Related U.S. Application Data
(60) Provisional application No. 63/092,641, filed on Oct. 16, 2020.

(51) Int. Cl. A61K 47/68; A61K 47/54; A61P 35/00

(52) U.S. Cl. CPC A61K 47/6889; A61K 47/549; A61K 47/6855; A61K 47/6803; A61P 35/00

(57) ABSTRACT

This disclosure relates to glycoconjugates comprising glycosylated cell-binding agents conjugated to pyrrolobenzodiazepine (PBD) payloads. Glycoconjugates of particular interest include conjugates where the cell-binding agent is an antibody and the payload comprises a cytotoxic pyrrolobenzodiazepine (PBD) moiety, with the PBD moiety conjugated to the antibody via an oligosaccharide linker. The disclosure also relates to methods for preparing the glycoconjugates, along with methods for their use.

### EARLIER APPLICATION

This application claims priority from U.S. provisional application No. 63/092,641, filed 16 Oct. 2020. The priority application is hereby incorporated by reference in its entirety and for any and all purposes as if fully set forth herein.

### FIELD

This disclosure relates to glycoconjugates comprising glycosylated cell-binding agents conjugated to pyrrolobenzodiazepine (PBD) payloads. Glycoconjugates of particular interest include conjugates where the cell-binding agent is an antibody and the payload comprises a cytotoxic pyrrolobenzodiazepine (PBD) moiety, with the PBD moiety conjugated to the antibody via an oligosaccharide linker. The disclosure also relates to methods for preparing the glycoconjugates, along with methods for their use.

### BACKGROUND

Antibody Therapy

Antibody therapy has been established for the targeted treatment of subjects with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumour cells in the treatment of cancer, targets delivery of the drug moiety to tumours, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

The field has been advanced by the emergence of new classes of potent toxins, such as taxanes, calicheamycins, maytansins, pyrrolobenzodiazepines, duocarmycins and auristatins. The low nanomolar to picomolar toxicity of these substances has provided significant advantages over earlier generations of toxins. Another technological advance involves the use of optimized linkers that are hydrolysable in the cytoplasm, resistant or susceptible to proteases, or resistant to multi-drug resistance efflux pumps that are associated with highly cytotoxic drugs.

A common mode for preparing ADCs is the conjugation of the payload (eg. A drug-linker molecule) to the side chain of antibody amino acid lysine or cysteine. The kinetics of lysine addition means conjugation at this residue takes place preferentially at lysine side chains with high steric accessibility and low pKa, making the site-specificity of the reaction difficult to control. More site specificity is offered by conjugating to cysteines, since there are typically no free cysteine sulfhydryl groups present in a wild-type antibody under normal conditions. This allows for methods where free sulfhydryl groups can be introduced into the antibody molecule by, for example, selective reduction of existing cysteine of the introduction of additional cysteines through protein engineering. In both case, payloads can be effectively conjugated to the freed sulfhydryl groups using, for example, electrophilic alkylation based on maleimide addition. This method allows for efficient and site-selective generation of conjugates. However, given the benefits of high product homogeneity and conjugates with high resistance to off-target release of payload, research has continued to identify conjugation methods offering further improvements over sulfhydryl alkylation.

Azide, Hydroxylamine, and Hydrazine Conjugations

One alternative conjugation technology makes use of azide chemistry (N₃ groups, also referred to as azido groups). In particular, azide groups are able to undergo selective cycloaddition with terminal alkynes (copper-catalyzed) or with cyclic alkynes (copper free, with the reaction promoted by ring strain). The triazoles resulting from reaction with alkynes are particularly resistant to hydrolysis and other degradation pathways. This reaction has been shown to have utility in the production of ADCs (see, for example, WO2014/065661, and Li et al., Angew Chem Int Ed Engl., 2014, Jul. 7; 53(28):7179-82). Potential use in ADC production has also been discussed for ketones plus either hydroxylamines or hydrazines (see WO2014/065661).

Glycoconjugates

A number of strategies exist for introducing the above functional groups into conjugate precursors have been discussed. One strategy that has been demonstrated to yield safe and effective ADCs involves conjugation of the payload to the glycan moiety of a glycosylated cell-binding agent, such as an antibody (see, for example, WO/2018/146189).

Conjugation via glycans is a potentially versatile strategy for ADC generation, as—for example—all IgG antibodies expressed in mammalian or yeast cell cultures bear a N-linked glycan moiety on the Fc portion of each heavy chain. However, this methodology presents a number of challenges. For example, glycans are typically present as a complex mixture of isoforms, which may contain different levels of galactosylation (G0, G1, G2) and fucosylation (G0F, G1F, G2F) which may in turn lead to undesirable heterogeneity in conjugation stoichiometry. Accordingly, existing methods often employ one or more ‘glycan remodelling’ steps in which enzymes are used to trim and/or add carbohydrate moieties in order to homogenise the glycan structures as much as possible prior to conjugation with the payload (see WO 2007/133855, WO2014/065661, and Li et al., Angew Chem Int Ed Engl., 2014, Jul. 7; 53(28):7179-82). However, the huge variety of possible sugar moieties, linkages, branching, chain length, and modifying enzymes available mean there is a vast genus of possible final structures of the glycan moiety. The final size and structure of the glycan influences many key properties of the final glycoconjugate (eg. drug-antibody-ratio, conjugate hydrophilicity, conjugate hydrodynamic etc.) many of which cannot be reliably predicted a priori. Accordingly, research into advantageous glycan configurations is ongoing.

Once the glycoprotein has been remodelled there are several possible strategies for conjugation to a payload. For example, numerous methods have been described involving the condensation of the homogenised glycoprotein with singly or multiply azide or alkyne-functionalised saccharides to yield activated glycoprotein intermediates which are then conjugated to payloads using the above-described chemistry (for additional detail see discussion in, for example, WO2014/06566, Li et al., Angew Chem Int Ed Engl., 2014, Jul. 7; 53(28):7179-82, and the references cited therein).

The above methods have been demonstrated to yield glycoconjugates having in vivo anticancer efficacy (see, for example, WO/2018/146189). Nonetheless, research to further improve the properties of such glycoconjugates across a range of cell-binding agents and payloads is ongoing.

### SUMMARY

The present authors have investigated the properties of a range of oligosaccharide structures were investigated with the aim of identifying oligosaccharide structures that both (1) allowed for advantageous glycoconjugate properties, and (2) were readily amenable to commercial-scale manufacture.

During their investigation, the authors discovered that glycoconjugates having a relatively short trisaccharide moiety of -[GlcNAc]-[Gal]-[Sia]- between cell-binding agent and payload had a range of advantageous properties. For example, compared to otherwise similar glycoconjugates having larger glycan moieties this class of glycoconjugates was unexpectedly found to: have higher hydrophilicity and solubility; significantly faster kinetics of conjugation; significantly more efficacious in vivo (despite similar in vitro activity); better control/consistency of achieving Drug-to-Antibody Ratio (DAR)=2; and significantly improved tolerability of treatment by subjects. Without wishing to be bound by theory, the present authors believe that these properties arise in part due to the presence and location of the negatively charged sialic residue. For some payloads this was found to be associated with improved glycoconjugate efficacy as compared to uncharged sugar moieties in the same position.

The present authors further determined that the advantageous -[GlcNAc]-[Gal]-[Sia]- glycoconjugates could be manufactured using readily-available enzyme catalysts. In particular, it was unexpectedly found that the wild-type human β4GalT1 galactosyl-transferase was able to efficiently transfer a galactose residue onto a α1-6 fucosylated GlcNAc residue, despite that reaction not occurring in the natural system in which this enzyme is found. The galactosylated oligosaccharide resulting from that reaction was also readily susceptible to the addition of an alkynyl or azido-modified sialic acid by the wild-type ST6Gal1 sialyltransferase.

Accordingly, in a first aspect the present disclosure provides a glycoconjugate having the formula:

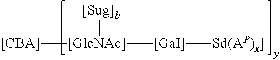

wherein:
CBA is a Cell Binding Agent;
GlcNAc is a N-acetyl-glucosamine moiety;
Sug is a sugar moiety, wherein b=1 or 0;
Gal is a galactose moiety;
Sd(A^{P})ₓ is a sugar derivative comprising x conjugated payloads A^{P}, wherein x=1, 2, 3, or 4; and
wherein y=1 to 20.

In some cases b=0. In other cases b=1.

Preferably the saccharide molecules and moieties such as “GlcNAc”, “Sug”, and “Gal” described herein are ‘D’ enantiomers.

In some cases Sd(A^{P})x is a sialic acid derivative, such as a sialic acid derivative having the formula:

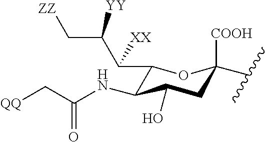

wherein:
QQ is hydrogen or a conjugated payload;
ZZ is hydroxyl or a conjugated payload;
YY is hydroxyl or a conjugated payload; and/or
XX is hydroxyl or a conjugated payload;

and wherein at least one of QQ, ZZ, YY, and XX is a conjugated payload.

In some cases the glycoconjugate has the formula:

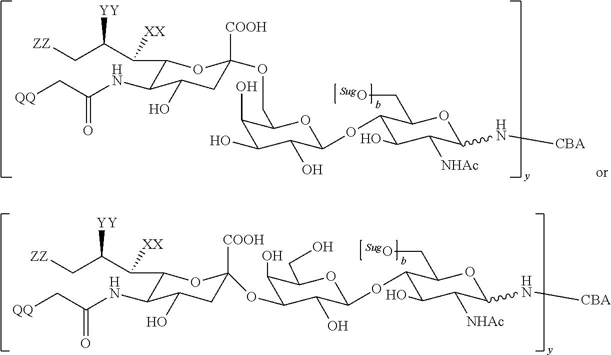

wherein:
QQ is hydrogen or a conjugated payload;
ZZ is hydroxyl or a conjugated payload;
YY is hydroxyl or a conjugated payload; and/or
XX is hydroxyl or a conjugated payload;

and wherein at least one of QQ, ZZ, YY, and XX is a conjugated payload.

Typically Sug is linked to the GlcNAc at the GlcNAc C6, preferably in an α1-6 configuration. In some cases Sug is a fucose moiety, such as a fucose moiety having the structure:

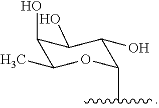 [SMILES: *C1OC(C)C(O)C(O)C1O]

In some case the glycoconjugate has a conjugated payload at position QQ. In some cases the glycoconjugate has a conjugated payload at position QQ. In some cases the glycoconjugate has a conjugated payload at position ZZ. In some cases the glycoconjugate has a conjugated payload at each of positions QQ and ZZ. QQ and ZZ may be the same or different.

In some x=1. In some cases x=2. In some cases y=1, 2, 3, or 4. In some cases y=2. In some cases y=1 to 2, 1 to 3, 2 to 4, 3-6 or 4-8.

In some cases the GlcNAc moiety is linked to the cell-binding agent via the GlcNAc C1 carbon. In some cases the CBA-N-GlcNAc linkage is in the beta anomeric configuration. In cases where the cell-binding agent is a peptide, the GlcNAc moiety may be α-linked to an asparagine residue in the peptide backbone. In cases where the cell-binding agent is an antibody, the GlcNAc is preferably conjugated to the antibody at the asparagine 297 (Asn297) residue according to the EU index as set forth in Kabat.

The CBA may be a protein, such as a therapeutic protein, an antibody, and/or a Fc fusion protein. An antibody may be monoclonal and/or of the IgG isotype, such as the IgG1 subclass. An antibody may be an intact antibody. A Fc fusion protein may comprise a Fc domain is of the IgG isotype, such as the IgG1 subclass.

The CBA may specifically bind a target antigen selected from the group comprising of: BMPR1B, E16, STEAP1, 0772P, MPF, Napi3b, Sema 5b, PSCA hIg, ETB, MSG783, STEAP2, TrpM4, CRIPTO, CD21, CD79b, FcRH2, HER2, NCA, MDP, IL20R-alpha, Brevican, EphB2R, ASLG659, PSCA, GEDA, BAFF-R, CD22, CD79a, CXCR5, HLA-DOB, P2X5, CD72, LY64, FcRH1, IRTA2, TENB2, PSMA, SST, ITGAV, ITGB6, CEACAM5, MET, MUC1, CA9, EGFRvIII, CD33, CD19, IL2RA, AXL, CD30, BCMA, CT Ags, CD174, CLEC14A, GRP78-HSPA5, CD70, Stem Cell specific antigens, ASG-5, ENPP3, PRR4, GCC-GUCY2C, Liv-1-SLC39A6, 5T4, CD56-NCMA1, CanAg, FOLR1, GPNMB, TIM-1-HAVCR1, RG-1, B7-H4-VTCN1, PTK7, CD37, CD138, CD74, Claudins, EGFR, Her3, RON-MST1R, EPHA2, CD20-MS4A1, Tenascin C-TNC, FAP, DKK-1, CD52, CS1-SLAMF7, Endoglin, Annexin A1, V-CAM (CD106), DLK-1, KAAG1, IL13RA2, Endosialin, CD48, LRRC15, SLAMF6, and PLAC1.

The payload is a ‘PBD payload’. That is, the payload is, comprises, or releases upon metabolism a PBD compound, as defined in the section herein entitled “PBD compound”.

In some cases the payload has a linker moiety linking the CBA and the remainder of the payload. The linker may comprise an ionizable group such as: an acidic group/moiety, such as, for example, —CO₂H, —NHSO₂NH₂, —NHSO₂NHR where R is an alkyl moiety, —SO₃H, sialic acid, glutamic acid, a glutamic acid sidechain, aspartic acid, an aspartic acid sidechain, and the like, and salts or ionic groups/moieties formed therefrom; or a basic group/moiety, such as, for example, an amine group/moiety (e.g., a primary amine group, a secondary amine moiety, or a tertiary amine moiety), guanidinium group, and the like, and salts or ionic groups/moieties formed therefrom; with the proviso that no carbonyl is adjacent (e.g., alpha) to a —NHSO₂NH— moiety or —NHSO₂NH₂ group. In some cases the conjugated payload is a conjugated PBD drug-linker payload as defined in the section herein titled “PBD drug-linker embodiments”.

In a third aspect, the present disclosure provides a method for the preparation of the glycoconjugate of the first aspect, the method comprising the steps of:
(i) providing a Sd(A^{F})ₓ acceptor having the formula:

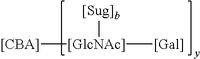

wherein CBA, GlcNAc, Sug, Gal, and y are defined as above; and
(ii) contacting the Sd(A^{F})ₓ acceptor with a compound of the formula Sd(A^{F})ₓ-P* in the presence of a glycosyltransferase to produce a glycosylated cell-binding agent, wherein:
Sd(A^{F})ₓ is as defined as for Sd(A^{P})ₓ above, except that “A^{F}” represents a functional group A^{F} instead of a conjugated payload A^{P}; and
P* is a nucleoside phosphate moiety; and
(iii) reacting the glycosylated cell-binding agent of (ii) with a compound of the formula payload-G^{L}, wherein the payload is as above and G^{L} is linker group reactive with the functional group A^{F} of the glycosylated cell-binding agent.

In some cases functional group A^{F} is an azido group. In some cases a functional group A^{F} is an alkynyl group.

In some case G^{L} is selected from the group defined herein.

In some cases, the nucleoside phosphate moiety is one of adenosine, guanosine, uridine, cytidine, or thymidine; such as, a nucleoside phosphate moiety selected from the group consisting of: UDP, GDP, TDP, CDP, and CMP.

In some cases, the Sd(A^{F})ₓ acceptor above is provided by a process comprising the steps of:
a) providing a Gal acceptor having the formula:

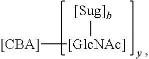

wherein CBA, GlcNAc, Sug, b, and y are defined as described elsewhere herein for glycosylated cell-binding agents; and
b) contacting the Gal acceptor with a compound of the formula Gal-P* in the presence of a galactosyltransferase, wherein Gal and P* are as defined above;
   optionally wherein,
c) the Gal acceptor is produced by contacting with a glycosidase a oligoglycosylated cell-binding agent having the formula:

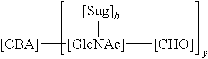

wherein CHO is a carbohydrate moiety.

In some cases the Sd(A^{F})ₓ acceptor has only one terminal galactose moiety. In some cases the Sd(A^{F})ₓ acceptor has only two terminal galactose moieties. In some cases the Sd(A^{F})ₓ acceptor has only three terminal galactose moieties. In some cases the Sd(A^{F})ₓ acceptor has only four terminal galactose moieties.

The glycosyltransferase may be a sialyltransferase, such as human beta-galactoside alpha-2,6-sialyltransferase 1 (ST6Gal1). In some cases the sialyltransferase has the sequence set out in SEQ ID NO. 1, 4, or 7. The glycosidase may be an endoglycosidase, such as Endo S as disclosed in Collin, M. and Olsén, A. (2001). The EMBO Journal. 20, 3046-3055. In some cases the endoglycosidase has the sequence set out in SEQ ID NO. 3, 6, or 9. The galactosyltransferase may be human beta-1,4-galactosyltransferase 1 (B4GalT1). In some cases the galactosyltransferase has the sequence set out in SEQ ID NO. 2, 5, or 8.

In a fourth aspect the present disclosure provides the use of the glycosylated cell-binding agent of the third aspect in the production of the glycoconjugate according to the first aspect.

In a fifth aspect, the present disclosure provides a glycoconjugate of any the first aspect for use in a method of treatment. In some cases the method of treatment is a method of treating a proliferative disorder, such as cancer. The cancer may be selected from the group consisting of: histocytoma, glioma, astrocytoma, neuroblastoma, osteoma, lung cancer, gastrointestinal cancer, bowel cancer, colon cancer, breast carcinoma, ovarian carcinoma, prostate cancer, testicular cancer, liver cancer, kidney cancer, bladder cancer, pancreas cancer, renal cancer, brain cancer, sarcoma, liposarcoma, osteosarcoma, Kaposi's sarcoma, melanoma, lymphomas, myeloma, and leukemias.

Also provided herein are methods of treating a proliferative disorder (optionally as defined in the fifth aspect), the method comprising administering an effective amount of a glycoconjugate of the first aspect. Uses of a glycoconjugate of the first aspect in the manufacture of a medicament for the treatment of a proliferative disorder (optionally as defined in the fifth aspect) are also disclosed herein.

### DETAILED DESCRIPTION

Glycoconjugates

The present disclosure concerns glycoconjugates in which a cell-binding agent is conjugated to a payload via a short glycan moiety on the heavy chain of the antibody. In particular, the present disclosure relates to glycoconjugates wherein the glycan moiety is the trisaccharide -[GlcNAc]-[Gal]-[Sia]-, wherein the GlcNAc residue (a term used interchangeably herein with GlcNac moiety) is optionally branched with a sugar residue such as a fucose residue.

Embodiments of particular interest include glycoconjugates wherein the cell-binding agent is an antibody or a FC fusion protein; for example, an IgG antibody or a Fc fusion protein comprising an IgG Fc domain. Glycoconjugates wherein the payload comprises a cytotoxic pyrrolobenzodiazepine (PBD) compound are also of particular interest.

Accordingly, in a first aspect the present disclosure provides a glycoconjugate having the formula:

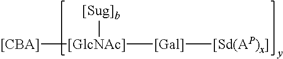

wherein:
CBA is a Cell Binding Agent;
GlcNAc is a N-acetyl-glucosamine moiety;
Sug is a sugar moiety, wherein b=1 or 0;
Gal is a galactose moiety;
Sd(A^{P})ₓ is a sugar derivative comprising x conjugated payloads A^{P}, wherein x=1, 2, 3, or 4; and
wherein y=1 to 20.

In some embodiments b=0. In some embodiments b=1.

In some embodiments, the average number of payloads per CBA (that is “y”) is in the range 1 to 4. In some embodiments the range is selected from 1 to 2, 1 to 3, 2 to 4, 3-6 or 4-8.

The GlcNAc moiety is typically conjugated to the CBA via the GlcNAc C1 carbon. Preferably the CBA-N-GlcNAc linkage is in the beta anomeric configuration.

As described elsewhere herein, the glycoconjugates are typically produced and/or modified using enzyme catalysed processes. Accordingly, the saccharide molecules and moieties described herein (eg. “GlcNAc”, “Sug”, “Gal”) typically have the properties and configuration that allows for their efficient use by the enzyme catalysts. Preferably the saccharide molecules and moieties such as “GlcNAc”, “Sug”, and “Gal” described herein are ‘D’ enantiomers.

In some preferred embodiments, the glycoconjugate has the formula:

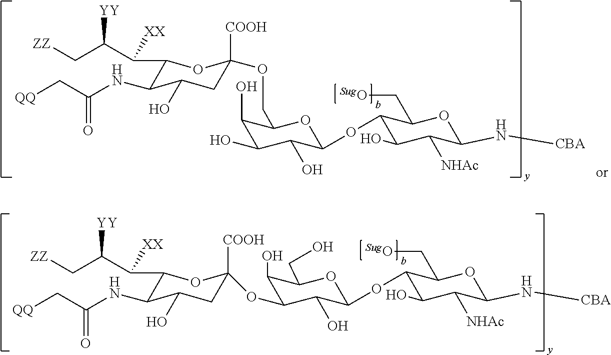

wherein:
QQ is hydrogen or a conjugated payload;
ZZ is hydroxyl or a conjugated payload;
YY is hydroxyl or a conjugated payload; and/or
XX is hydroxyl or a conjugated payload;

and wherein at least one of QQ, ZZ, YY, and XX is a conjugated payload

In preferred embodiments, the GlcNAc moiety can be bound to the CBA with an α-N-glycosidic linkage:

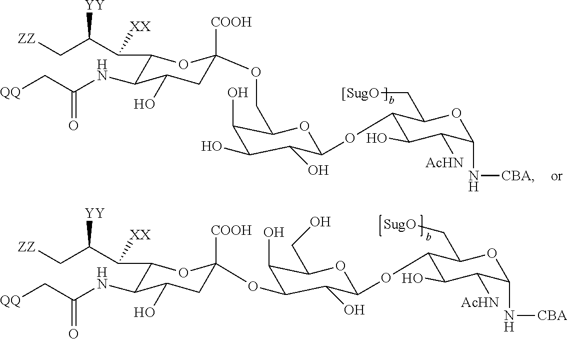

In embodiments where the cell-binding agent is a peptide or polypeptide (such as an antibody), or comprises a peptide or polypeptide portion, the oligosaccharide may be conjugated to the antibody through an asparagine side chain via an α-N-glycosidic bond:

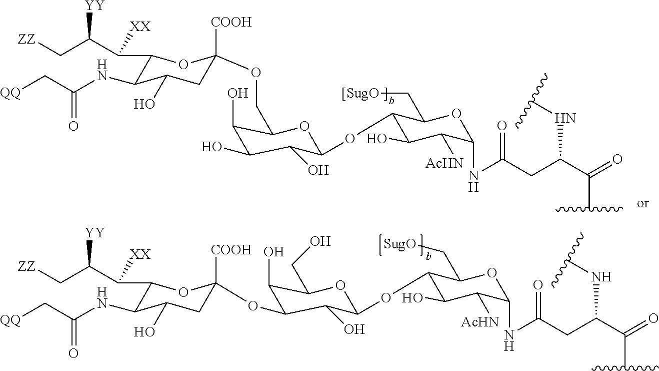

In preferred embodiments the CBA is an antibody. In some cases the GlcNAc moiety is conjugated to the antibody at the asparagine 297 (Asn297) residue according to the EU index as set forth in Kabat. In embodiments wherein y is 1, the GlcNAc moiety may be conjugated to one of the Asn297 residues in the Fc domain. In embodiments wherein y is 2, a GlcNAc moiety is conjugated to each of the two Asn297 residues in the Fc domain. In embodiments where the antibody has been modified—for example by chain elongation or truncation—the GlcNAc moiety may be conjugated to the asparagine residue corresponding to Asn297 of the unmodified antibody.

In some preferred embodiments the glycoconjugate has a conjugated payload at position QQ. In some of these embodiments all of XX, YY, and ZZ are hydroxyl.

In some other preferred embodiments the glycoconjugate has a conjugated payload at position ZZ. In some of these embodiments QQ is hydrogen and XX and YY are hydroxyl.

In some embodiments the glycoconjugate has a conjugated payload at each of positions QQ and ZZ. QQ and ZZ may be the same or different. In some of these embodiments XX and YY are hydroxyl.

Provided herein are highly homogenous glycoconjugates, meaning that each individual CBA has the same glycan structures conjugated to the CBA (ie. are the same glycoform). In some embodiments at least 75%, such as at least 80%, at least 85%, at least 90%, at least 95%, at least 97.5%, or at least 99% of the individual CBA molecules in the composition bear an identical glycan structure (ie. are the same glycoform). For embodiments where the CBA is an antibody and in which the oligosaccharide is glycosylated to Asn297, at least 75%, at least 80%, at least 85%, at least 90%, at least 95%, at least 97.5%, or at least 99% of the antibodies may bear an identical glycan structure at Asn297.

Payload Loading

The payload loading (p) is the average number of payloads per CBA, e.g. antibody.

The average number of payloads per CBA in preparations from conjugation reactions may be characterized by conventional means such as UV, reverse phase HPLC, HIC, mass spectroscopy, ELISA assay, and electrophoresis. The quantitative distribution of CBA in terms of p may also be determined. By ELISA, the averaged value of p in a particular preparation of CBA may be determined (Hamblett et al (2004) Clin. Cancer Res. 10:7063-7070; Sanderson et al (2005) Clin. Cancer Res. 11:843-852). However, the distribution of p values is not discernible by the CBA-antigen binding and detection limitation of ELISA. Also, ELISA assay for detection of glycoconjugates does not determine where the payloads are attached to the CBA, such as the heavy chain or light chain fragments of antibodies, or the particular amino acid residues. In some instances, separation, purification, and characterization of homogeneous CBA where p is a certain value from CBA with other payload loadings may be achieved by means such as reverse phase HPLC or electrophoresis. Such techniques are also applicable to other types of conjugates.

For the present glycoconjugates, p is limited by the number of attachment sites on the CBA, eg. the number of azide groups. For example, the CBA may have only one or two azide groups to which the payload may be attached.

Typically, fewer than the theoretical maximum of payloads are conjugated to a CBA during a conjugation reaction. The loading ratio of a CBA may be controlled in several different manners, including: (i) limiting the molar excess of payload intermediate or linker reagent relative to CBA, and (ii) limiting the conjugation reaction time or temperature.

Where more than one nucleophilic or electrophilic group of the CBA reacts with a payload intermediate, or linker reagent followed by payload reagent, then the resulting product is a mixture of glycoconjugates with a distribution of payloads attached to a CBA, e.g. 1, 2, 3, etc. Liquid chromatography methods such as polymeric reverse phase (PLRP) and hydrophobic interaction (HIC) may separate compounds in the mixture by p value. Preparations of CBA with a single drug loading value (p) may be isolated, however, these single loading value CBAs may still be heterogeneous mixtures because the payloads may be attached, via the linker, at different sites on the CBA.

Thus the glycoconjugate compositions described herein include mixtures of glycoconjugate compounds where the CBA has one or more payloads and where the payloads may be attached to the antibody at various conjugation sites.

In one embodiment, the average number of payloads per CBA is in the range 1 to 4. In some embodiments the range is selected from 1 to 2 or 2 to 4.

In some embodiments, there are approximately 2 payloads per CBA. In some embodiments there are approximately 4 payloads per CBA.

In some embodiments, each glycan of the CBA is conjugated to one payload (ie. x=1). In some embodiments, each glycan of the CBA is conjugated to two payloads (ie. x=2).

In some embodiments, the CBA has one glycan-payload moiety (ie. y=1). In some embodiments, the CBA has two glycan-payload moieties (ie. y=2). In come embodiments, the CBA has 1 to 4 glycan-payload moieties (ie. y=1 to 4).

In some preferred embodiments, the CBA is an intact antibody having exactly two glycan-payload moieties (ie. y=2). In some of those embodiments, each glycan-payload moiety has exactly one payload (x=1).

Cell Binding Agents (CBA)

A cell binding agent may be of any kind, and include peptides and non-peptides. Suitable agents include antibodies or a fragment of an antibody that contains at least one binding site, Fc fusion proteins, lymphokines, hormones, hormone mimetics, vitamins, growth factors, nutrient-transport molecules, or any other cell binding molecule or substance.

Preferred CBAs include proteins and peptides, including therapeutic proteins or peptides. Antibodies and Fc fusion proteins are particularly preferred CBAs.

The CBAs typically bind cells through specific binding of one or more antigens expressed by the target cell. These antigens are herein termed ‘target antigens’ and are typically expressed on the surface of the target cell.

As used herein to describe cell-binding agents, “specifically binds [target antigen]” means that the CBA binds the antigen with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the CBA binds the antigen with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The CBAs of the disclosure typically bind the antigen with a high affinity. For example, in some embodiments the CBA can bind the antigen with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴.

Target Antigens

In some aspects, the specifically bound target antigen is selected from the group consisting of:

(1) BMPR1B (Bone Morphogenetic Protein Receptor-Type IB)

Nucleotide

Genbank accession no. NM_001203

Genbank version no. NM_001203.2 GI:169790809

Genbank record update date: Sep. 23, 2012 02:06 PM

Polypeptide

Genbank accession no. NP_001194

Genbank version no. NP_001194.1 GI:4502431

Genbank record update date: Sep. 23, 2012 02:06 PM

### Cross-References

ten Dijke, P., et al Science 264 (5155): 101-104 (1994), Oncogene 14 10 (11):1377-1382 (1997)); WO2004/063362 (Claim 2); WO2003/042661 (Claim 12);
US2003/134790-A1 (Page 38-39); WO2002/102235 (Claim 13; Page 296); WO2003/055443 (Page 91-92); WO2002/99122 (Example 2; Page 528-530); WO2003/029421 (Claim 6);
WO2003/024392 (Claim 2; FIG. 112); WO2002/98358 (Claim 1; Page 183); WO2002/54940 (Page 100-101); WO2002/59377 (Page 349-350); WO2002/30268 (Claim 27; Page 376);
15 WO2001/48204 (Example; FIG. 4); NP_001194 bone morphogenetic protein receptor, type IB/pid=NP_001194.1; MIM:603248; AY065994

(2) E16 (LAT1, SLC7A5)

Nucleotide

Genbank accession no. NM_003486

Genbank version no. NM_003486.5 GI:71979931

Genbank record update date: Jun. 27, 2012 12:06 PM

Polypeptide

Genbank accession no. NP_003477

Genbank version no. NP_003477.4 GI:71979932

Genbank record update date: Jun. 27, 2012 12:06 PM

### Cross References

Biochem. Biophys. Res. Commun. 255 (2), 283-288 (1999), Nature 395 (6699):288-291 (1998), Gaugitsch, H. W., et 20 al (1992) J. Biol. Chem. 267 (16):11267-11273); WO2004/048938 (Example 2); WO2004/032842 (Example IV); WO2003/042661 (Claim 12); WO2003/016475 (Claim 1); WO2002/78524 (Example 2); WO2002/99074 (Claim 19; Page 127-129); WO2002/86443 (Claim 27; Pages 222, 393); WO2003/003906 (Claim 10; Page 293); WO2002/64798 (Claim 33; Page 93-95); WO2000/14228 (Claim 5; Page 133-136); US2003/224454 (FIG. 3);
25 WO2003/025138 (Claim 12; Page 150); NP_003477 solute carrier family 7 (cationic amino acid transporter, y+system), member 5/pid=NP_003477.3—Homo sapiens;
MIM:600182; NM_015923.

(3) STEAP1 (Six Transmembrane Epithelial Antigen of Prostate)

Nucleotide

Genbank accession no. NM_012449

Genbank version no. NM_012449.2 GI:22027487

Genbank record update date: Sep. 9, 2012 02:57 PM

Polypeptide

Genbank accession no. NP_036581

Genbank version no. NP_036581.1 GI:9558759

Genbank record update date: Sep. 9, 2012 02:57 PM

### Cross References

Cancer Res. 61 (15), 5857-5860 (2001), Hubert, R. S., et al (1999) Proc. Natl. Acad. Sci. U.S.A. 96 (25):14523-14528); WO2004/065577 (Claim 6); WO2004/027049 (FIG. 1L); EP1394274 (Example 11); WO2004/016225 (Claim 2); WO2003/042661 (Claim 12); US2003/157089 (Example 5); US2003/185830 (Example 5); US2003/064397 (FIG. 2); WO2002/89747 (Example 5; Page 618-619); WO2003/022995 (Example 9; FIG. 13A,
35 Example 53; Page 173, Example 2; FIG. 2A); six transmembrane epithelial antigen of the prostate; MIM:604415.

(4) 0772P (CA125, MUC16)

Nucleotide

Genbank accession no. AF361486

Genbank version no. AF361486.3 GI:34501466

Genbank record update date: Mar. 11, 2010 07:56 AM

Polypeptide

Genbank accession no. AAK74120

Genbank version no. AAK74120.3 GI:34501467

Genbank record update date: Mar. 11, 2010 07:56 AM

### Cross References

J. Biol. Chem. 276 (29):27371-27375 (2001)); WO2004/045553 (Claim 14); WO2002/92836 (Claim 6; FIG. 12); WO2002/83866 (Claim 15; Page 116-121); US2003/124140 (Example 16); GI:34501467;

(5) MPF (MPF, MSLN, SMR, Megakaryocyte Potentiating Factor, Mesothelin)

Nucleotide

Genbank accession no. NM_005823

Genbank version no. NM_005823.5 GI:293651528

Genbank record update date: Sep. 2, 2012 01:47 PM

Polypeptide

Genbank accession no. NP_005814

Genbank version no. NP_005814.2 GI:53988378

Genbank record update date: Sep. 2, 2012 01:47 PM

### Cross References

Yamaguchi, N., et al Biol. Chem. 269 (2), 805-808 (1994), Proc. Natl. Acad. Sci. U.S.A. 96 (20):11531-11536 (1999), Proc. Natl. Acad. Sci. U.S.A. 93 10 (1):136-140 (1996), J. Biol. Chem. 270 (37):21984-21990 (1995)); WO2003/101283 (Claim 14); (WO2002/102235 (Claim 13; Page 287-288); WO2002/101075 (Claim 4; Page 308-309); WO2002/71928 (Page 320-321); WO94/10312 (Page 52-57); IM:601051.

(6) Napi3b (NAPI-3B, NPTIIb, SLC34A2, Solute Carrier Family 34 (Sodium Phosphate), Member 2, Type II Sodium-Dependent Phosphate Transporter 3b)

Nucleotide

Genbank accession no. NM_006424

Genbank version no. NM_006424.2 GI:110611905

Genbank record update date: Jul. 22, 2012 03:39 PM

Polypeptide

Genbank accession no. NP_006415

Genbank version no. NP_006415.2 GI:110611906

Genbank record update date: Jul. 22, 2012 03:39 PM

### Cross References

J. Biol. Chem. 277 (22):19665-19672 (2002), Genomics 62 (2):281-284 (1999), Feild, J. A., et al (1999) Biochem. Biophys. Res. Commun. 258 (3):578-582); WO2004/022778 (Claim 2); EP1394274 (Example 11); WO2002/102235 (Claim 13; Page 20 326); EP0875569 (Claim 1; Page 17-19); WO2001/57188 (Claim 20; Page 329); WO2004/032842 (Example IV); WO2001/75177 (Claim 24; Page 139-140); MIM:604217.

(7) Sema 5b (FLJ10372, KIAA1445, Mm.42015, SEMA5B, SEMAG, Semaphorin 5b H log, 25 Sema Domain, Seven Thrombospondin Repeats (Type 1 and Type 1-Like), Transmembrane Domain (TM) and Short Cytoplasmic Domain, (Semaphorin) 5B)

Nucleotide

Genbank accession no. AB040878

Genbank version no. AB040878.1 GI:7959148

Genbank record update date: Aug. 2, 2006 05:40 PM

Polypeptide

Genbank accession no. BAA95969

Genbank version no. BAA95969.1 GI:7959149

Genbank record update date: Aug. 2, 2006 05:40 PM

### Cross References

Nagase T., et al (2000) DNA Res. 7 (2):143-150); WO2004/000997 (Claim 1); WO2003/003984 (Claim 1); WO2002/06339 (Claim 1; Page 50); WO2001/88133 (Claim 1; Page 41-43, 48-58); WO2003/054152 (Claim 20); WO2003/101400 (Claim 11); Accession: 30 Q9P283; Genew; HGNC:10737

(8) PSCA hIg (2700050C12Rik, C530008O16Rik, RIKEN cDNA 2700050C12, RIKEN cDNA 2700050C12 gene)

Nucleotide

Genbank accession no. AY358628

Genbank version no. AY358628.1 GI:37182377

Genbank record update date: Dec. 1, 2009 04:15 AM

Polypeptide

Genbank accession no. AAQ88991

Genbank version no. AAQ88991.1 GI:37182378

Genbank record update date: Dec. 1, 2009 04:15 AM

### Cross References

Ross et al (2002) Cancer Res. 62:2546-2553; US2003/129192 (Claim 2); US2004/044180 (Claim 12); US2004/044179 35 (Claim 11); US2003/096961 (Claim 11); US2003/232056 (Example 5); WO2003/105758 16 (Claim 12); US2003/206918 (Example 5); EP1347046 (Claim 1); WO2003/025148 (Claim 20); GI:37182378.

(9) ETBR (Endothelin Type B Receptor)

Nucleotide

Genbank accession no. AY275463

Genbank version no. AY275463.1 GI:30526094

Genbank record update date: Mar. 11, 2010 02:26 AM

Polypeptide

Genbank accession no. AAP32295

Genbank version no. AAP32295.1 GI:30526095

Genbank record update date: Mar. 11, 2010 02:26 AM

### Cross References

Nakamuta M., et al Biochem. Biophys. Res. Commun. 177, 34-39, 1991; Ogawa Y., et al Biochem. Biophys. Res. Commun. 178, 248-255, 1991; Arai H., et al Jpn. Circ. J. 56, 1303-1307, 1992; Arai H., et al J. Biol. Chem. 268, 3463-3470, 1993; Sakamoto A., Yanagisawa M., et al Biochem. Biophys. Res. Commun. 178, 656-663, 1991; Elshourbagy N. A., et al J. Biol. Chem. 268, 3873-3879, 1993; Haendler B., et al J. Cardiovasc. Pharmacol. 20, s1-S4, 1992; Tsutsumi M., et al Gene 228, 43-49, 1999; Strausberg R. L., et al Proc. Natl. Acad. Sci. U.S.A. 99, 16899-16903, 2002; Bourgeois C., et al J. Clin. Endocrinol. Metab. 82, 3116-3123, 1997; Okamoto Y., et al Biol. Chem. 272, 21589-21596, 1997; Verheij J. B., et al Am. J. Med. Genet. 108, 223-225, 2002; Hofstra R. M. W., et al Eur. J. Hum. Genet. 5, 180-185, 1997; Puffenberger E. G., et al Cell 79, 1257-1266, 1994; Attie T., et al, Hum. Mol. Genet. 4, 2407-15 2409, 1995; Auricchio A., et al Hum. Mol. Genet. 5:351-354, 1996; Amiel J., et al Hum. Mol. Genet. 5, 355-357, 1996; Hofstra R. M. W., et al Nat. Genet. 12, 445-447, 1996; Svensson P. J., et al Hum. Genet. 103, 145-148, 1998; Fuchs S., et al Mol. Med. 7, 115-124, 2001; Pingault V., et al (2002) Hum. Genet. 111, 198-206; WO2004/045516 (Claim 1); WO2004/048938 (Example 2); WO2004/040000 (Claim 151); WO2003/087768 (Claim 1); 20 WO2003/016475 (Claim 1); WO2003/016475 (Claim 1); WO2002/61087 (FIG. 1); WO2003/016494 (FIG. 6); WO2003/025138 (Claim 12; Page 144); WO2001/98351 (Claim 1; Page 124-125); EP0522868 (Claim 8; FIG. 2); WO2001/77172 (Claim 1; Page 297-299); US2003/109676; U.S. Pat. No. 6,518,404 (FIG. 3); U.S. Pat. No. 5,773,223 (Claim 1a; Col 31-34); WO2004/001004.

(10) MSG783 (RNF124, Hypothetical Protein FLJ20315)

Nucleotide

Genbank accession no. NM_017763

Genbank version no. NM_017763.4 GI:167830482

Genbank record update date: Jul. 22, 2012 12:34 AM

Polypeptide

Genbank accession no. NP_060233

Genbank version no. NP_060233.3 GI:56711322

Genbank record update date: Jul. 22, 2012 12:34 AM

### Cross References

WO2003/104275 (Claim 1); WO2004/046342 (Example 2); WO2003/042661 (Claim 12); WO2003/083074 (Claim 14; Page 61); WO2003/018621 (Claim 1); WO2003/024392 (Claim 2; FIG. 93); WO2001/66689 (Example 6); LocusID:54894.

(11) STEAP2 (HGNC_8639, IPCA-1, PCANAP1, STAMP1, STEAP2, STMP, Prostate Cancer Associated Gene 1, Prostate Cancer Associated Protein 1, Six Transmembrane Epithelial Antigen of Prostate 2, Six Transmembrane Prostate Protein)

Nucleotide

Genbank accession no. AF455138

Genbank version no. AF455138.1 GI:22655487

Genbank record update date: Mar. 11, 2010 01:54 AM

Polypeptide

Genbank accession no. AAN04080

Genbank version no. AAN04080.1 GI:22655488

Genbank record update date: Mar. 11, 2010 01:54 AM

### Cross References

Lab. Invest. 82 (11):1573-1582 (2002)); WO2003/087306; US2003/064397 (Claim 1; FIG. 1); WO2002/72596 (Claim 13; Page 54-55); WO2001/72962 (Claim 1; FIG. 4B); 35 WO2003/104270 (Claim 11); WO2003/104270 (Claim 16); US2004/005598 (Claim 22); WO2003/042661 (Claim 12); US2003/060612 (Claim 12; FIG. 10); WO2002/26822 (Claim 23; FIG. 2); WO2002/16429 (Claim 12; FIG. 10); GI:22655488.

(12) TrpM4 (BR22450, FLJ20041, TRPM4, TRPM4B, Transient Receptor Potential Cation 5 Channel, Subfamily M, Member 4)

Nucleotide

Genbank accession no. NM_017636

Genbank version no. NM_017636.3 GI:304766649

Genbank record update date: Jun. 29, 2012 11:27 AM

Polypeptide

Genbank accession no. NP_060106

Genbank version no. NP_060106.2 GI:21314671

Genbank record update date: Jun. 29, 2012 11:27 AM

### Cross References

Xu, X. Z., et al Proc. Natl. Acad. Sci. U.S.A. 98 (19):10692-10697 (2001), Cell 109 (3):397-407 (2002), J. Biol. Chem. 278 (33):30813-30820 (2003)); US2003/143557 (Claim 4); WO2000/40614 (Claim 14; Page 100-103); WO2002/10382 (Claim 1; FIG. 9A); WO2003/042661 (Claim 12); WO2002/30268 (Claim 27; Page 391); US2003/219806 (Claim 4); WO2001/62794 (Claim 10 14; FIG. 1A-D); MIM:606936.

(13) CRIPTO (CR, CR1, CRGF, CRIPTO, TDGF1, Teratocarcinoma-Derived Growth Factor)

Nucleotide

Genbank accession no. NM_003212

Genbank version no. NM_003212.3 GI:292494881

Genbank record update date: Sep. 23, 2012 02:27 PM

Polypeptide

Genbank accession no. NP_003203

Genbank version no. NP_003203.1 GI:4507425

Genbank record update date: Sep. 23, 2012 02:27 PM

### Cross References

Ciccodicola, A., et al EMBO J. 8 (7):1987-1991 (1989), Am. J. Hum. Genet. 49 (3):555-565 (1991)); US2003/224411 (Claim 1); WO2003/083041 (Example 1); WO2003/034984 (Claim 12); WO2002/88170 (Claim 2; Page 52-53); WO2003/024392 (Claim 2; FIG. 58); WO2002/16413 (Claim 1; Page 94-95, 105); WO2002/22808 (Claim 2; FIG. 1); U.S. Pat. No. 5,854,399 (Example 2; Col 17-18); U.S. Pat. No. 5,792,616 (FIG. 2); MIM:187395.

(14) CD21 (CR2 (Complement Receptor 2) or C3DR (C3d/Epstein Barr Virus Receptor) or Hs.73792)

Nucleotide

Genbank accession no M26004

Genbank version no. M26004.1 GI:181939

Genbank record update date: Jun. 23, 2010 08:47 AM

Polypeptide

Genbank accession no. AAA35786

Genbank version no. AAA35786.1 GI:181940

Genbank record update date: Jun. 23, 2010 08:47 AM

### Cross References

Fujisaku et al (1989) J. Biol. Chem. 264 (4):2118-2125); Weis J. J., et al J. Exp. Med. 167, 1047-1066, 1988; Moore M., et al Proc. Natl. Acad. Sci. U.S.A. 84, 9194-9198, 1987; Barel M., et al Mol. Immunol. 35, 1025-1031, 1998; Weis J. J., et al Proc. Natl. Acad. Sci. U.S.A. 83, 5639-5643, 1986; Sinha S. K., et al (1993) J. Immunol. 150, 5311-5320; WO2004/045520 (Example 4); US2004/005538 (Example 1); WO2003/062401 (Claim 9); WO2004/045520 (Example 4); WO91/02536 (FIG. 9.1-9.9); WO2004/020595 (Claim 1); Accession: P20023; Q13866; Q14212; EMBL; M26004; AAA35786.1.

(15) CD79b (CD79B, CD79β, IGb (Immunoglobulin-Associated Beta), B29)

Nucleotide

Genbank accession no NM_000626

Genbank version no. NM_000626.2 GI:90193589

Genbank record update date: Jun. 26, 2012 01:53 PM

Polypeptide

Genbank accession no. NP_000617

Genbank version no. NP_000617.1 GI:11038674

Genbank record update date: Jun. 26, 2012 01:53 PM

### Cross References

Proc. Natl. Acad. Sci. U.S.A. (2003) 100 (7):4126-4131, Blood (2002) 100 (9):3068-3076, Muller et al (1992) Eur. J. Immunol. 22 (6):1621-1625); WO2004/016225 (claim 2, FIG. 140); WO2003/087768, US2004/101874 (claim 1, page 102); WO2003/062401 (claim 9); WO2002/78524 (Example 2); US2002/150573 (claim 35 5, page 15); U.S. Pat. No. 5,644,033; WO2003/048202 (claim 1, pages 306 and 309); WO 99/58658, U.S. Pat. No. 6,534,482 (claim 13, FIG. 17A/B); WO2000/55351 (claim 11, pages 1145-1146); MIM:147245

(16) FcRH2 (IFGP4, IRTA4, SPAP1A (SH2 Domain Containing Phosphatase Anchor Protein 5 1a), SPAP1B, SPAP1C)

Nucleotide

Genbank accession no NM_030764

Genbank version no. NM_030764.3 GI:227430280

Genbank record update date: Jun. 30, 2012 12:30 AM

Polypeptide

Genbank accession no. NP_110391

Genbank version no. NP_110391.2 GI:19923629

Genbank record update date: Jun. 30, 2012 12:30 AM

### Cross References

AY358130); Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al (2001) Biochem. Biophys. Res. Commun. 280 (3):768-775; WO2004/016225 (Claim 2); WO2003/077836; WO2001/38490 (Claim 5; FIG. 18D-1-18D-2); WO2003/097803 (Claim 12);
10 WO2003/089624 (Claim 25); MIM:606509.

(17) HER2 (ErbB2)

Nucleotide

Genbank accession no M11730

Genbank version no. M11730.1 GI:183986

Genbank record update date: Jun. 23, 2010 08:47 AM

Polypeptide

Genbank accession no. AAA75493

Genbank version no. AAA75493.1 GI:306840

Genbank record update date: Jun. 23, 2010 08:47 AM

### Cross References

Coussens L., et al Science (1985) 230(4730):1132-1139); Yamamoto T., et al Nature 319, 230-234, 1986; Semba K., et al Proc. Natl. Acad. Sci. U.S.A. 82, 6497-6501, 1985; Swiercz J. M., et al J. Cell Biol. 165, 869-15 880, 2004; Kuhns J. J., et al J. Biol. Chem. 274, 36422-36427, 1999; Cho H.-S., et al Nature 421, 756-760, 2003; Ehsani A., et al (1993) Genomics 15, 426-429; WO2004/048938 (Example 2); WO2004/027049 (FIG. 1I); WO2004/009622; WO2003/081210;
WO2003/089904 (Claim 9); WO2003/016475 (Claim 1); US2003/118592; WO2003/008537 (Claim 1); WO2003/055439 (Claim 29; FIG. 1A-B); WO2003/025228 (Claim 37; FIG. 5C);
20 WO2002/22636 (Example 13; Page 95-107); WO2002/12341 (Claim 68; FIG. 7); WO2002/13847 (Page 71-74); WO2002/14503 (Page 114-117); WO2001/53463 (Claim 2; Page 41-46); WO2001/41787 (Page 15); WO2000/44899 (Claim 52; FIG. 7); WO2000/20579 (Claim 3; FIG. 2); U.S. Pat. No. 5,869,445 (Claim 3; Col 31-38); WO9630514 (Claim 2; Page 56-61); EP1439393 (Claim 7); WO2004/043361 (Claim 7); WO2004/022709; WO2001/00244
25 (Example 3; FIG. 4); Accession: P04626; EMBL; M11767; AAA35808.1. EMBL; M11761; AAA35808.1

Antibodies

Abbott: US20110177095
For example, an antibody comprising CDRs having overall at least 80% sequence identity to CDRs having amino acid sequences of SEQ ID NO:3 (CDR-H1), SEQ ID NO:4 (CDR-H2), SEQ ID NO:5 (CDR-H3), SEQ ID NO:104 and/orSEQ ID NO:6 (CDR-L1), SEQ ID NO:7 (CDR-L2), and SEQ ID NO:8 (CDR-L3), wherein the anti-HER2 antibody or anti-HER2 binding fragment has reduced immunogenicity as compared to an antibody having a VH of SEQ ID NO:1 and a VL of SEQ ID NO:2.

Biogen: US20100119511
For example, ATCC accession numbers: PTA-10355, PTA-10356, PTA-10357, PTA-10358
For example, a purified antibody molecule that binds to HER2 comprising a all six CDR's from an antibody selected from the group consisting of BIIB71F10 (SEQ ID NOs:11, 13), BIIB69A09 (SEQ ID NOs:15, 17); BIIB67F10 (SEQ ID NOs:19, 21); BIIB67F11 (SEQ ID NOs:23, 25), BIIB66A12 (SEQ ID NOs:27, 29), BIIB66C01 (SEQ ID NOs:31, 33), BIIB65C10 (SEQ ID NOs:35, 37), BIIB65H09 (SEQ ID NOs:39, 41) and BIIB65B03 (SEQ ID NOs:43, 45), or CDRs which are identical or which have no more than two alterations from said CDRs.

Herceptin (Genentech)—U.S. Pat. No. 6,054,297; ATCC accession no. CRL-10463 (Genentech)

Pertuzumab (Genentech)
US20110117097
   for example, see SEQ IDs No. 15&16, SEQ IDs No. 17&18, SEQ IDs No. 23&24 & ATCC accession numbers HB-12215, HB-12216, CRL 10463, HB-12697.
US20090285837
US20090202546
   for example, ATCC accession numbers: HB-12215, HB-12216, CRL 10463, HB-12698.
US20060088523
   for example, ATCC accession numbers: HB-12215, HB-12216
   for example, an antibody comprising the variable light and variable heavy amino acid sequences in SEQ ID Nos. 3 and 4, respectively.
   for example, an antibody comprising a light chain amino acid sequence selected from SEQ ID No. 15 and 23, and a heavy chain amino acid sequence selected from SEQ ID No. 16 and 24
US20060018899
   for example, ATCC accession numbers: (7C2) HB-12215, (7F3) HB-12216, (4D5) CRL-10463, (2C4) HB-12697.
   for example, an antibody comprising the amino acid sequence in SEQ ID No. 23, or a deamidated and/or oxidized variant thereof.
US2011/0159014
   for example, an antibody having a light chain variable domain comprising the hypervariable regions of SEQ ID NO: 1”.
   For example, an antibody having a heavy chain variable domain comprising the hypervariable regions of SEQ ID NO: 2.
US20090187007

Glycotope: TrasGEX antibody http://www.glycotope.com/pipeline
For example, see International Joint Cancer Institute and Changhai Hospital Cancer Cent: HMTI-Fc Ab—Gao J., et al BMB Rep. 2009 Oct. 31; 42(10):636-41.

Symphogen: US20110217305

Union Stem Cell &Gene Engineering, China—Liu H Q., et al Xi Bao Yu Fen Zi Mian Yi Xue Za Zhi. 2010 May; 26(5):456-8.

(18) NCA (CEACAM6)

Nucleotide

Genbank accession no M18728

Genbank version no. M18728.1 GI:189084

Genbank record update date: Jun. 23, 2010 08:48 AM

Polypeptide

Genbank accession no. AAA59907

Genbank version no. AAA59907.1 GI:189085

Genbank record update date: Jun. 23, 2010 08:48 AM

### Cross References

Barnett T., et al Genomics 3, 59-66, 1988; Tawaragi Y., et al Biochem. Biophys. Res. Commun. 150, 89-96, 1988; Strausberg R. L., et al Proc. Natl. Acad. Sci. U.S.A. 99:16899-16903, 2002; WO2004/063709; EP1439393 (Claim 7); WO2004/044178 (Example 4); WO2004/031238; WO2003/042661 (Claim 12); WO2002/78524 (Example 2); WO2002/86443 (Claim 27; Page 427); WO2002/60317 (Claim 2); Accession: P40199; Q14920; EMBL; M29541; AAA59915.1.
EMBL; M18728.

(19) MDP (DPEP1)

Nucleotide

Genbank accession no BC017023

Genbank version no. BC017023.1 GI:16877538

Genbank record update date: Mar. 6, 2012 01:00 PM

Polypeptide

Genbank accession no. AAH17023

Genbank version no. AAH17023.1 GI:16877539

Genbank record update date: Mar. 6, 2012 01:00 PM

### Cross References

Proc. Natl. Acad. Sci. U.S.A. 99 (26):16899-16903 (2002)); WO2003/016475 (Claim 1); WO2002/64798 (Claim 33; Page 85-87); JP05003790 (FIG. 6-8); WO99/46284 (FIG. 9); MIM:179780.

(20) IL20R-Alpha (IL20Ra, ZCYTOR7)

Nucleotide

Genbank accession no AF184971

Genbank version no. AF184971.1 GI:6013324

Genbank record update date: Mar. 10, 2010 10:00 PM

Polypeptide

Genbank accession no. AAF01320

Genbank version no. AAF01320.1 GI:6013325

Genbank record update date: Mar. 10, 2010 10:00 PM

### Cross References

Clark H. F., et al Genome Res. 13, 2265-2270, 2003; Mungall A. J., et al Nature 425, 805-811, 2003; Blumberg H., et al Cell 104, 9-19, 2001; Dumoutier L., et al J. Immunol. 167, 3545-3549, 2001; Parrish-Novak J., et al J. Biol. Chem. 277, 47517-47523, 2002; Pletnev S., et al (2003) 10 Biochemistry 42:12617-12624; Sheikh F., et al (2004) J. Immunol. 172, 2006-2010; EP1394274 (Example 11); US2004/005320 (Example 5); WO2003/029262 (Page 74-75); WO2003/002717 (Claim 2; Page 63); WO2002/22153 (Page 45-47); US2002/042366 (Page 20-21); WO2001/46261 (Page 57-59); WO2001/46232 (Page 63-65); WO98/37193 (Claim 1; Page 55-59); Accession: Q9UHF4; Q6UWA9; Q96SH8; EMBL; AF184971; AAF01320.1.

(21) Brevican (BCAN, BEHAB)

Nucleotide

Genbank accession no AF229053

Genbank version no. AF229053.1 GI:10798902

Genbank record update date: Mar. 11, 2010 12:58 AM

Polypeptide

Genbank accession no. AAG23135

Genbank version no. AAG23135.1 GI:10798903

Genbank record update date: Mar. 11, 2010 12:58 AM

### Cross References

Gary S. C., et al Gene 256, 139-147, 2000; Clark H. F., et al Genome Res. 13, 2265-2270, 2003; Strausberg R. L., et al Proc. Natl. Acad. Sci. U.S.A. 99, 16899-16903, 2002; US2003/186372 (Claim 11); US2003/186373 (Claim 11); US2003/119131 (Claim 1; FIG. 52); US2003/119122 (Claim 1; 20 FIG. 52); US2003/119126 (Claim 1); US2003/119121 (Claim 1; FIG. 52); US2003/119129 (Claim 1); US2003/119130 (Claim 1); US2003/119128 (Claim 1; FIG. 52); US2003/119125 (Claim 1); WO2003/016475 (Claim 1); WO2002/02634 (Claim 1)

(22) EphB2R (DRT, ERK, Hek5, EPHT3, Tyro5)

Nucleotide

Genbank accession no NM_004442

Genbank version no. NM_004442.6 GI:111118979

Genbank record update date: Sep. 8, 2012 04:43 PM

Polypeptide

Genbank accession no. NP_004433

Genbank version no. NP_004433.2 GI:21396504

Genbank record update date: Sep. 8, 2012 04:43 PM

### Cross References

Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196:177-244 (2000)); WO2003042661 (Claim 12); WO200053216 (Claim 1; Page 41); WO2004065576 (Claim 1); WO2004020583 (Claim 9); WO2003004529 (Page 128-132); WO200053216 (Claim 1; Page 42); MIM:600997.

(23) ASLG659 (B7h)

Nucleotide

Genbank accession no. AX092328

Genbank version no. AX092328.1 GI:13444478

Genbank record update date: Jan. 26, 2011 07:37 AM

### Cross References

US2004/0101899 (Claim 2); WO2003104399 (Claim 11); WO2004000221 (FIG. 3); US2003/165504 (Claim 1); US2003/124140 (Example 2); US2003/065143 (FIG. 60); WO2002/102235 (Claim 13; Page 299); US2003/091580 (Example 2); WO2002/10187 (Claim 6; FIG. 10); WO2001/94641 (Claim 12; FIG. 7b); WO2002/02624 (Claim 13; FIG. 1A-1B); US2002/034749 (Claim 54; Page 45-46); WO2002/06317 (Example 2; Page 320-321, Claim 34; Page 321-322); WO2002/71928 (Page 468-469); WO2002/02587 (Example 1; FIG. 1); WO2001/40269 (Example 3; Pages 190-192); WO2000/36107 (Example 2; Page 205-207); WO2004/053079 (Claim 12); WO2003/004989 (Claim 1); WO2002/71928 (Page 233-234, 452-453); WO 01/16318.

(24) PSCA (Prostate Stem Cell Antigen Precursor)

Nucleotide

Genbank accession no AJ297436

Genbank version no. AJ297436.1 GI:9367211

Genbank record update date: Feb. 1, 2011 11:25 AM

Polypeptide

Genbank accession no. CAB97347

Genbank version no. CAB97347.1 GI:9367212

Genbank record update date: Feb. 1, 2011 11:25 AM

### Cross References

Reiter R. E., et al Proc. Natl. Acad. Sci. U.S.A. 95, 1735-1740, 1998; Gu Z., et al Oncogene 19, 1288-1296, 2000; Biochem. Biophys. Res. Commun. (2000) 275(3):783-788; WO2004/022709; EP1394274 (Example 11); US2004/018553 (Claim 17); WO2003/008537 (Claim 1); WO2002/81646 (Claim 1; Page 164); WO2003/003906 (Claim 10; Page 288); WO2001/40309 (Example 1; FIG. 17); US2001/055751 (Example 1; FIG. 1b); WO2000/32752 (Claim 18; FIG. 1); WO98/51805 (Claim 17; Page 97); WO98/51824 (Claim 10; Page 94); WO98/40403 (Claim 2; FIG. 1B); Accession: O43653; EMBL; AF043498; AAC39607.1

(25) GEDA

Nucleotide

Genbank accession no AY260763

Genbank version no. AY260763.1 GI:30102448

Genbank record update date: Mar. 11, 2010 02:24 AM

Polypeptide

Genbank accession no. AAP14954

Genbank version no. AAP14954.1 GI:30102449

Genbank record update date: Mar. 11, 2010 02:24 AM

### Cross References

AP14954 lipoma HMGIC fusion-partnerlike protein/pid=AAP14954.1—Homo sapiens (human); WO2003/054152 (Claim 20); WO2003/000842 (Claim 1); WO2003/023013 (Example 3, Claim 20); US2003/194704 (Claim 45); GI:30102449;

(26) BAFF-R (B Cell-Activating Factor Receptor, BLyS Receptor 3, BR3)

Nucleotide

Genbank accession no AF116456

Genbank version no. AF116456.1 GI:4585274

Genbank record update date: Mar. 10, 2010 09:44 PM

Polypeptide

Genbank accession no. AAD25356

Genbank version no. AAD25356.1 GI:4585275

Genbank record update date: Mar. 10, 2010 09:44 PM

### Cross References

BAFF receptor/pid=NP_443177.1—Homo sapiens: Thompson, J. S., et al Science 293 (5537), 2108-2111 (2001); WO2004/058309; WO2004/011611; WO2003/045422 (Example; Page 32-33); WO2003/014294 (Claim 35; FIG. 6B); WO2003/035846 (Claim 70; Page 615-616); WO2002/94852 (Col 136-137); WO2002/38766 25 (Claim 3; Page 133); WO2002/24909 (Example 3; FIG. 3); MIM:606269; NP_443177.1; NM_052945_1; AF132600

(27) CD22 (B-Cell Receptor CD22-B Isoform, BL-CAM, Lyb-8, Lyb8, SIGLEC-2, FLJ22814)

Nucleotide

Genbank accession no AK026467

Genbank version no. AK026467.1 GI:10439337

Genbank record update date: Sep. 11, 2006 11:24 PM

Polypeptide

Genbank accession no. BAB15489

Genbank version no. BAB15489.1 GI:10439338

Genbank record update date: Sep. 11, 2006 11:24 PM

### Cross References

Wilson et al (1991) J. Exp. Med. 173:137-146; 30 WO2003/072036 (Claim 1; FIG. 1); IM:107266; NP_001762.1; NM_001771_1.

(27a) CD22 (CD22 Molecule)

Nucleotide

Genbank accession no X52785

Genbank version no. X52785.1 GI:29778

Genbank record update date: Feb. 2, 2011 10:09 AM

Polypeptide

Genbank accession no. CAA36988

Genbank version no. CAA36988.1 GI:29779

Genbank record update date: Feb. 2, 2011 10:09 AM

### Cross References

Stamenkovic I. et al., Nature 345 (6270), 74-77 (1990)??

Other Information

Official Symbol: CD22

Other Aliases: SIGLEC-2, SIGLEC2

Other Designations: B-cell receptor CD22; B-lymphocyte cell adhesion molecule; BL-CAM; CD22 antigen; T-cell surface antigen Leu-14; sialic acid binding Ig-like lectin 2; sialic acid-binding Ig-like lectin 2

Antibodies

G5/44 (Inotuzumab): DiJoseph J F., et al Cancer Immunol Immunother. 2005 January; 54(1):11-24.

Epratuzumab-Goldenberg D M., et al Expert Rev Anticancer Ther. 6(10): 1341-53, 2006.

(28) CD79a (CD79A, CD79Alpha), Immunoglobulin-Associated Alpha, a B Cell-Specific Protein that Covalently Interacts with Ig Beta (CD79B) and Forms a Complex on the Surface with Ig M 35 Molecules, Transduces a Signal Involved in B-Cell Differentiation), pl: 4.84, MW: 25028 TM: 2

[P] Gene Chromosome: 19q13.2).

Nucleotide

Genbank accession no NM_001783

Genbank version no. NM_001783.3 GI:90193587

Genbank record update date: Jun. 26, 2012 01:48 PM

Polypeptide

Genbank accession no. NP_001774

Genbank version no. NP_001774.1 GI:4502685

Genbank record update date: Jun. 26, 2012 01:48 PM

### Cross References

WO2003/088808, US2003/0228319; WO2003/062401 (claim 9); US2002/150573 (claim 4, pages 13-14); WO99/58658 (claim 13, FIG. 16); WO92/07574 (FIG. 1); U.S. Pat. No. 5,644,033; Ha et al (1992) J. Immunol. 148(5):1526-1531; Müller et al (1992) Eur. J. Immunol. 22:1621-1625; Hashimoto et al (1994) Immunogenetics 40(4):287-295; Preud'homme et al (1992) Clin. Exp. 5 Immunol. 90(1):141-146; Yu et al (1992) J. Immunol. 148(2) 633-637; Sakaguchi et al (1988) EMBO J. 7(11):3457-3464

(29) CXCR5 (Burkitt's Lymphoma Receptor 1, a G Protein-Coupled Receptor that is Activated by the CXCL13 Chemokine, Functions in Lymphocyte Migration and Humoral Defense, Plays a 10 Role in HIV-2 Infection and Perhaps Development of AIDS, Lymphoma, Myeloma, and Leukemia); 372 aa, pl: 8.54 MW: 41959 TM: 7 [P] Gene Chromosome: 11q23.3,

Nucleotide

Genbank accession no NM_001716

Genbank version no. NM_001716.4 GI:342307092

Genbank record update date: Sep. 30, 2012 01:49 PM

Polypeptide

Genbank accession no. NP_001707

Genbank version no. NP_001707.1 GI:4502415

Genbank record update date: Sep. 30, 2012 01:49 PM

### Cross References

WO2004/040000; WO2004/015426; US2003/105292 (Example 2); U.S. Pat. No. 6,555,339 (Example 2); WO2002/61087 (FIG. 1); WO2001/57188 (Claim 20, page 269); WO2001/72830 (pages 12-13); WO2000/22129 (Example 1, pages 152-153, 15 Example 2, pages 254-256); WO99/28468 (claim 1, page 38); U.S. Pat. No. 5,440,021 (Example 2, col 49-52); WO94/28931 (pages 56-58); WO92/17497 (claim 7, FIG. 5); Dobner et al (1992) Eur. J. Immunol. 22:2795-2799; Barella et al (1995) Biochem. J. 309:773-779

(30) HLA-DOB (Beta Subunit of MHC Class II Molecule (Ia Antigen) that Binds Peptides and 20 Presents Them to CD4+ T Lymphocytes); 273 aa, pl: 6.56, MW: 30820.TM: 1 [P] Gene Chromosome: 6p21.3)

Nucleotide

Genbank accession no NM_002120

Genbank version no. NM_002120.3 GI:118402587

Genbank record update date: Sep. 8, 2012 04:46 PM

Polypeptide

Genbank accession no. NP_002111

Genbank version no. NP_002111.1 GI:4504403

Genbank record update date: Sep. 8, 2012 04:46 PM

### Cross References

Tonnelle et al (1985) EMBO J. 4(11):2839-2847; Jonsson et al (1989) Immunogenetics 29(6):411-413; Beck et al (1992) J. Mol. Biol. 228:433-441; Strausberg et al (2002) Proc. Natl. Acad. Sci USA 99:16899-16903; Servenius et al (1987) J. Biol. Chem. 262:8759-8766; Beck et al (1996) J. Mol. Biol. 25 255:1-13; Naruse et al (2002) Tissue Antigens 59:512-519; WO99/58658 (claim 13, FIG. 15); U.S. Pat. No. 6,153,408 (Col 35-38); U.S. Pat. No. 5,976,551 (col 168-170); U.S. Pat. No. 6,011,146 (col 145-146); Kasahara et al (1989) Immunogenetics 30(1):66-68; Larhammar et al (1985) J. Biol. Chem. 260(26):14111-14119

(31) P2X5 (Purinergic Receptor P2X Ligand-Gated Ion Channel 5, an Ion Channel Gated by Extracellular ATP, May be Involved in Synaptic Transmission and Neurogenesis, Deficiency May Contribute to the Pathophysiology of Idiopathic Detrusor Instability); 422 aa), pl: 7.63, MW: 47206 TM: 1 [P] Gene Chromosome: 17p13.3).

Nucleotide

Genbank accession no NM_002561

Genbank version no. NM_002561.3 GI:325197202

Genbank record update date: Jun. 27, 2012 12:41 AM

Polypeptide

Genbank accession no. NP_002552

Genbank version no. NP_002552.2 GI:28416933

Genbank record update date: Jun. 27, 2012 12:41 AM

### Cross References

Le et al (1997) FEBS Lett. 418(1-2):195-199; WO2004/047749; WO2003/072035 (claim 10); Touchman et al (2000) Genome Res. 10:165-173; WO2002/22660 (claim 20); WO2003/093444 (claim 1); WO2003/087768 (claim 1); WO2003/029277 (page 82)

(32) CD72 (B-Cell Differentiation Antigen CD72, Lyb-2); 359 aa, pl: 8.66, MW: 40225, TM: 1 5 [P] Gene Chromosome: 9p13.3).

Nucleotide

Genbank accession no NM_001782

Genbank version no. NM_001782.2 GI:194018444

Genbank record update date: Jun. 26, 2012 01:43 PM

Polypeptide

Genbank accession no. NP_001773

Genbank version no. NP_001773.1 GI:4502683

Genbank record update date: Jun. 26, 2012 01:43 PM

### Cross References

WO2004042346 (claim 65); WO2003/026493 (pages 51-52, 57-58); WO2000/75655 (pages 105-106); Von Hoegen et al (1990) J. Immunol. 144(12):4870-4877; Strausberg et al (2002) Proc. Natl. Acad. Sci USA 99:16899-16903.

(33) LY64 (Lymphocyte Antigen 64 (RP105), Type I Membrane Protein of the Leucine Rich Repeat (LRR) Family, Regulates B-Cell Activation and Apoptosis, Loss of Function is Associated with Increased Disease Activity in Patients with Systemic Lupus Erythematosis); 661 aa, pl: 6.20, MW: 74147 TM: 1 [P] Gene Chromosome: 5q12).

Nucleotide

Genbank accession no NM_005582

Genbank version no. NM_005582.2 GI:167555126

Genbank record update date: Sep. 2, 2012 01:50 PM

Polypeptide

Genbank accession no. NP_005573

Genbank version no. NP_005573.2 GI:167555127

Genbank record update date: Sep. 2, 2012 01:50 PM

### Cross References

US2002/193567; WO97/07198 (claim 11, pages 39-42); Miura et al (1996) 15 Genomics 38(3):299-304; Miura et al (1998) Blood 92:2815-2822; WO2003/083047; WO97/44452 (claim 8, pages 57-61); WO2000/12130 (pages 24-26).

(34) FcRH1 (Fc Receptor-Like Protein 1, a Putative Receptor for the Immunoglobulin Fc Domain that Contains C2 Type Ig-Like and ITAM Domains, May have a Role in B-Lymphocyte 20 Differentiation); 429 aa, pl: 5.28, MW: 46925 TM: 1 [P] Gene Chromosome: 1q21-1q22)

Nucleotide

Genbank accession no NM_052938

Genbank version no. NM_052938.4 GI:226958543

Genbank record update date: Sep. 2, 2012 01:43 PM

Polypeptide

Genbank accession no. NP_443170

Genbank version no. NP_443170.1 GI:16418419

Genbank record update date: Sep. 2, 2012 01:43 PM

### Cross References

WO2003/077836; WO2001/38490 (claim 6, FIG. 18E-1-18-E-2); Davis et al (2001) Proc. Natl. Acad. Sci USA 98(17):9772-9777; WO2003/089624 (claim 8); EP1347046 (claim 1); WO2003/089624 (claim 7).

(35) IRTA2 (Immunoglobulin Superfamily Receptor Translocation Associated 2, a Putative Immunoreceptor with Possible Roles in B Cell Development and Lymphomagenesis; Deregulation of the Gene by Translocation Occurs in Some B Cell Malignancies); 977 aa, pl: 6.88, MW: 106468, TM: 1 [P] Gene Chromosome: 1q21)

Nucleotide

Genbank accession no AF343662

Genbank version no. AF343662.1 GI:13591709

Genbank record update date: Mar. 11, 2010 01:16 AM

Polypeptide

Genbank accession no. AAK31325

Genbank version no. AAK31325.1 GI:13591710

Genbank record update date: Mar. 11, 2010 01:16 AM

### Cross References

AF343663, AF343664, AF343665, AF369794, AF397453, AK090423, AK090475, AL834187, AY358085; Mouse: AK089756, AY158090, AY506558; NP_112571.1; WO2003/024392 (claim 2, FIG. 97); Nakayama et al (2000) Biochem. Biophys. Res. Commun. 277(1):124-127; WO2003/077836; WO2001/38490 (claim 3, FIG. 18B-1-18B-2).

(36) TENB2 (TMEFF2, Tomoregulin, TPEF, HPP1, TR, Putative Transmembrane 35 Proteoglycan, Related to the EGF/Heregulin Family of Growth Factors and Follistatin); 374 aa)

Nucleotide

Genbank accession no AF179274

Genbank version no. AF179274.2 GI:12280939

Genbank record update date: Mar. 11, 2010 01:05 AM

Polypeptide

Genbank accession no. AAD55776

Genbank version no. AAD55776.2 GI:12280940

Genbank record update date: Mar. 11, 2010 01:05 AM

### Cross References

NCBI Accession: AAD55776, AAF91397, AAG49451, NCBI RefSeq: NP_057276; NCBI Gene: 23671; OMIM: 605734; SwissProt Q9UIK5; AY358907, CAF85723, CQ782436; WO2004/074320; JP2004113151; WO2003/042661; WO2003/009814; EP1295944 (pages 69-70); WO2002/30268 (page 329); WO2001/90304; US2004/249130; US2004/022727; WO2004/063355; US2004/197325; US2003/232350; 5 US2004/005563; US2003/124579; Horie et al (2000) Genomics 67:146-152; Uchida et al (1999) Biochem. Biophys. Res. Commun. 266:593-602; Liang et al (2000) Cancer Res. 60:4907-12; Glynne-Jones et al (2001) Int J Cancer. October 15; 94(2):178-84.

(37) PSMA-FOLH1 (Folate Hydrolase (Prostate-Specific Membrane Antigen) 1)

Nucleotide

Genbank accession no M99487

Genbank version no. M99487.1 GI:190663

Genbank record update date: Jun. 23, 2010 08:48 AM

Polypeptide

Genbank accession no. AAA60209

Genbank version no. AAA60209.1 GI:190664

Genbank record update date: Jun. 23, 2010 08:48 AM

### Cross References

Israeli R. S., et al Cancer Res. 53 (2), 227-230 (1993)

Other Information

Official Symbol: FOLH1

Other Aliases: GIG27, FGCP, FOLH, GCP2, GCPII, NAALAD1, NAALAdase, PSM, PSMA, mGCP

Other Designations: N-acetylated alpha-linked acidic dipeptidase 1; N-acetylated-alpha-linked acidic dipeptidase I; NAALADase I; cell growth-inhibiting gene 27 protein; folylpoly-gamma-glutamate carboxypeptidase; glutamate carboxylase II; glutamate carboxypeptidase 2; glutamate carboxypeptidase II; membrane glutamate carboxypeptidase; prostate specific membrane antigen variant F; pteroylpoly-gamma-glutamate carboxypeptidase

Antibodies

U.S. Pat. No. 7,666,425:

Antibodies produces by Hybridomas having the following ATCC references: ATCC accession No. HB-12101, ATCC accession No. HB-12109, ATCC accession No. HB-12127 and ATCC accession No. HB-12126.

Proscan: a monoclonal antibody selected from the group consisting of 8H12, 3E11, 17G1, 29B4, 30C1 and 20F2 (U.S. Pat. No. 7,811,564; Moffett S., et al Hybridoma (Larchmt). 2007 December; 26(6):363-72).

Cytogen: monoclonal antibodies 7E11-C5 (ATCC accession No. HB 10494) and 9H10-A4 (ATCC accession No. H1B11430)—U.S. Pat. No. 5,763,202

GlycoMimetics: NUH2—ATCC accession No. HB 9762 (U.S. Pat. No. 7,135,301)

Human Genome Science: HPRAJ70—ATCC accession No. 97131 (U.S. Pat. No. 6,824,993); Amino acid sequence encoded by the cDNA clone (HPRAJ70) deposited as American Type Culture Collection (“ATCC”) Deposit No. 97131

Medarex: Anti-PSMA antibodies that lack fucosyl residues—U.S. Pat. No. 7,875,278

Mouse anti-PSMA antibodies include the 3F5.4G6, 3D7.1.1, 4E10-1.14, 3E11, 4D8, 3E6, 3C9, 2C7, 1G3, 3C4, 3C6, 4D4, 1G9, 5C8B9, 3G6, 4C8B9, and monoclonal antibodies. Hybridomas secreting 3F5.4G6, 3D7.1.1, 4E10-1.14, 3E11, 4D8, 3E6, 3C9, 2C7, 1G3, 3C4, 3C6, 4D4, 1G9, 5C8B9, 3G6 or 4C8B9 have been publicly deposited and are described in U.S. Pat. No. 6,159,508. Relevant hybridomas have been publicly deposited and are described in U.S. Pat. No. 6,107,090. Moreover, humanized anti-PSMA antibodies, including a humanized version of J591, are described in further detail in PCT Publication WO 02/098897.

Other mouse anti-human PSMA antibodies have been described in the art, such as mAb 107-1A4 (Wang, S. et al. (2001) Int. J. Cancer 92:871-876) and mAb 2C9 (Kato, K. et al. (2003) Int. J. Urol. 10:439-444).

Examples of human anti-PSMA monoclonal antibodies include the 4A3, 7F12, 8C12, 8A11, 16F9, 2A10, 2C6, 2F5 and 1C3 antibodies, isolated and structurally characterized as originally described in PCT Publications WO 01/09192 and WO 03/064606 and in U.S. Provisional Application Ser. No. 60/654,125, entitled “Human Monoclonal Antibodies to Prostate Specific Membrane Antigen (PSMA)”, filed on Feb. 18, 2005. The V.sub.H amino acid sequences of 4A3, 7F12, 8C12, 8A11, 16F9, 2A10, 2C6, 2F5 and 1C3 are shown in SEQ ID NOs: 1-9, respectively. The V.sub.L amino acid sequences of 4A3, 7F12, 8C12, 8A11, 16F9, 2A10, 2C6, 2F5 and 1C3 are shown in SEQ ID NOs: 10-18, respectively.

Other human anti-PSMA antibodies include the antibodies disclosed in PCT Publication WO 03/034903 and US Application No. 2004/0033229.

NW Biotherapeutics: A hybridoma cell line selected from the group consisting of 3F5.4G6 having ATCC accession number HB12060, 3D7-1.I. having ATCC accession number HB12309, 4E10-1.14 having ATCC accession number HB12310, 3E11 (ATCC HB12488), 4D8 (ATCC HB12487), 3E6 (ATCC HB12486), 3C9 (ATCC HB12484), 2C7 (ATCC HB12490), 1G3 (ATCC HB12489), 3C4 (ATCC HB12494), 3C6 (ATCC HB12491), 4D4 (ATCC HB12493), 1G9 (ATCC HB12495), 5C8B9 (ATCC HB12492) and 3G6 (ATCC HB12485)—see U.S. Pat. No. 6,150,508

PSMA Development Company/Progenics/Cytogen—Seattle Genetics: mAb 3.9, produced by the hybridoma deposited under ATCC Accession No. PTA-3258 or mAb 10.3, produced by the hybridoma deposited under ATCC Accession No. PTA-3347—U.S. Pat. No. 7,850,971

PSMA Development Company—Compositions of PSMA antibodies (US 20080286284, Table 1)
This application is a divisional of U.S. patent application Ser. No. 10/395,894, filed on Mar. 21, 2003 (U.S. Pat. No. 7,850,971)

University Hospital Freiburg, Germany—mAbs 3/A12, 3/E7, and 3/F11 (Wolf P., et al Prostate. 2010 Apr. 1; 70(5):562-9).

(38) SST (Somatostatin Receptor; Note that there are 5 Subtypes)

(38.1) SSTR2 (Somatostatin Receptor 2)

Nucleotide

Genbank accession no NM_001050

Genbank version no. NM_001050.2 GI:44890054

Genbank record update date: Aug. 19, 2012 01:37 PM

Polypeptide

Genbank accession no. NP_001041

Genbank version no. NP_001041.1 GI:4557859

Genbank record update date: Aug. 19, 2012 01:37 PM

### Cross References

Yamada Y., et al Proc. Natl. Acad. Sci. U.S.A. 89 (1), 251-255 (1992); Susini C., et al Ann Oncol. 2006 December; 17(12):1733-42

Other Information

Official Symbol: SSTR2

Other Designations: SRIF-1; SS2R; somatostatin receptor type 2

(38.2) SSTR5 (Somatostatin Receptor 5)

Nucleotide

Genbank accession no D16827

Genbank version no. D16827.1 GI:487683

Genbank record update date: Aug. 1, 2006 12:45 PM

Polypeptide

Genbank accession no. BAA04107

Genbank version no. BAA04107.1 GI:487684

Genbank record update date: Aug. 1, 2006 12:45 PM

### Cross References

Yamada, Y., et al Biochem. Biophys. Res. Commun. 195 (2), 844-852 (1993)

Other Information

Official Symbol: SSTR5

Other Aliases: SS-5-R

Other Designations: Somatostatin receptor subtype 5; somatostatin receptor type 5

(38.3) SSTR1

(38.4) SSTR3

(38.5) SSTR4

AvB6—Both Subunits (39+40)

(39) ITGAV (Integrin, Alpha V;

Nucleotide

Genbank accession no M14648 J02826 M18365

Genbank version no. M14648.1 GI:340306

Genbank record update date: Jun. 23, 2010 08:56 AM

Polypeptide

Genbank accession no. AAA36808

Genbank version no. AAA36808.1 GI:340307

Genbank record update date: Jun. 23, 2010 08:56 AM

### Cross References

Suzuki S., et al Proc. Natl. Acad. Sci. U.S.A. 83 (22), 8614-8618 (1986)

Other Information

Official Symbol: ITGAV

Other Aliases: CD51, MSK8, VNRA, VTNR

Other Designations: antigen identified by monoclonal antibody L230; integrin alpha-V; integrin alphaVbeta3; integrin, alpha V (vitronectin receptor, alpha polypeptide, antigen CD51); vitronectin receptor subunit alpha

(40) ITGB6 (Integrin, Beta 6)

Nucleotide

Genbank accession no NM_000888

Genbank version no. NM_000888.3 GI:9966771

Genbank record update date: Jun. 27, 2012 12:46 AM

Polypeptide

Genbank accession no. NP_000879

Genbank version no. NP_000879.2 GI:9625002

Genbank record update date: Jun. 27, 2012 12:46 AM

### Cross References

Sheppard D. J., et al Biol. Chem. 265 (20), 11502-11507 (1990)

Other Information

Official Symbol: ITGB6

Other Designations: integrin beta-6

Antibodies

Biogen: U.S. Pat. No. 7,943,742—Hybridoma clones 6.3G9 and 6.8G6 were deposited with the ATCC, accession numbers ATCC PTA-3649 and -3645, respectively.

Biogen: U.S. Pat. No. 7,465,449—In some embodiments, the antibody comprises the same heavy and light chain polypeptide sequences as an antibody produced by hybridoma 6.1A8, 6.3G9, 6.8G6, 6.2B1, 6.2B10, 6.2A1, 6.2E5, 7.1G10, 7.7G5, or 7.1C5.

Centocor (J&J): U.S. Pat. Nos. 7,550,142; 7,163,681
For example in U.S. Pat. No. 7,550,142—an antibody having human heavy chain and human light chain variable regions comprising the amino acid sequences shown in SEQ ID NO: 7 and SEQ ID NO: 8.

Seattle Genetics: 15H3 (Ryan M C., et al Cancer Res Apr. 15, 2012; 72(8 Supplement): 4630)

(41) CEACAM5 (Carcinoembryonic Antigen-Related Cell Adhesion Molecule 5)

Nucleotide

Genbank accession no M17303

Genbank version no. M17303.1 GI:178676

Genbank record update date: Jun. 23, 2010 08:47 AM

Polypeptide

Genbank accession no. AAB59513

Genbank version no. AAB59513.1 GI:178677

Genbank record update date: Jun. 23, 2010 08:47 AM

### Cross References

Beauchemin N., et al Mol. Cell. Biol. 7 (9), 3221-3230 (1987)

Other Information

Official Symbol: CEACAM5

Other Aliases: CD66e, CEA

Other Designations: meconium antigen 100

Antibodies

AstraZeneca-MedImmune: US 20100330103; US20080057063;
US20020142359
   for example an antibody having complementarity determining regions (CDRs) with the following sequences: heavy chain; CDR1—DNYMH, CDR2—WIDPENGDTE YAPKFRG, CDR3—LIYAGYLAMD Y; and light chain CDR1—SASSSVTYMH, CDR2—STSNLAS, CDR3—QQRSTYPLT.
   Hybridoma 806.077 deposited as European Collection of Cell Cultures (ECACC) deposit no. 96022936.

Research Corporation Technologies, Inc.: U.S. Pat. No. 5,047,507

Bayer Corporation: U.S. Pat. No. 6,013,772

BioAlliance: U.S. Pat. Nos. 7,982,017; 7,674,605
U.S. Pat. No. 7,674,605
   an antibody comprising the heavy chain variable region sequence from the amino acid sequence of SEQ ID NO: 1, and the light chain variable region sequence from the amino acid sequence of SEQ ID NO:2.
   an antibody comprising the heavy chain variable region sequence from the amino acid sequence of SEQ ID NO:5, and the light chain variable region sequence from the amino acid sequence of SEQ ID NO:6.

Celltech Therapeutics Limited: U.S. Pat. No. 5,877,293

The Dow Chemical Company: U.S. Pat. Nos. 5,472,693; 6,417,337; 6,333,405
U.S. Pat. No. 5,472,693—for example, ATCC No. CRL-11215
U.S. Pat. No. 6,417,337—for example, ATCC CRL-12208
U.S. Pat. No. 6,333,405—for example, ATCC CRL-12208

Immunomedics, Inc: U.S. Pat. Nos. 7,534,431; 7,230,084; 7,300,644; 6,730,300;
US20110189085
   an antibody having CDRs of the light chain variable region comprise: CDR1 comprises KASQDVGTSVA (SEQ ID NO: 20); CDR2 comprises WTSTRHT (SEQ ID NO: 21); and CDR3 comprises QQYSLYRS (SEQ ID NO: 22);
   and the CDRs of the heavy chain variable region of said anti-CEA antibody comprise: CDR1 comprises TYWMS (SEQ ID NO: 23); CDR2 comprises EIHPDSSTINYAPSLKD (SEQ ID NO: 24); and CDR3 comprises LYFGFPWFAY (SEQ ID NO: 25).
US20100221175; US20090092598; US20070202044; US20110064653; US20090185974; US20080069775.

(42) MET (Met Proto-Oncogene; Hepatocyte Growth Factor Receptor)

Nucleotide

Genbank accession no M35073

Genbank version no. M35073.1 GI:187553

Genbank record update date: Mar. 6, 2012 11:12 AM

Polypeptide

Genbank accession no. AAA59589

Genbank version no. AAA59589.1 GI:553531

Genbank record update date: Mar. 6, 2012 11:12 AM

### Cross References

Dean M., et al Nature 318 (6044), 385-388 (1985)

Other Information

Official Symbol: MET

Other Aliases: AUTS9, HGFR, RCCP2, c-Met

Other Designations: HGF receptor; HGF/SF receptor; SF receptor; hepatocyte growth factor receptor; met proto-oncogene tyrosine kinase; proto-oncogene c-Met; scatter factor receptor; tyrosine-protein kinase Met

Antibodies

Abgenix/Pfizer: US20100040629
for example, the antibody produced by hybridoma 13.3.2 having American Type Culture Collection (ATCC) accession number PTA-5026; the antibody produced by hybridoma 9.1.2 having ATCC accession number PTA-5027; the antibody produced by hybridoma 8.70.2 having ATCC accession number PTA-5028; or the antibody produced by hybridoma 6.90.3 having ATCC accession number PTA-5029.

Amgen/Pfizer: US20050054019
for example, an antibody comprising a heavy chain having the amino acid sequences set forth in SEQ ID NO: 2 where X2 is glutamate and X4 is serine and a light chain having the amino acid sequence set forth in SEQ ID NO: 4 where X8 is alanine, without the signal sequences; an antibody comprising a heavy chain having the amino acid sequences set forth in SEQ ID NO: 6 and a light chain having the amino acid sequence set forth in SEQ ID NO: 8, without the signal sequences; an antibody comprising a heavy chain having the amino acid sequences set forth in SEQ ID NO: 10 and a light chain having the amino acid sequence set forth in SEQ ID NO: 12, without the signal sequences; or an antibody comprising a heavy chain having the amino acid sequences set forth in SEQ ID NO: 14 and a light chain having the amino acid sequence set forth in SEQ ID NO: 16, without the signal sequences.

Agouron Pharmaceuticals (Now Pfizer): US20060035907

Eli Lilly: US20100129369

Genentech: U.S. Pat. No. 5,686,292; US20100028337; US20100016241; US20070129301; US20070098707; US20070092520, US20060270594; US20060134104; US20060035278; US20050233960; US20050037431
U.S. Pat. No. 5,686,292—for example, ATCC HB-11894 and ATCC HB-11895 US 20100016241—for example, ATCC HB-11894 (hybridoma 1A3.3.13) or HB-11895 (hybridoma 5D5.11.6)

National Defense Medical Center, Taiwan: Lu R M., et al Biomaterials. 2011 April; 32(12):3265-74.

Novartis: US20090175860
for example, an antibody comprising the sequences of CDR1, CDR2 and CDR3 of heavy chain 4687, wherein the sequences of CDR1, CDR2, and CDR3 of heavy chain 4687 are residues 26-35, 50-65, and 98-102, respectively, of SEQ ID NO: 58; and the sequences of CDR1, CDR2, and CDR3 of light chain 5097, wherein the sequences of CDR1, CDR2, and CDR3 of light chain 5097 are residues 24-39, 55-61, and 94-100 of SEQ ID NO: 37.

Pharmacia Corporation: US20040166544

Pierre Fabre: US20110239316, US20110097262, US20100115639

Sumsung: US 20110129481—for example a monoclonal antibody produced from a hybridoma cell having accession number KCLRF-BP-00219 or accession number of KCLRF-BP-00223.

Samsung: US 20110104176—for example an antibody produced by a hybridoma cell having Accession Number: KCLRF-BP-00220.

University of Turin Medical School: DN-30 Pacchiana G., et al J Biol Chem. 2010 Nov. 12; 285(46):36149-57

Van Andel Research Institute: Jiao Y., et al Mol Biotechnol. 2005 September; 31(1):41-54.

(43) MUC1 (Mucin 1, Cell Surface Associated)

Nucleotide

Genbank accession no J05581

Genbank version no. J05581.1 GI:188869

Genbank record update date: Jun. 23, 2010 08:48 AM

Polypeptide

Genbank accession no. AAA59876

Genbank version no. AAA59876.1 GI:188870

Genbank record update date: Jun. 23, 2010 08:48 AM

### Cross References

Gendler S. J., et al J. Biol. Chem. 265 (25), 15286-15293 (1990)

Other Information

Official Symbol: MUC1

Other Aliases: RP11-263K19.2, CD227, EMA, H23AG, KL-6, MAM6, MUC-1, MUC-1/SEC, MUC-1/X, MUC1/ZD, PEM, PEMT, PUM

Other Designations: DF3 antigen; H23 antigen; breast carcinoma-associated antigen DF3; carcinoma-associated mucin; episialin; krebs von den Lungen-6; mucin 1, transmembrane; mucin-1; peanut-reactive urinary mucin; polymorphic epithelial mucin; tumor associated epithelial mucin; tumor-associated epithelial membrane antigen; tumor-associated mucin

Antibodies

AltaRex—Quest Pharma Tech: U.S. Pat. No. 6,716,966—for example an Alt-1 antibody produced by the hybridoma ATCC No PTA-975.

AltaRex—Quest Pharma Tech: U.S. Pat. No. 7,147,850

CRT: 5E5—Sørensen A L., et al Glycobiology vol. 16 no. 2 pp. 96-107, 2006; HMFG2—Burchell J., et al Cancer Res., 47, 5476-5482 (1987)

Glycotope GT-MAB: GT-MAB 2.5-GEX (Website: http://www.glycotope.com/pipeline/pankomab-gex)

Immunogen: U.S. Pat. No. 7,202,346
for example, antibody MJ-170: hybridoma cell line MJ-170 ATCC accession no. PTA-5286 Monoclonal antibody MJ-171: hybridoma cell line MJ-171 ATCC accession no. PTA-5287; monoclonal antibody MJ-172: hybridoma cell line MJ-172 ATCC accession no. PTA-5288; or monoclonal antibody MJ-173: hybridoma cell line MJ-173 ATCC accession no. PTA-5302

Immunomedics: U.S. Pat. No. 6,653,104

Ramot Tel Aviv Uni: U.S. Pat. No. 7,897,351

Regents Uni. CA: U.S. Pat. No. 7,183,388; US20040005647; US20030077676.

Roche GlycArt: U.S. Pat. No. 8,021,856

Russian National Cancer Research Center: Imuteran—Ivanov P K., et al Biotechnol J. 2007 July; 2(7):863-70

Technische Univ Braunschweig: (IIB6, HT186-B7, HT186-D11, HT186-G2, HT200-3A-C1, HT220-M-D1, HT220-M-G8)—Thie H., et al PLoS One. 2011 Jan. 14; 6(1):e15921

(44) CA9 (Carbonic Anhydrase IX)

Nucleotide

Genbank accession no. X66839

Genbank version no. X66839.1 GI:1000701

Genbank record update date: Feb. 2, 2011 10:15 AM

Polypeptide

Genbank accession no. CAA47315

Genbank version no. CAA47315.1 GI:1000702

Genbank record update date: Feb. 2, 2011 10:15 AM

### Cross References

Pastorek J., et al Oncogene 9 (10), 2877-2888 (1994)

Other Information

Official Symbol: CA9

Other Aliases: CAIX, MN

Other Designations: CA-IX; P54/58N; RCC-associated antigen G250; RCC-associated protein G250; carbonate dehydratase IX; carbonic anhydrase 9; carbonic dehydratase; membrane antigen MN; pMW1; renal cell carcinoma-associated antigen G250

Antibodies

Abgenix/Amgen: US20040018198

Affibody: Anti-CAIX Affibody molecules
(http://www.affibody.com/en/Product-Portfolio/Pipeline/)

Bayer: U.S. Pat. No. 7,462,696

Bayer/Morphosys: 3ee9 mAb—Petrul H M., et al Mol Cancer Ther. 2012 February; 11(2):340-9

Harvard Medical School: Antibodies G10, G36, G37, G39, G45, G57, G106, G119, G6, G27, G40 and G125. Xu C., et al PLoS One. 2010 Mar. 10; 5(3):e9625

Institute of Virology, Slovak Academy of Sciences (Bayer)—U.S. Pat. No. 5,955,075
for example, M75—ATCC Accession No. HB 11128 or MN12—ATCC Accession No. HB 11647

Institute of Virology, Slovak Academy of Sciences: U.S. Pat. No. 7,816,493
for example the M75 monoclonal antibody that is secreted from the hybridoma VU-M75, which was deposited at the American Type Culture Collection under ATCC No. HB 11128; or the V/10 monoclonal antibody secreted from the hybridoma V/10-VU, which was deposited at the International Depository Authority of the Belgian Coordinated Collection of Microorganisms (BCCM) at the Laboratorium voor Moleculaire Bioloqie-Plasmidencollectie (LMBP) at the Universeit Gent in Gent, Belgium, under Accession No. LMBP 6009CB.

Institute of Virology, Slovak Academy of Sciences US20080177046; US20080176310; US20080176258; US20050031623

Novartis: US20090252738

Wilex: U.S. Pat. No. 7,691,375—for example the antibody produced by the hybridoma cell line DSM ASC 2526.

Wilex: US20110123537; Rencarex: Kennett R H., et al Curr Opin Mol Ther. 2003 February; 5(1):70-5

Xencor: US20090162382

(45) EGFRvIII (Epidermal Growth Factor Receptor (EGFR), Transcript Variant 3,

Nucleotide

Genbank accession no. NM_201283

Genbank version no. NM_201283.1 GI:41327733

Genbank record update date: Sep. 30, 2012 01:47 PM

Polypeptide

Genbank accession no. NP_958440

Genbank version no. NP_958440.1 GI:41327734

Genbank record update date: Sep. 30, 2012 01:47 PM

### Cross-References

Batra S K., et al Cell Growth Differ 1995; 6:1251-1259.

Antibodies:

U.S. Pat. Nos. 7,628,986 and 7,736,644 (Amgen)
For example, a heavy chain variable region amino acid sequence selected from the group consisting of SEQ ID NO: 142 and variants & a light chain variable region amino acid sequence selected from the group consisting of: SEQ ID NO: 144 and variants.

US20100111979 (Amgen)
For example, an antibody comprising a heavy chain amino acid sequence comprising:
CDR1 consisting of a sequence selected from the group consisting of the amino acid sequences for the CDR1 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17);
CDR2 consisting of a sequence selected from the group consisting of the amino acid sequences for the CDR2 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17); and
CDR3 consisting of a sequence selected from the group consisting of the amino acid sequences for the CDR3 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

US20090240038 (Amgen)
For example, an antibody having at least one of the heavy or light chain polypeptides comprises an amino acid sequence that is at least 90% identical to the amino acid sequence selected from the group consisting of: SEQ ID NO: 2, SEQ ID NO: 19, SEQ ID NO: 142, SEQ ID NO: 144, and any combination thereof.

US20090175887 (Amgen)
For example, an antibody having a heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

US20090156790 (Amgen)
For example, antibody having heavy chain polypeptide and a light chain polypeptide, wherein at least one of the heavy or light chain polypeptides comprises an amino acid sequence that is at least 90% identical to the amino acid sequence selected from the group consisting of: SEQ ID NO: 2, SEQ ID NO: 19, SEQ ID NO: 142, SEQ ID NO: 144, and any combination thereof.

US20090155282, US20050059087 and US20050053608 (Amgen)
For example, an antibody heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

MR1-1 (U.S. Pat. No. 7,129,332; Duke)
For example, a variant antibody having the sequence of SEQ ID NO. 18 with the substitutions S98P-T99Y in the CDR3 VH, and F92W in CDR3 VL.

L8A4, H10, Y10 (Wikstrand C J., et al Cancer Res. 1995 Jul. 15; 55(14):3140-8; Duke)

US20090311803 (Harvard University)
For example, SEQ ID NO:9 for antibody heavy chain variable region, and SEQ ID NO: 3 for light chain variable region amino acid sequences

US20070274991 (EMD72000, also known as matuzumab; Harvard University)
For example, SEQ ID NOs: 3 & 9 for light chain and heavy chain respectively

U.S. Pat. No. 6,129,915 (Schering)
For example, SEQ. ID NOs: 1, 2, 3, 4, 5 and 6.

mAb CH12—Wang H., et al FASEB J. 2012 January; 26(1):73-80 (Shanghai Cancer Institute).

RAbDMvIII—Gupta P., et al BMC Biotechnol. 2010 Oct. 7; 10:72 (Stanford University Medical Center).

mAb Ua30—Ohman L., et al Tumour Biol. 2002 March-April; 23(2):61-9 (Uppsala University).

Han D G., et al Nan Fang Yi Ke Da Xue Xue Bao. 2010 January; 30(1):25-9 (Xi'an Jiaotong University).

(46) CD33 (CD33 Molecule)

Nucleotide

Genbank accession no. M_23197

Genbank version no. NM_23197.1 GI:180097

Genbank record update date: Jun. 23, 2010 08:47 AM

Polypeptide

Genbank accession no. AAA51948

Genbank version no. AAA51948.1 GI:188098

Genbank record update date: Jun. 23, 2010 08:47 AM

### Cross-References

Simmons D., et al J. Immunol. 141 (8), 2797-2800 (1988)

Other Information

Official Symbol: CD33

Other Aliases: SIGLEC-3, SIGLEC3, p 67

Other Designations: CD33 antigen (gp67); gp67; myeloid cell surface antigen CD33; sialic acid binding Ig-like lectin 3; sialic acid-binding Ig-like lectin

Antibodies

H195 (Lintuzumab)—Raza A., et al Leuk Lymphoma. 2009 August; 50(8):1336-44; U.S. Pat. No. 6,759,045 (Seattle Genetics/Immunomedics)

mAb OKT9: Sutherland, D. R. et al. Proc Natl Acad Sci USA 78(7): 4515-4519 1981, Schneider, C., et al J Biol Chem 257, 8516-8522 (1982)

mAb E6: Hoogenboom, H. R., et al J Immunol 144, 3211-3217 (1990)

U.S. Pat. No. 6,590,088 (Human Genome Sciences)
For example, SEQ ID NOs: 1 and 2 and ATCC accession no. 97521

U.S. Pat. No. 7,557,189 (Immunogen)
For example, an antibody or fragment thereof comprising a heavy chain variable region which comprises three CDRs having the amino acid sequences of SEQ ID NOs:1-3 and a light chain variable region comprising three CDRs having the amino acid sequences of SEQ ID NOs:4-6.

(47) CD19 (CD19 Molecule)

Nucleotide

Genbank accession no. NM_001178098

Genbank version no. NM_001178098.1 GI:296010920

Genbank record update date: Sep. 10, 2012 12:43 AM

Polypeptide

Genbank accession no. NP_001171569

Genbank version no. NP_001171569.1 GI:296010921

Genbank record update date: Sep. 10, 2012 12:43 AM

### Cross-References

Tedder T F., et al J. Immunol. 143 (2): 712-7 (1989)

Other Information

Official Symbol: CD19

Other Aliases: B4, CVID3

Other Designations: B-lymphocyte antigen CD19; B-lymphocyte surface antigen B4; T-cell surface antigen Leu-12; differentiation antigen CD19

Antibodies

Immunogen: HuB4—Al-Katib A M., et al Clin Cancer Res. 2009 Jun. 15; 15(12):4038-45.

4G7: Kügler M., et al Protein Eng Des Sel. 2009 March; 22(3):135-47
For example, sequences in FIG. 3 of of Knappik, A. et al. J Mol Biol 2000 February; 296(1):57-86

AstraZeneca/MedImmune: MEDI-551—Herbst R., et al J Pharmacol Exp Ther. 2010 October; 335(1):213-22

Glenmark Pharmaceuticals: GBR-401—Hou S., et al Mol Cancer Ther November 2011 10 (Meeting Abstract Supplement) C164

U.S. Pat. No. 7,109,304 (Immunomedics)
For example, an antibody comprising the sequence of hA19Vk (SEQ ID NO:7) and the sequence of hA19VH (SEQ ID NO:10)

U.S. Pat. No. 7,902,338 (Immunomedics)
For example, an antibody or antigen-binding fragment thereof that comprises the light chain complementarity determining region CDR sequences CDR1 of SEQ ID NO: 16 (KASQSVDYDGDSYLN); CDR2 of SEQ ID NO: 17 (DASNLVS); and CDR3 of SEQ ID NO: 18 (QQSTEDPWT) and the heavy chain CDR sequences CDR1 of SEQ ID NO: 19 (SYWMN); CDR2 of SEQ ID NO: 20 (QIWPGDGDTNYNGKFKG) and CDR3 of SEQ ID NO: 21 (RETTTVGRYYYAMDY) and also comprises human antibody framework (FR) and constant region sequences with one or more framework region amino acid residues substituted from the corresponding framework region sequences of the parent murine antibody, and wherein said substituted FR residues comprise the substitution of serine for phenylalanine at Kabat residue 91 of the heavy chain variable region.

Medarex: MDX-1342—Cardarelli P M., et al Cancer Immunol Immunother. 2010 February; 59(2):257-65.

MorphoSys/Xencor: MOR-208/XmAb-5574—Zalevsky J., et al Blood. 2009 Apr. 16; 113(16):3735-43

U.S. Pat. No. 7,968,687 (Seattle Genetics)
An antibody or antigen-binding fragment comprising a heavy chain variable domain comprising the amino acid sequence of SEQ ID NO:9 and a light chain variable domain comprising the amino acid sequence of SEQ ID NO: 24.

4G7 chim—Lang P., et al Blood. 2004 May 15; 103(10):3982-5 (University of Tübingen)
For example, FIG. 6 and SEQ ID No: 80 of US20120082664

Zhejiang University School of Medicine: 2E8—Zhang J., et al J Drug Target. 2010 November; 18(9):675-8

(48) IL2RA (Interleukin 2 Receptor, Alpha); NCBI Reference Sequence: NM_000417.2);

Nucleotide

Genbank accession no. NM_000417

Genbank version no. NM_000417.2 GI:269973860

Genbank record update date: Sep. 9, 2012 04:59 PM

Polypeptide

Genbank accession no. NP_000408

Genbank version no. NP_000408.1 GI:4557667

Genbank record update date: Sep. 9, 2012 04:59 PM

### Cross-References

Kuziel W. A., et al J. Invest. Dermatol. 94 (6 SUPPL), 27S-32S (1990)

Other Information

Official Symbol: IL2RA

Other Aliases: RP11-536K7.1, CD25, IDDM10, IL2R, TCGFR

Other Designations: FIL-2 receptor subunit alpha; IL-2-RA; IL-2R subunit alpha; IL2-RA; TAC antigen; interleukin-2 receptor subunit alpha; p55

Antibodies

U.S. Pat. No. 6,383,487 (Novartis/UCL: Baxilisimab [Simulect])

U.S. Pat. No. 6,521,230 (Novartis/UCL: Baxilisimab [Simulect])
For example, an antibody having an antigen binding site comprises at least one domain which comprises CDR1 having the amino acid sequence in SEQ. ID. NO: 7, CDR2 having the amino acid sequence in SEQ. ID. NO: 8, and CDR3 having the amino acid sequence in SEQ. ID. NO: 9; or said CDR1, CDR2 and CDR3 taken in sequence as a whole comprise an amino acid sequence which is at least 90% identical to SEQ. ID. NOs: 7, 8 and 9 taken in sequence as a whole.

Daclizumab—Rech A J., et al Ann N Y Acad Sci. 2009 September; 1174:99-106 (Roche)

(49) AXL (AXL Receptor Tyrosine Kinase)

Nucleotide

Genbank accession no. M76125

Genbank version no. M76125.1 GI:292869

Genbank record update date: Jun. 23, 2010 08:53 AM

Polypeptide

Genbank accession no. AAA61243

Genbank version no. AAA61243.1 GI:29870

Genbank record update date: Jun. 23, 2010 08:53 AM

### Cross-References

O'Bryan J. P., et al Mol. Cell. Biol. 11 (10), 5016-5031 (1991); Bergsagel P. L., et al J. Immunol. 148 (2), 590-596 (1992)

Other Information

Official Symbol: AXL

Other Aliases: JTK11, UFO

Other Designations: AXL oncogene; AXL transforming sequence/gene; oncogene AXL; tyrosine-protein kinase receptor UFO

Antibodies

YW327.6S2—Ye X., et al Oncogene. 2010 Sep. 23; 29(38):5254-64. (Genentech)

BergenBio: BGB324 (http://www.bergenbio.com/BGB324)

(50) CD30-TNFRSF8 (Tumor Necrosis Factor Receptor Superfamily, Member 8)

Nucleotide

Genbank accession no. M83554

Genbank version no. M83554.1 GI:180095

Genbank record update date: Jun. 23, 2010 08:53 AM

Polypeptide

Genbank accession no. AAA51947

Genbank version no. AAA51947.1 GI:180096

Genbank record update date: Jun. 23, 2010 08:53 AM

### Cross-References

Durkop H., et al Cell 68 (3), 421-427 (1992)

Other Information

Official Symbol: TNFRSF8

Other Aliases: CD30, D1S166E, Ki-1

Other Designations: CD30L receptor; Ki-1 antigen; cytokine receptor CD30; lymphocyte activation antigen CD30; tumor necrosis factor receptor superfamily member 8

(51) BCMA (B-Cell Maturation Antigen)-TNFRSF17 (Tumor Necrosis Factor Receptor Superfamily, Member 17)

Nucleotide

Genbank accession no. Z29574

Genbank version no. Z29574.1 GI:471244

Genbank record update date: Feb. 2, 2011 10:40 AM

Polypeptide

Genbank accession no. CAA82690

Genbank version no. CAA82690.1 GI:471245

Genbank record update date: Feb. 2, 2011 10:40 AM

### Cross-References

Laabi Y., et al Nucleic Acids Res. 22 (7), 1147-1154 (1994)

Other Information

Official Symbol: TNFRSF17

Other Aliases: BCM, BCMA, CD269

Other Designations: B cell maturation antigen; B-cell maturation factor; B-cell maturation protein; tumor necrosis factor receptor superfamily member 17

(52) CT Ags-CTA (Cancer Testis Antigens)

### Cross-References

Fratta E., et al. Mol Oncol. 2011 April; 5(2):164-82; Lim S H., at al Am J Blood Res. 2012; 2(1):29-35.

(53) CD174 (Lewis Y)-FUT3 (Fucosyltransferase 3 (Galactoside 3(4)-L-Fucosyltransferase, Lewis Blood Group)

Nucleotide

Genbank accession no. NM000149

Genbank version no. NM000149.3 GI:148277008

Genbank record update date: Jun. 26, 2012 04:49 PM

Polypeptide

Genbank accession no. NP_000140

Genbank version no. NP_000140.1 GI:4503809

Genbank record update date: Jun. 26, 2012 04:49 PM

### Cross-References

Kukowska-Latallo, J. F., et al Genes Dev. 4 (8), 1288-1303 (1990)

Other Information

Official Symbol: FUT3

Other Aliases: CD174, FT3B, FucT-III, LE, Les

Other Designations: Lewis FT; alpha-(1,3/1,4)-fucosyltransferase; blood group Lewis alpha-4-fucosyltransferase; fucosyltransferase III; galactoside 3(4)-L-fucosyltransferase

(54) CLEC14A (C-Type Lectin Domain Family 14, Member A; Genbank Accession No. NM175060)

Nucleotide

Genbank accession no. NM175060

Genbank version no. NM175060.2 GI:371123930

Genbank record update date: Apr. 1, 2012 03:34 PM

Polypeptide

Genbank accession no. NP_778230

Genbank version no. NP_778230.1 GI:28269707

Genbank record update date: Apr. 1, 2012 03:34 PM

Other Information

Official Symbol: CLEC14A

Other Aliases: UNQ236/PRO269, C14orf27, CEG1, EGFR-5

Other Designations: C-type lectin domain family 14 member A; CIECT and EGF-like domain containing protein; epidermal growth factor receptor 5

(55) GRP78-HSPA5 (Heat Shock 70 kDa Protein 5 (Glucose-Regulated Protein, 78 kDa)

Nucleotide

Genbank accession no. NM005347

Genbank version no. NM005347.4 GI:305855105

Genbank record update date: Sep. 30, 2012 01:42 PM

Polypeptide

Genbank accession no. NP_005338

Genbank version no. NP_005338.1 GI:16507237

Genbank record update date: Sep. 30, 2012 01:42 PM

### Cross-References

Ting J., et al DNA 7 (4), 275-286 (1988)

Other Information

Official Symbol: HSPA5

Other Aliases: BIP, GRP78, MIF2

Other Designations: 78 kDa glucose-regulated protein; endoplasmic reticulum lumenal Ca(2+)-binding protein grp78; immunoglobulin heavy chain-binding protein

(56) CD70 (CD70 Molecule) L08096

Nucleotide

Genbank accession no. L08096

Genbank version no. L08096.1 GI:307127

Genbank record update date: Jun. 23, 2010 08:54 AM

Polypeptide

Genbank accession no. AAA36175

Genbank version no. AAA36175.1 GI:307128

Genbank record update date: Jun. 23, 2010 08:54 AM

### Cross-References

Goodwin R. G., et al Cell 73 (3), 447-456 (1993)

Other Information

Official Symbol: CD70

Other Aliases: CD27L, CD27LG, TNFSF7

Other Designations: CD27 ligand; CD27-L; CD70 antigen; Ki-24 antigen; surface antigen CD70; tumor necrosis factor (ligand) superfamily, member 7; tumor necrosis factor ligand superfamily member 7

Antibodies

MDX-1411 against CD70 (Medarex)

h1F6 (Oflazoglu, E., et al, Clin Cancer Res. 2008 Oct. 1; 14(19):6171-80; Seattle Genetics)
For example, see US20060083736 SEQ ID NOs: 1, 2, 11 and 12 and FIG. 1.

(57) Stem Cell Specific Antigens. For Example:
5T4 (see entry (63) below)
CD25 (see entry (48) above)
CD32
   Polypeptide
      Genbank accession no. ABK42161
      Genbank version no. ABK42161.1 GI:117616286
      Genbank record update date: Jul. 25, 2007 03:00 PM
LGR5/GPR49
   Nucleotide
      Genbank accession no. NM_003667
      Genbank version no. NM_003667.2 GI:24475886
      Genbank record update date: Jul. 22, 2012 03:38 PM
   Polypeptide
      Genbank accession no. NP_003658
      Genbank version no. NP_003658.1 GI:4504379
      Genbank record update date: Jul. 22, 2012 03:38 PM
Prominin/CD133
   Nucleotide
      Genbank accession no. NM_006017
      Genbank version no. NM_006017.2 GI:224994187
      Genbank record update date: Sep. 30, 2012 01:47 PM
   Polypeptide
      Genbank accession no. NP_006008
      Genbank version no. NP_006008.1 GI:5174387
      Genbank record update date: Sep. 30, 2012 01:47 PM

(58) ASG-5

### Cross-References

(Smith L. M., et. al AACR 2010 Annual Meeting (abstract #2590); Gudas J. M., et. al. AACR 2010 Annual Meeting (abstract #4393)

Antibodies

Anti-AGS-5 Antibody: M6.131 (Smith, L. M., et. al AACR 2010 Annual Meeting (abstract #2590)

(59) ENPP3 (Ectonucleotide Pyrophosphatase/Phosphodiesterase 3)

Nucleotide

Genbank accession no. AF005632

Genbank version no. AF005632.2 GI:4432589

Genbank record update date: Mar. 10, 2010 09:41 PM

Polypeptide

Genbank accession no. AAC51813

Genbank version no. AAC51813.1 GI:2465540

Genbank record update date: Mar. 10, 2010 09:41 PM

### Cross-References

Jin-Hua P., et al Genomics 45 (2), 412-415 (1997)

Other Information

Official Symbol: ENPP3

Other Aliases: RP5-988G15.3, B10, CD203c, NPP3, PD-IBETA, PDNP3

Other Designations: E-NPP 3; dJ1005H11.3 (phosphodiesterase 1/nucleotide pyrophosphatase 3); dJ914N13.3 (phosphodiesterase I/nucleotide pyrophosphatase 3); ectonucleotide pyrophosphatase/phosphodiesterase family member 3; gp130RB13-6; phosphodiesterase I beta; phosphodiesterase I/nucleotide pyrophosphatase 3; phosphodiesterase-I beta

(60) PRR4 (Proline Rich 4 (Lacrimal))

Nucleotide

Genbank accession no. NM_007244

Genbank version no. NM_007244.2 GI:154448885

Genbank record update date: Jun. 28, 2012 12:39 PM

Polypeptide

Genbank accession no. NP_009175

Genbank version no. NP_009175.2 GI:154448886

Genbank record update date: Jun. 28, 2012 12:39 PM

### Cross-References

Dickinson D. P., et al Invest. Ophthalmol. Vis. Sci. 36 (10), 2020-2031 (1995)

Other Information

Official Symbol: PRR4

Other Aliases: LPRP, PROL4

Other Designations: lacrimal proline-rich protein; nasopharyngeal carcinoma-associated proline-rich protein 4; proline-rich polypeptide 4; proline-rich protein 4

(61) GCC-GUCY2C (Guanylate Cyclase 2C (Heat Stable Enterotoxin Receptor)

Nucleotide

Genbank accession no. NM_004963

Genbank version no. NM_004963.3 GI:222080082

Genbank record update date: Sep. 2, 2012 01:50 PM

Polypeptide

Genbank accession no. NP_004954

Genbank version no. NP_004954.2 GI:222080083

Genbank record update date: Sep. 2, 2012 01:50 PM

### Cross-References

De Sauvage F. J., et al J. Biol. Chem. 266 (27), 17912-17918 (1991); Singh S., et al Biochem. Biophys. Res. Commun. 179 (3), 1455-1463 (1991)

Other Information

Official Symbol: GUCY2C

Other Aliases: DIAR6, GUC2C, MUCIL, STAR

Other Designations: GC-C; STA receptor; guanylyl cyclase C; hSTAR; heat-stable enterotoxin receptor; intestinal guanylate cyclase

(62) Liv-1-SLC39A6 (Solute Carrier Family 39 (Zinc Transporter), Member 6)

Nucleotide

Genbank accession no. U41060

Genbank version no. U41060.2 GI:12711792

Genbank record update date: Nov. 30, 2009 04:35 PM

Polypeptide

Genbank accession no. AAA96258

Genbank version no. AAA96258.2 GI:12711793

Genbank record update date: Nov. 30, 2009 04:35 PM

### Cross-References

Taylor K M., et al Biochim Biophys Acta. 2003 Apr. 1; 1611(1-2):16-30

Other Information

Official Symbol: SLC39A6

Other Aliases: LIV-1

Other Designations: LIV-1 protein, estrogen regulated; ZIP-6; estrogen-regulated protein LIV-1; solute carrier family 39 (metal ion transporter), member 6; solute carrier family 39 member 6; zinc transporter ZIP6; zrt- and lrt-like protein 6

(63) 5T4, Trophoblast Glycoprotein, TPBG-TPBG (Trophoblast Glycoprotein)

Nucleotide

Genbank accession no. AJ012159

Genbank version no. AJ012159.1 GI:3805946

Genbank record update date: Feb. 1, 2011 10:27 AM

Polypeptide

Genbank accession no. CAA09930

Genbank version no. CAA09930.1 GI:3805947

Genbank record update date: Feb. 1, 2011 10:27 AM

### Cross-References

King K. W., et al Biochim. Biophys. Acta 1445 (3), 257-270 (1999)

Other Information
Official Symbol: TPBG
Other Aliases: 5T4, 5T4AG, M6P1
Other Designations: 5T4 oncofetal antigen; 5T4 oncofetal trophoblast glycoprotein; 5T4 oncotrophoblast glycoprotein

(64) CD56-NCMA1 (Neural Cell Adhesion Molecule 1)

Nucleotide

Genbank accession no. NM_000615

Genbank version no. NM_000615.6 GI:336285433

Genbank record update date: Sep. 23, 2012 02:32 PM

Polypeptide

Genbank accession no. NP_000606

Genbank version no. NP_000606.3 GI:94420689

Genbank record update date: Sep. 23, 2012 02:32 PM

### Cross-References

Dickson, G., et al, Cell 50 (7), 1119-1130 (1987)

Other Information

Official Symbol: NCAM1

Other Aliases: CD56, MSK39, NCAM

Other Designations: antigen recognized by monoclonal antibody 5.1H11; neural cell adhesion molecule, NCAM

Antibodies

Immunogen: HuN901 (Smith S V., et al Curr Opin Mol Ther. 2005 August; 7(4):394-401)
For example, see humanized from murine N901 antibody. See FIGS. 1b and 1e of Roguska, M. A., et al. Proc Natl Acad Sci USA February 1994; 91:969-973.

(65) CanAg (Tumor Associated Antigen CA242)

### Cross-References

Haglund C., et al Br J Cancer 60:845-851, 1989; Baeckstrom D., et al J Biol Chem 266:21537-21547, 1991

Antibodies

huC242 (Tolcher A W et al., J Clin Oncol. 2003 Jan. 15; 21(2):211-22; Immunogen)
For example, see US20080138898A1 SEQ ID NO: 1 and 2

(66) FOLR1 (Folate Receptor 1)

Nucleotide

Genbank accession no. J05013

Genbank version no. J05013.1 GI:182417

Genbank record update date: Jun. 23, 2010 08:47 AM

Polypeptide

Genbank accession no. AAA35823

Genbank version no. AAA35823.1 GI:182418

Genbank record update date: Jun. 23, 2010 08:47 AM

### Cross-References

Elwood P. C., et al J. Biol. Chem. 264 (25), 14893-14901 (1989)

Other Information

Official Symbol: FOLR1

Other Aliases: FBP, FOLR

Other Designations: FR-alpha; KB cells FBP; adult folate-binding protein; folate binding protein; folate receptor alpha; folate receptor, adult; ovarian tumor-associated antigen MOv18

Antibodies

M9346A—Whiteman K R., et al Cancer Res Apr. 15, 2012; 72(8 Supplement): 4628 (Immunogen)

(67) GPNMB (Glycoprotein (Transmembrane) nmb)

Nucleotide

Genbank accession no. X76534

Genbank version no. X76534.1 GI:666042

Genbank record update date: Feb. 2, 2011 10:10 AM

Polypeptide

Genbank accession no. CAA54044

Genbank version no. CAA54044.1 GI:666043

Genbank record update date: Feb. 2, 2011 10:10 AM

### Cross-References

Weterman M. A., et al Int. J. Cancer 60 (1), 73-81 (1995)

Other Information

Official Symbol: GPNMB

Other Aliases: UNQ1725/PRO9925, HGFIN, NMB

Other Designations: glycoprotein NMB; glycoprotein nmb-like protein; osteoactivin; transmembrane glycoprotein HGFIN; transmembrane glycoprotein NMB

Antibodies

Celldex Therapeutics: CR011 (Tse K F., et al Clin Cancer Res. 2006 Feb. 15; 12(4):1373-82)
For example, see EP1827492B1 SEQ ID NO: 22, 24, 26, 31, 33 and 35

(68) TIM-1-HAVCR1 (Hepatitis A Virus Cellular Receptor 1)

Nucleotide

Genbank accession no. AF043724

Genbank version no. AF043724.1 GI:2827453

Genbank record update date: Mar. 10, 2010 06:24 PM

Polypeptide

Genbank accession no. AAC39862

Genbank version no. AAC39862.1 GI:2827454

Genbank record update date: Mar. 10, 2010 06:24 PM

### Cross-References

Feigelstock D., et al J. Virol. 72 (8), 6621-6628 (1998)

Other Information

Official Symbol: HAVCR1

Other Aliases: HAVCR, HAVCR-1, KIM-1, KIM1, TIM, TIM-1, TIM1, TIMD-1, TIMD1

Other Designations: T cell immunoglobin domain and mucin domain protein 1; T-cell membrane protein 1; kidney injury molecule 1

(69) RG-1/Prostate Tumor Target Mindin—Mindin/RG-1

### Cross-References

Parry R., et al Cancer Res. 2005 Sep. 15; 65(18):8397-405

(70) B7-H4-VTCN1 (V-Set Domain Containing T Cell Activation Inhibitor 1

Nucleotide

Genbank accession no. BX648021

Genbank version no. BX648021.1 GI:34367180

Genbank record update date: Feb. 2, 2011 08:40 AM

### Cross-References

Sica G L., et al Immunity. 2003 June; 18(6):849-61

Other Information

Official Symbol: VTCN1

Other Aliases: RP11-229A19.4, B7-H4, B7H4, B7S1, B7X, B7h.5, PRO1291, VCTN1

Other Designations: B7 family member, H4; B7 superfamily member 1; T cell costimulatory molecule B7x; T-cell costimulatory molecule B7x; V-set domain-containing T-cell activation inhibitor 1; immune costimulatory protein B7-H4

(71) PTK7 (PTK7 Protein Tyrosine Kinase 7)

Nucleotide

Genbank accession no. AF447176

Genbank version no. AF447176.1 GI:17432420

Genbank record update date: Nov. 28, 2008 01:51 PM

Polypeptide

Genbank accession no. AAL39062

Genbank version no. AAL39062.1 GI:17432421

Genbank record update date: Nov. 28, 2008 01:51 PM

### Cross-References

Park S. K., et al J. Biochem. 119 (2), 235-239 (1996)

Other Information

Official Symbol: PTK7

Other Aliases: CCK-4, CCK4

Other Designations: colon carcinoma kinase 4; inactive tyrosine-protein kinase 7; pseudo tyrosine kinase receptor 7; tyrosine-protein kinase-like 7

(72) CD37 (CD37 Molecule)

Nucleotide

Genbank accession no. NM_001040031

Genbank version no. NM_001040031.1 GI:91807109

Genbank record update date: Jul. 29, 2012 02:08 PM

Polypeptide

Genbank accession no. NP_001035120

Genbank version no. NP_001035120.1 GI:91807110

Genbank record update date: Jul. 29, 2012 02:08 PM

### Cross-References

Schwartz-Albiez R., et al J. Immunol. 140 (3), 905-914 (1988)

Other Information

Official Symbol: CD37

Other Aliases: GP52-40, TSPAN26

Other Designations: CD37 antigen; cell differentiation antigen 37; leukocyte antigen CD37; leukocyte surface antigen CD37; tetraspanin-26; tspan-26

Antibodies

Boehringer Ingelheim: mAb 37.1 (Heider K H., et al Blood. 2011 Oct. 13; 118(15):4159-68)

Trubion: CD37-SMIP (G28-1 scFv-Ig) ((Zhao X., et al Blood. 2007; 110: 2569-2577)
For example, see US20110171208A1 SEQ ID NO: 253

Immunogen: K7153A (Deckert J., et al Cancer Res Apr. 15, 2012; 72(8 Supplement): 4625)

(73) CD138-SDC1 (Syndecan 1)

Nucleotide

Genbank accession no. AJ551176

Genbank version no. AJ551176.1 GI:29243141

Genbank record update date: Feb. 1, 2011 12:09 PM

Polypeptide

Genbank accession no. CAD80245

Genbank version no. CAD80245.1 GI:29243142

Genbank record update date: Feb. 1, 2011 12:09 PM

### Cross-References

O'Connell F P., et al Am J Clin Pathol. 2004 February; 121(2):254-63

Other Information

Official Symbol: SDC1

Other Aliases: CD138, SDC, SYND1, syndecan

Other Designations: CD138 antigen; heparan sulfate proteoglycan fibroblast growth factor receptor; syndecan proteoglycan 1; syndecan-1

Antibodies

Biotest: chimerized MAb (nBT062)—(Jagannath S., et al Poster ASH #3060, 2010; WIPO Patent Application WO/2010/128087)
For example, see US20090232810 SEQ ID NO: 1 and 2

Immunogen: B-B4 (Tassone P., et al Blood 104_3688-3696)
For example, see US20090175863A1 SEQ ID NO: 1 and 2

(74) CD74 (CD74 Molecule, Major Histocompatibility Complex, Class II Invariant Chain)

Nucleotide

Genbank accession no. NM_004355

Genbank version no. NM_004355.1 GI:343403784

Genbank record update date: Sep. 23, 2012 02:30 PM

Polypeptide

Genbank accession no. NP_004346

Genbank version no. NP_004346.1 GI:10835071

Genbank record update date: Sep. 23, 2012 02:30 PM

### Cross-References

Kudo, J., et al Nucleic Acids Res. 13 (24), 8827-8841 (1985)

Other Information

Official Symbol: CD74

Other Aliases: DHLAG, HLADG, II, Ia-GAMMA

Other Designations: CD74 antigen (invariant polypeptide of major histocompatibility complex, class II antigen-associated); HLA class II histocompatibility antigen gamma chain; HLA-DR antigens-associated invariant chain; HLA-DR-gamma; Ia-associated invariant chain; MHC HLA-DR gamma chain; gamma chain of class II antigens; p33

Antibodies

Immunomedics: hLL1 (Milatuzumab)—Berkova Z., et al Expert Opin Investig Drugs. 2010 January; 19(1):141-9)
For example, see US20040115193 SEQ ID NOs: 19, 20, 21, 22, 23 and 24

Genmab: HuMax-CD74 (see website)

(75) Claudins—CLs (Claudins)

### Cross-References

Offner S., et al Cancer Immunol Immunother. 2005 May; 54(5):431-45, Suzuki H., et al Ann N Y Acad Sci. 2012 July; 1258:65-70)

In humans, 24 members of the family have been described—see literature reference.

(76) EGFR (Epidermal Growth Factor Receptor)

Nucleotide

Genbank accession no. NM_005228

Genbank version no. NM_005228.3 GI:41927737

Genbank record update date: Sep. 30, 2012 01:47 PM

Polypeptide

Genbank accession no. NP_005219

Genbank version no. NP_005219.2 GI:29725609

Genbank record update date: Sep. 30, 2012 01:47 PM

### Cross-References

Dhomen N S., et al Crit Rev Oncog. 2012; 17(1):31-50

Other Information

Official Symbol: EGFR

Other Aliases: ERBB, ERB1, HER1, PIG61, mENA

Other Designations: avian erythroblastic leukemia viral (v-erb-b) oncogene homolog; cell growth inhibiting protein 40; cell proliferation-inducing protein 61; proto-oncogene c-ErbB-1; receptor tyrosine-protein kinase erbB-1

Antibodies

BMS: Cetuximab (Erbitux)—Broadbridge V T., et al Expert Rev Anticancer Ther. 2012 May; 12(5):555-65.
For example, see U.S. Pat. No. 6,217,866—ATTC deposit No. 9764.

Amgen: Panitumumab (Vectibix)—Argiles G., et al Future Oncol. 2012 April; 8(4):373-89
For example, see U.S. Pat. No. 6,235,883 SEQ ID NOs: 23-38.

Genmab: Zalutumumab—Rivera F., et al Expert Opin Biol Ther. 2009 May; 9(5):667-74.

YM Biosciences: Nimotuzumab—Ramakrishnan M S., et al MAbs. 2009 January-February; 1(1):41-8.
For example, see U.S. Pat. No. 5,891,996 SEQ ID NOs: 27-34.

(77) Her3 (ErbB3)-ERBB3 (v-erb-b2 Erythroblastic Leukemia Viral Oncogene Homolog 3 (Avian))

Nucleotide

Genbank accession no. M34309

Genbank version no. M34309.1 GI:183990

Genbank record update date: Jun. 23, 2010 08:47 PM

Polypeptide

Genbank accession no. AAA35979

Genbank version no. AAA35979.1 GI:306841

Genbank record update date: Jun. 23, 2010 08:47 PM

### Cross-References

Plowman, G. D., et al., Proc. Natl. Acad. Sci. U.S.A. 87 (13), 4905-4909 (1990)

Other Information

Official Symbol: ERBB3

Other Aliases: ErbB-3, HER3, LCCS2, MDA-BF-1, c-erbB-3, c-erbB3, erbB3-S, p180-ErbB3, p45-sErbB3, p85-sErbB3

Other Designations: proto-oncogene-like protein c-ErbB-3; receptor tyrosine-protein kinase erbB-3; tyrosine kinase-type cell surface receptor HER3

Antibodies

Merimack Pharma: MM-121 (Schoeberl B., et al Cancer Res. 2010 Mar. 15; 70(6):2485-2494)
For example, see US2011028129 SEQ ID NOs: 1, 2, 3, 4, 5, 6, 7 and 8.

(78) RON-MST1R (Macrophage Stimulating 1 Receptor (c-Met-Related Tyrosine Kinase))

Nucleotide

Genbank accession no. X70040

Genbank version no. X70040.1 GI:36109

Genbank record update date: Feb. 2, 2011 10:17 PM

Polypeptide

Genbank accession no. CAA49634

Genbank version no. CAA49634.1 GI:36110

Genbank record update date: Feb. 2, 2011 10:17 PM

### Cross-References

Ronsin C., et al Oncogene 8 (5), 1195-1202 (1993)

Other Information

Official Symbol: MST1R

Other Aliases: CD136, CDw136, PTK8, RON

Other Designations: MSP receptor; MST1R variant RON30; MST1R variant RON62; PTK8 protein tyrosine kinase 8; RON variant E2E3; c-met-related tyrosine kinase; macrophage-stimulating protein receptor; p185-Ron; soluble RON variant 1; soluble RON variant 2; soluble RON variant 3; soluble RONvariant 4

(79) EPHA2 (EPH Receptor A2)

Nucleotide

Genbank accession no. BC037166

Genbank version no. BC037166.2 GI:33879863

Genbank record update date: Mar. 6, 2012 01:59 PM

Polypeptide

Genbank accession no. AAH37166

Genbank version no. AAH37166.1 GI:22713539

Genbank record update date: Mar. 6, 2012 01:59 PM

### Cross-References

Strausberg R. L., et al Proc. Natl. Acad. Sci. U.S.A. 99 (26), 16899-16903 (2002)

Other Information

Official Symbol: EPHA2

Other Aliases: ARCC2, CTPA, CTPP1, ECK

Other Designations: ephrin type-A receptor 2; epithelial cell receptor protein tyrosine kinase; soluble EPHA2 variant 1; tyrosine-protein kinase receptor ECK

Antibodies

Medimmune: 1C1 (Lee J W., et al Clin Cancer Res. 2010 May 1; 16(9):2562-2570)
For example, see US20090304721A1 FIGS. 7 and 8.

(80) CD20-MS4A1 (Membrane-Spanning 4-Domains, Subfamily A, Member 1)

Nucleotide

Genbank accession no. M27394

Genbank version no. M27394.1 GI:179307

Genbank record update date: Nov. 30, 2009 11:16 AM

Polypeptide

Genbank accession no. AAA35581

Genbank version no. AAA35581.1 GI:179308

Genbank record update date: Nov. 30, 2009 11:16 AM

### Cross-References

Tedder T. F., et al Proc. Natl. Acad. Sci. U.S.A. 85 (1), 208-212 (1988)

Other Information

Official Symbol: MS4A1

Other Aliases: B1, Bp35, CD20, CVID5, LEU-16, MS4A2, S7

Other Designations: B-lymphocyte antigen CD20; B-lymphocyte cell-surface antigen E1; CD20 antigen; CD20 receptor; leukocyte surface antigen Leu-16

Antibodies

Genentech/Roche: Rituximab—Abdulla N E., et al BioDrugs. 2012 Apr. 1; 26(2):71-82.
For example, see U.S. Pat. No. 5,736,137, ATCC deposit No. HB-69119.

GSK/Genmab: Ofatumumab—Nightingale G., et al Ann Pharmacother. 2011 October; 45(10):1248-55.
For example, see US20090169550A1 SEQ ID NOs: 2, 4 and 5.

Immunomedics: Veltuzumab—Goldenberg D M., et al Leuk Lymphoma. 2010 May; 51(5):747-55.
For example, see U.S. Pat. No. 7,919,273B2 SEQ ID NOs: 1, 2, 3, 4, 5 and 6.

(81) Tenascin C—TNC (Tenascin C)

Nucleotide

Genbank accession no. NM_002160

Genbank version no. NM_002160.3 GI:340745336

Genbank record update date: Sep. 23, 2012 02:33 PM

Polypeptide

Genbank accession no. NP_002151

Genbank version no. NP_002151.2 GI:153946395

Genbank record update date: Sep. 23, 2012 02:33 PM

### Cross-References

Nies D. E., et al J. Biol. Chem. 266 (5), 2818-2823 (1991); Siri A., et al Nucleic Acids Res. 19 (3), 525-531 (1991)

Other Information

Official Symbol: TNC

Other Aliases: 150-225, GMEM, GP, HXB, JI, TN, TN-C

Other Designations: GP 150-225; cytotactin; glioma-associated-extracellular matrix antigen; hexabrachion (tenascin); myotendinous antigen; neuronectin; tenascin; tenascin-C isoform 14/AD1/16

Antibodies

Philogen: G11 (von Lukowicz T., et al J Nucl Med. 2007 April; 48(4):582-7) and F16 (Pedretti M., et al Lung Cancer. 2009 April; 64(1):28-33)
For example, see U.S. Pat. No. 7,968,685 SEQ ID NOs: 29, 35, 45 and 47.

(82) FAP (Fibroblast Activation Protein, Alpha)

Nucleotide

Genbank accession no. U09278

Genbank version no. U09278.1 GI:1888315

Genbank record update date: Jun. 23, 2010 09:22 AM

Polypeptide

Genbank accession no. AAB49652

Genbank version no. AAB49652.1 GI:1888316

Genbank record update date: Jun. 23, 2010 09:22 AM

### Cross-References

Scanlan, M. J., et al Proc. Natl. Acad. Sci. U.S.A. 91 (12), 5657-5661 (1994)

Other Information

Official Symbol: FAP

Other Aliases: DPPIV, FAPA

Other Designations: 170 kDa melanoma membrane-bound gelatinase; integral membrane serine protease; seprase

(83) DKK-1 (Dickkopf 1 Homolog (Xenopus laevis)

Nucleotide

Genbank accession no. NM_012242

Genbank version no. NM_012242.2 GI:61676924

Genbank record update date: Sep. 30, 2012 01:48 PM

Polypeptide

Genbank accession no. NP_036374

Genbank version no. NP_036374.1 GI:7110719

Genbank record update date: Sep. 30, 2012 01:48 PM

### Cross-References

Fedi P. et al J. Biol. Chem. 274 (27), 19465-19472 (1999)

Other Information

Official Symbol: DKK1

Other Aliases: UNQ492/PRO1008, DKK-1, SK

Other Designations: dickkopf related protein-1; dickkopf-1 like; dickkopf-like protein 1; dickkopf-related protein 1; hDkk-1

Antibodies

Novartis: BHQ880 (Fulciniti M., et al Blood. 2009 Jul. 9; 114(2):371-379)
For example, see US20120052070A1 SEQ ID NOs: 100 and 108.

(84) CD52 (CD52 Molecule)

Nucleotide

Genbank accession no. NM_001803

Genbank version no. NM_001803.3 GI:1519245483

Genbank record update date: May 1, 2019 02:13 AM

Polypeptide

Genbank accession no. NP_001794

Genbank version no. NP_001794.2 GI:68342030

Genbank record update date: May 1, 2019 02:13 AM

### Cross-References

Xia M. Q., et al Eur. J. Immunol. 21 (7), 1677-1684 (1991)

Other Information

Official Symbol: CD52

Other Aliases: CDW52

Other Designations: CAMPATH-1 antigen; CD52 antigen (CAMPATH-1 antigen); CDW52 antigen (CAMPATH-1 antigen); cambridge pathology 1 antigen; epididymal secretory protein E5; he5; human epididymis-specific protein 5

Antibodies

Alemtuzumab (Campath)—Skoetz N., et al Cochrane Database Syst Rev. 2012 Feb. 15; 2:CD008078.
For example, see Drugbank Acc. No. DB00087 (BIOD00109, BTD00109)

(85) CS1-SLAMF7 (SLAM Family Member 7)

Nucleotide

Genbank accession no. NM_021181

Genbank version no. NM_021181.3 GI:1993571

Genbank record update date: Jun. 29, 2012 11:24 AM

Polypeptide

Genbank accession no. NP_067004

Genbank version no. NP_067004.3 GI:19923572

Genbank record update date: Jun. 29, 2012 11:24 AM

### Cross-References

Boles K. S., et al Immunogenetics 52 (3-4), 302-307 (2001)

Other Information

Official Symbol: SLAMF7

Other Aliases: UNQ576/PRO1138, 19A, CD319, CRACC, CS1

Other Designations: 19A24 protein; CD2 subset 1; CD2-like receptor activating cytotoxic cells; CD2-like receptor-activating cytotoxic cells; membrane protein FOAP-12; novel LY9 (lymphocyte antigen 9) like protein; protein 19A

Antibodies

BMS: elotuzumab/HuLuc63 (Benson D M., et al J Clin Oncol. 2012 Jun. 1; 30(16):2013-2015)
For example, see US20110206701 SEQ ID NOs: 9, 10, 11, 12, 13, 14, 15 and 16.

(86) Endoglin—ENG (Endoglin)

Nucleotide

Genbank accession no. AF035753

Genbank version no. AF035753.1 GI:3452260

Genbank record update date: Mar. 10, 2010 06:36 PM

Polypeptide

Genbank accession no. AAC32802

Genbank version no. AAC32802.1 GI:3452261

Genbank record update date: Mar. 10, 2010 06:36 PM

### Cross-References

Rius C., et al Blood 92 (12), 4677-4690 (1998)

Official Symbol: ENG

Other Information

Other Aliases: RP11-228B15.2, CD105, END, HHT1, ORW, ORW1

Other Designations: CD105 antigen

(87) Annexin A1—ANXA1 (Annexin A1)

Nucleotide

Genbank accession no. X05908

Genbank version no. X05908.1 GI:34387

Genbank record update date: Feb. 2, 2011 10:02 AM

Polypeptide

Genbank accession no. CAA29338

Genbank version no. CAA29338.1 GI:34388

Genbank record update date: Feb. 2, 2011 10:02 AM

### Cross-References

Wallner B. P., et al Nature 320 (6057), 77-81 (1986)

Other Information

Official Symbol: ANXA1

Other Aliases: RP11-71A24.1, ANX1, LPC1

Other Designations: annexin I (lipocortin I); annexin-1; calpactin 1l; calpactin-2; chromobindin-9; lipocortin I; p35; phospholipase A2 inhibitory protein

(88) V-CAM (CD106)-VCAM1 (Vascular Cell Adhesion Molecule 1)

Nucleotide

Genbank accession no. M60335

Genbank version no. M60335.1 GI:340193

Genbank record update date: Jun. 23, 2010 08:56 AM

Polypeptide

Genbank accession no. AAA61269

Genbank version no. AAA61269.1 GI:340194

Genbank record update date: Jun. 23, 2010 08:56 AM

### Cross-References

Hession C., et al J. Biol. Chem. 266 (11), 6682-6685 (1991)

Other Information

Official Symbol VCAM1

Other Aliases: CD106, INCAM-100

Other Designations: CD106 antigen; vascular cell adhesion protein 1

(89) DLK-1 (Protein Delta Homolog 1)

Nucleotide

Genbank accession no. Z12172

Genbank version no. Z12172.1

Genbank record update date: Feb. 2, 2011 10:34 AM

Polypeptide

Genbank accession no. CAA78163

Genbank version no. CAA78163.1

Genbank record update date: Feb. 2, 2011 10:34 AM

### Cross-References

Laborda J. et al., J. Biol. Chem. 268:3817-3820(1993)

Other Information

Official Symbol DLK-1

Other Aliases: pG2

Other Designations: cleaved into Fetal antigen 1, FA1

(90) KAAG1 (Kidney-Associated Antigen 1)

Nucleotide

Genbank accession no. AF181720

Genbank version no. AF181720.1

Genbank record update date: Jul. 26, 2016 05:57 AM

Polypeptide

Genbank accession no. AAF23611

Genbank version no. AAF23611.1

Genbank record update date: Jul. 26, 2016 05:57 AM

### Cross-References

Van den Eynde B. J. et al., J. Exp. Med. 190:1793-1800(1999)

Other Information

Official Symbol KAAG1

Other Aliases: RU2 antisense gene protein

(91) IL13RA2

Nucleotide

Genbank accession no. X95302

Genbank version no. X95302.1

Genbank record update date: Feb. 2, 2011 10:44 AM

Polypeptide

Genbank accession no. CAA64617

Genbank version no. CAA64617.1

Genbank record update date: Feb. 2, 2011 10:44 AM

Uniprot accession no. Q14627-1

### Cross-References

Caput D., Laurent P., Kaghad M., Lelias J. M., Lefort S., Vita N., Ferrara P., J. Biol. Chem. 271:16921-16926(1996)

Other Information

Official Symbol: IL13RA2

Other Aliases: IL13R, CD213a

(92) Endosialin

Nucleotide

Genbank accession no. AF279142

Genbank version no. AF279142.1

Genbank record update date: Mar. 10, 2010 10:42 PM

Polypeptide

Genbank accession no. AAG00867

Genbank version no. AAG00867.1

Genbank record update date: Mar. 10, 2010 10:42 PM

Uniprot accession no. Q9HCU0-1

### Cross-References

St Croix B., Rago C., Velculescu V. E., Traverso G., Romans K. E., Montgomery E., Lal A., Riggins G. J., Lengauer C., Vogelstein B., Kinzler K. W., Science 289:1197-1202(2000)

Other Information

Official Symbol: Endosialin

Other Aliases: CD248, TEM1, CD164L1

(93) CD48

Nucleotide

Genbank accession no. M59904

Genbank version no. M59904.1

Genbank record update date: Jun. 23, 2010 08:47 AM

Polypeptide

Genbank accession no. AAA62834

Genbank version no. AAA62834.1

Genbank record update date: Jun. 23, 2010 08:47 AM

Uniprot accession no. P09326-1

### Cross-References

Vaughan H. A., Henning M. M., Purcell D. F. J., McKenzie I. F. C., Sandrin M. S., Immunogenetics 33:113-117(1991)

Other Information

Official Symbol: CD48

Other Aliases: BLAST-1, BCM1, MEM-102, SLAMF2, TCT1

(94) LRRC15

Nucleotide

Genbank accession no. AB071037

Genbank version no. AB071037.1

Genbank record update date: Oct. 11, 2008 01:23 AM

Polypeptide

Genbank accession no. BAB84587

Genbank version no. BAB84587.1

Genbank record update date: Oct. 11, 2008 01:23 AM

Uniprot accession no. Q8TF66-1

### Cross-References

Satoh K., Hata M., Yokota H., Biochem. Biophys. Res. Commun. 290:756-762(2002)

Other Information

Official Symbol: LRRC15

Other Aliases: LIB

(95) SLAMF6

Nucleotide

Genbank accession no. AJ277141

Genbank version no. AJ277141.1

Genbank record update date: Feb. 1, 2011 10:36 AM

Polypeptide

Genbank accession no. CAC59749

Genbank version no. CAC59749.1

Genbank record update date: Feb. 1, 2011 10:36 AM

Uniprot accession no. Q96DU3-1

### Cross-References

Bottino C., Falco M., Parolini S., Marcenaro E., Augugliaro R., Sivori S., Landi E., Biassoni R., Notarangelo L. D., Moretta L., Moretta A., J. Exp. Med. 194:235-246(2001)

Other Information

Official Symbol: SLAMF6

Other Aliases: CD352, NTB-A

(96) PLAC1

Nucleotide

Genbank accession no. AF234654

Genbank version no. AF234654.1

Genbank record update date: Nov. 16, 2010 12:54 PM

Polypeptide

Genbank accession no. AAG22596

Genbank version no. AAG22596.1

Genbank record update date: Nov. 16, 2010 12:54 PM

Uniprot accession no. Q9HBJ0-1

### Cross-References

Cocchia M., Huber R., Pantano S., Chen E. Y., Ma P., Forabosco A., Ko M. S. H., Schlessinger D., Genomics 68:305-312(2000)

Other Information

Official Symbol: PLAC1

Other Aliases: n/a

Fc Fusion Proteins

In some embodiments the cell-binding agent is a Fc fusion protein.

The term “Fc fusion protein” is used herein to refer to a fusion protein comprising an immunoglobin Fc domain fused to another peptide. The fused peptide may be any other proteinaceous molecule of interest, such as a binding moiety, a ligand that activates upon interaction with a cell-surface receptor, a peptidic antigen against a challenging pathogen, or a ‘bait’ protein to identify binding partners assembled in a protein microarray.

Typically the fused partners have significant therapeutic potential, and they are attached to an Fc-domain to endow the fusions with a number of additional beneficial biological and pharmacological properties. For example, the presence of the Fc domain typically markedly increases their plasma half-life, which prolongs therapeutic activity. From a biophysical perspective, the Fc domain folds independently and can improve the solubility and stability of the fused peptide both in vitro and in vivo. From a technological viewpoint, the Fc region allows for easy cost-effective purification by protein-G/A affinity chromatography during manufacture.

In the context of the present disclosure, the Fc domain will typically also bear an N-linked glycan which can be modified and conjugates to form a clycoconjugate as described herein. Accordingly, the use of a Fc fusion as the CBA provides an elegant method of forming a glycoconjugate comprising the payloads described herein conjugated to a proteinaceous molecule of interest that in its non Fc-fusion form does not comprise a suitable N-linked glycan.

As for the antibodies described below, depending on the amino acid sequence of the constant domain of their heavy chains, Fc domains can be assigned to different “classes.” There are five major antibody classes form which Fc domains are derived: IgA, IgD, IgE, IgG, and IgM, and several of these may be further divided into “subclasses” (isotypes), e.g., IgG1, IgG2, IgG3, IgG4, IgA, and IgA2. The IgG isotype is preferred, in particular the IgG1 sub-type. The heavy-chain constant domains that correspond to the different classes of antibodies are called α, δ, ε, γ, and μ, respectively. The subunit structures and three-dimensional configurations of different classes of immunoglobulins are well known.

Antibodies

In some embodiments the cell-binding agent is an antibody.

The term “antibody” herein is used in the broadest sense and specifically covers monoclonal antibodies, polyclonal antibodies, dimers, multimers, multispecific antibodies {e.g., bispecific antibodies), and antibody fragments, so long as they exhibit the desired biological activity (Miller et al (2003) Jour, of Immunology 170:4854-4861). Antibodies may be murine, human, humanized, chimeric, or derived from other species. An antibody is a protein generated by the immune system that is capable of recognizing and binding to a specific antigen. (Janeway, C, Travers, P., Walport, M., Shlomchik (2001) ImmunoBiology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)2, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising a VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1 q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The IgG isotype is preferred, in particular the IgG1 sub-type. The heavy-chain constant domains that correspond to the different classes of antibodies are called α, δ, ε, γ, and μ, respectively. The subunit structures and three-dimensional configurations of different classes of immunoglobulins are well known.

Techniques to reduce the in vivo immunogenicity of a non-human antibody or antibody fragment include those termed “humanisation”.

A “humanized antibody” refers to a polypeptide comprising at least a portion of a modified variable region of a human antibody wherein a portion of the variable region, preferably a portion substantially less than the intact human variable domain, has been substituted by the corresponding sequence from a non-human species and wherein the modified variable region is linked to at least another part of another protein, preferably the constant region of a human antibody. The expression “humanized antibodies” includes human antibodies in which one or more complementarity determining region (“CDR”) amino acid residues and/or one or more framework region (“FW” or “FR”) amino acid residues are substituted by amino acid residues from analogous sites in rodent or other non-human antibodies. The expression “humanized antibody” also includes an immunoglobulin amino acid sequence variant or fragment thereof that comprises an FR having substantially the amino acid sequence of a human immunoglobulin and a CDR having substantially the amino acid sequence of a non-human immunoglobulin.

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g. murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins.

There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

The antibody may be an intact antibody. The antibody may be humanised, deimmunised or resurfaced. The antibody may be a fully human monoclonal IgG1 antibody, preferably IgG1,κ.

Numbering of Amino Acid Positions in Immunoglobulin (Ig) Molecules

The numbering of the amino acids used herein is according to the numbering system of the EU index as set forth in Kabat et al. (1991, NIH Publication 91-3242, National Technical Information Service, Springfield, VA, hereinafter “Kabat”). The “EU index as set forth in Kabat” refers to the residue numbering of the human IgG 1 EU antibody as described in Kabat et al. supra.

In the case of substitutions in, for example, IgG2, IgG3, and IgG4 (or of IgA1, IgA2, IgD, IgE, IgM etc.) the skilled person can readily use sequence alignment programs such as NCBI BLAST® (http://blast.ncbi.nlm.nih.gov/Blast.cgi) to align the sequences with IgG1 to determine which residues of the desired isoform correspond to the Kabat positions described herein.

In some embodiments the payload is conjugated to the N-linked glycan attached to an asparagine residue located at the position corresponding to 297 of IgG1 according to the EU index as set forth in Kabat.

In some embodiments the antibody is an intact antibody having 2 N-linked glycans bearing Sd(A^{P})ₓ moieties (ie. y=2). In some embodiments the antibody has exactly 2 N-linked glycans bearing Sd(A^{P})ₓ moieties.

Saccharides

The general terms “sugar”, “sugar residue”, “sugar moiety”, and “saccharide” are used interchangeably herein used to indicate a monosaccharide, for example glucose (Glc), galactose (Gal), mannose (Man) and fucose (Fuc).

The term “Sug” is used in the general formula herein to designate an otherwise unspecified sugar moiety. Typically, “Sug” in the structures disclosed herein represents a sugar moiety optionally present (ie. b=1 or 0) on the GlcNAc residue that is directly bound to the CBA via its C1 carbon. Typically Sug is linked to the GlcNAc via glycosidic bond to the GlcNAc C6, preferably in an α1-6 configuration. Typically Sug is a fucose moiety. In some embodiments the GlcNAc bears α1-6 fucose. In some embodiments the fucose moiety has the structure:

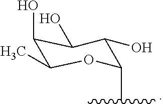 [SMILES: *C1OC(C)C(O)C(O)C1O]

In some embodiments, the compositions comprise glycoconjugates where at least 90%, at least 95%, at least 98%, or at least 99% of the glycoconjugates have a Sug conjugated to the GlcNAc C6. In some embodiments, the compositions comprise glycoconjugates where at least 90%, at least 95%, at least 98%, or at least 99% of the glycoconjugates have a hydroxyl group at the GlcNAc C6 (ie. there is no sug conjugated to the GLcNAc C6).

Sugar Derivative

The term “sugar derivative” is used herein to indicate a derivative of a monosaccharide sugar, i.e. a monosaccharide sugar comprising substituents and/or functional groups. Examples of a sugar derivative include amino sugars and sugar acids, e.g. glucosamine (GlcN), galactosamine (GalN), Neuraminic acid (NeuN), N-acetylglucosamine (GlcNAc), N-acetylgalactosamine (GalNAc), N-acetylneuraminic acid (NeuNAc) and N-acetylmuramic acid (MurNAc), glucuronic acid (GlcA), and iduronic acid (IdoA).

As described elsewhere herein, the glycoconjugates are typically produced and/or modified using enzyme catalysed processes. Accordingly, the sugar derivatives described herein (eg. “GlcNAc”, “Sug”, “Gal”) typically have the properties and configuration that allows for their efficient use by the enzyme catalysts. Preferably the usage derivatives such as “GlcNAc”, “Sug”, and “Gal” described herein are ‘D’ enantiomers.

The term “sugar derivative” is also used herein to indicate compounds herein described with the label “Sd(A^{F/P})ₓ”, wherein Sd is a sugar or a sugar derivative, and wherein Sd comprises x groups A^{F/P}. A^{F/P} may denote either unconjugated functional groups (A^{F}) or, post-conjugation, the conjugated payloads (A^{P}) bonded to Sd.

In some embodiments Sd(A^{F/P})ₓ comprises 1, 2, 3, or 4 groups A^{F/P}.

Sialic Acid Derivative

In some preferred embodiments Sd(A^{P})ₓ is a sialic acid derivative, wherein “sialic acid” is a generic term for N- and/or O-substituted derivatives of NeuN, such as Neu5Ac (NeuN acylated on the amine group found on C5).

In some embodiments of the glycoconjugates described herein, the sialic acid derivative has the formula:

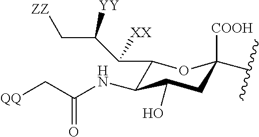

wherein:
QQ is hydrogen or a conjugated payload;
ZZ is hydroxyl or a conjugated payload;
YY is hydroxyl or a conjugated payload; and/or
XX is hydroxyl or a conjugated payload;

and wherein at least one of QQ, ZZ, YY, and XX is a conjugated payload.

In some embodiments of the glycosylated cell-binding agents, the sialic acid derivative has the formula:

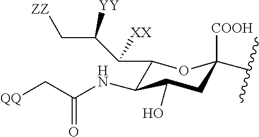

wherein:
QQ is hydrogen or a functional group A;
ZZ is hydroxyl or a functional group A;
YY is hydroxyl or a functional group A; and/or
XX is hydroxyl or a functional group A;

and wherein at least one of QQ, ZZ, YY, and XX is a functional group A.

Payload

The payload is a ‘PBD payload’. That is, the payload is, comprises, or releases upon metabolism a PBD compound, as defined below in the section entitled “PBD compound”.

The conjugate chemistry described herein above allows the glycosylated cell-binding agents described herein to be conjugated to a wide-range of PBD payloads. A preferred class of PBD payload comprise a PBD drug moiety, with the conjugation of the drug to the cell-binding agent allowing the PBD drug to be delivered to the bound target cell with a high degree of precision.

Conjugated Drug-Linkers

As noted above the conjugated payload comprises, or releases upon metabolism, a PBD compound. The conjugated payload may comprise, or releases upon metabolism, multiple PBD compounds. In some embodiments one or more of the PBD compounds is linked to the sugar derivative (Sd) via a linker.

In preferred embodiments, the PBD drug moiety is conjugated to the glycosylated cell-binding agents described herein via a linker moiety (a so-called ‘PBD drug-linker’ payload) to yield a conjugate having the formula:

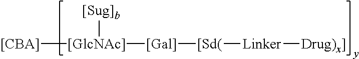

wherein:
CBA is a Cell Binding Agent;
GlcNAc is a N-acetyl-glucosamine moiety;
Sug is a sugar moiety, wherein b=1 or 0;
Gal is a galactose moiety;
Sd(-Linker-Drug)ₓ is a sugar derivative comprising x conjugated PBD drug-linkers,
wherein x=1, 2, 3, or 4; and
wherein y=1 to 20.

In some embodiments, multiple PBD drug moities can be conjugated to the same linker that is conjugated to Sd, the resultant conjugates having the formula:

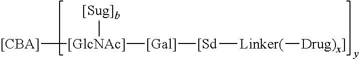

In some embodiments, multiple PBD drug moities can be conjugated to the same linker, and multiple linkers can be conjugated to Sd, the resultant conjugates having the formula:

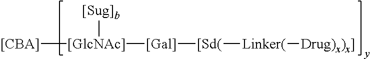

Accordingly, in some exemplary embodiments Sd(A^{P})ₓ has one functional group A^{P} that is a drug-linker payload at position QQ, thus:

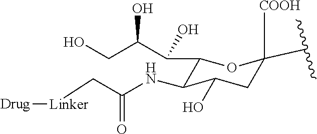

In some exemplary embodiments Sd(A^{P})ₓ has one functional group A^{P} that is a drug-linker payload at position ZZ, thus:

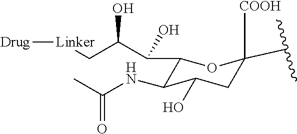

In yet further embodiments, the payload (e.g., drug) is linked to the sialoside at position QQ and position ZZ. The payload and linkers may be the same or different.

### PBD Drug-Linkers Embodiments

In some embodiments the conjugated PBD drug-linker payload has a formula selected from the group consisting of:

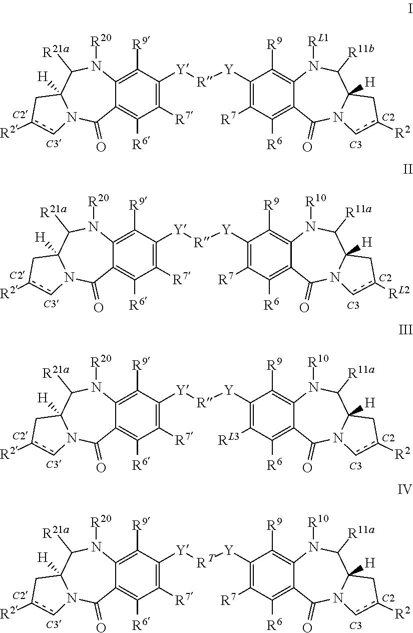

wherein:
R⁶ is selected from H, R, OH, OR, SH, SR, NH₂, NHR, NRR′, nitro, Me₃Sn and halo;
R^{6′} is selected from H, R, OH, OR, SH, SR, NH₂, NHR, NRR′, nitro, Me₃Sn and halo;
R⁹ is selected from H, R, OH, OR, SH, SR, NH₂, NHR, NRR′, nitro, Me₃Sn and halo;
R^{9′} is selected from H, R, OH, OR, SH, SR, NH₂, NHR, NRR′, nitro, Me₃Sn and halo;
Y is selected from O, S, or NH;
Y′ is selected from O, S, or NH;
when there is a double bond present between C2 and C3, R² is selected from the group consisting of:
(ia) C₅₋₁₀ aryl group, optionally substituted by one or more substituents selected from the group comprising: halo, nitro, cyano, hydroxy, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

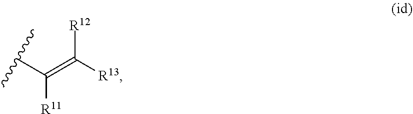

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

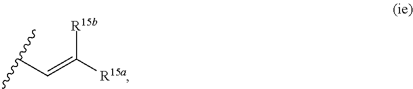

wherein one of R^{15a} and R^{15b} is H and the other is selected from: phenyl, which phenyl is optionally substituted by a group selected from halo, methyl, methoxy; pyridyl; and thiophenyl; and

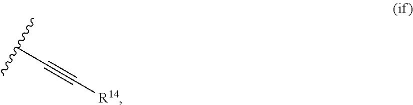

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2 and C3,
R² is selected from H, OH, F, diF and

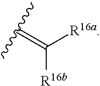

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester; when there is a double bond present between C2′ and C3′, R^{2′} is selected from the group consisting of:
(iia) C₅₋₁₀ aryl group, optionally substituted by one or more substituents selected from the group comprising: halo, nitro, cyano, hydroxy, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl;

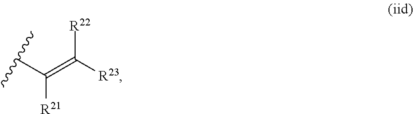

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

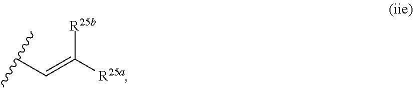

wherein one of R^{25a} and R^{25b} is H and the other is selected from: phenyl, which phenyl is optionally substituted by a group selected from halo, methyl, methoxy; pyridyl; and thiophenyl; and

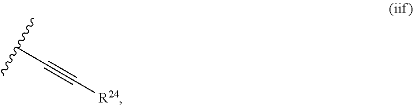

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R^{2′} is selected from H, OH, F, diF and

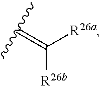

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁷ is selected from H, R, OH, OR, SH, SR, NH₂, NHR, NHRR′, nitro, Me₃Sn and halo;
R^{7′} is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
(a) R¹⁰ is H, and R^{11a} is OH or OR^{A}, where R^{A} is C₁₋₄ alkyl; or
(b) R¹⁰ and R^{11a} form a nitrogen-carbon double bond between the nitrogen and carbon atoms to which they are bound; or
(c) R¹⁰ is H and R^{11a} is SO_{z}M, where z is 2 or 3 and M is a monovalent pharmaceutically acceptable cation; or
(d) R¹⁰ is H and R^{11a} is H; or
(e) R^{11a} is OH or OR^{A}, where R^{A} is C₁₋₄ alkyl and R¹⁰ is selected from:

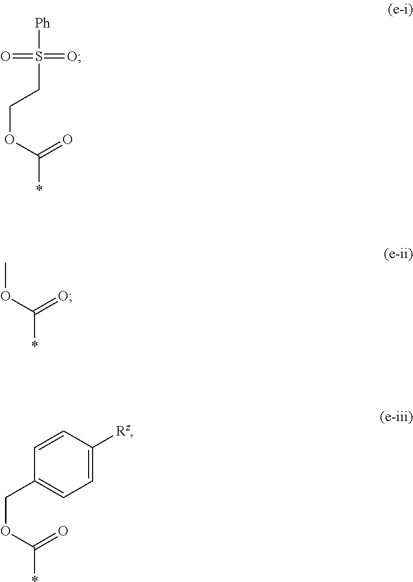

where R^{Z} is selected from:

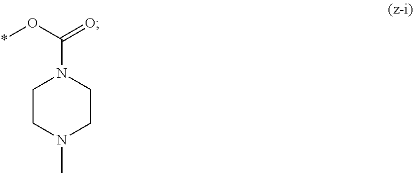 [SMILES: *OC(=O)N1CCN(C)CC1]

(z-ii) OC(═O)CH₃;
(z-iii) NO₂;
(z-iv) OMe;
(z-v) glucuronide;
   (z-vi) —C(═O)—X₁—NHC(═O)X₂—NH—R^{ZC}, where —C(═O)—X₁—NH— and —C(═O)—X₂—NH— represent natural amino acid residues and R^{ZC} is selected from Me, OMe, OCH₂CH₂OMe;
(a) R²⁰ is H, and R^{21a} is OH or OR^{A}, where R^{A} is C₁₋₄ alkyl; or
(b) R²⁰ and R^{21a} form a nitrogen-carbon double bond between the nitrogen and carbon atoms to which they are bound; or
(c) R²⁰ is H and R^{21a} is SO_{z}M, where z is 2 or 3 and M is a monovalent pharmaceutically acceptable cation; or
(d) R²⁰ is H and R^{21a} is H; or
(e) R^{21a} is OH or OR^{A}, where R^{A} is C₁₋₄ alkyl and R²⁰ is selected from:

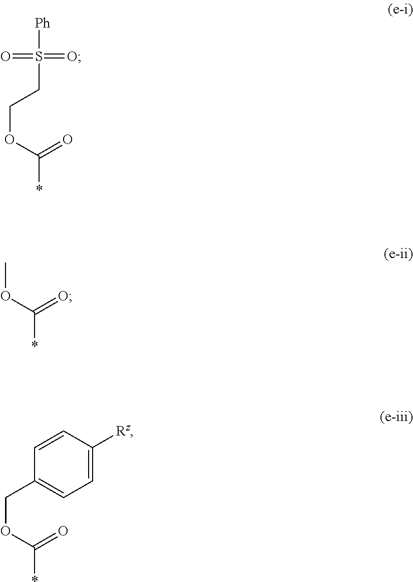

where R^{Z} is selected from:

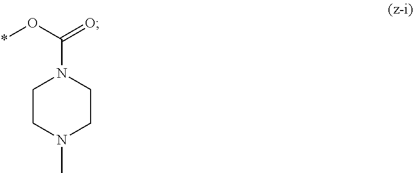 [SMILES: *OC(=O)N1CCN(C)CC1]

(z-ii) OC(═O)CH₃;
(z-iii) NO₂;
(z-iv) OMe;
(z-v) glucuronide;
(z-vi) —C(═O)—X₁—NHC(═O)X₂—NH—R^{ZC}, where —C(═O)—X₁—NH— and —C(═O)—X₂—NH— represent natural amino acid residues and R^{ZC} is selected from Me, OMe, OCH₂CH₂OMe;
R^{L1} and R^{11b}
R^{L1} is a linker for connection to Sd(A^{P})ₓ;
R^{11b} is OH or OR^{A}, where R^{A} is C₁₋₄ alkyl;
R^{L2}
R^{L2} is of formula IIIa, formula IIIb or formula IIIc:

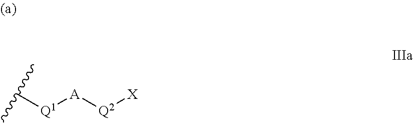

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

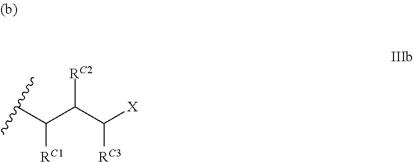

where;

R^{C1}, R^{C2} and R^{C3} are independently selected from H and unsubstituted C₁₋₂ alkyl;

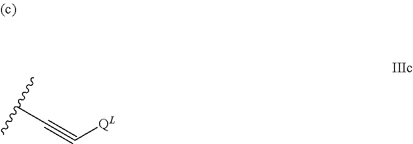

where Q^{L} is selected from O—R^{L2′}, S—R^{L2′} and NR^{N}—C(═O)—R^{L2′}, and R^{N} is selected from H, methyl and ethyl
X is selected from the group comprising: O—R^{L2′}, S—R^{L2′}, CO₂—R^{L2′}, CO—R^{L2′}, NR^{N}—C(═O)—R^{L2′}, NHNH—R^{L2′}, CONHNH—R^{L2′},

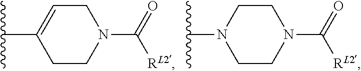

wherein R^{N} is selected from the group comprising H and C₁₋₄ alkyl;
R^{L2′} is a linker for connection to Sd(A^{P})ₓ;
R^{L3}
R^{L3} is selected from formulae A1, A2, A3, A4 and A5:

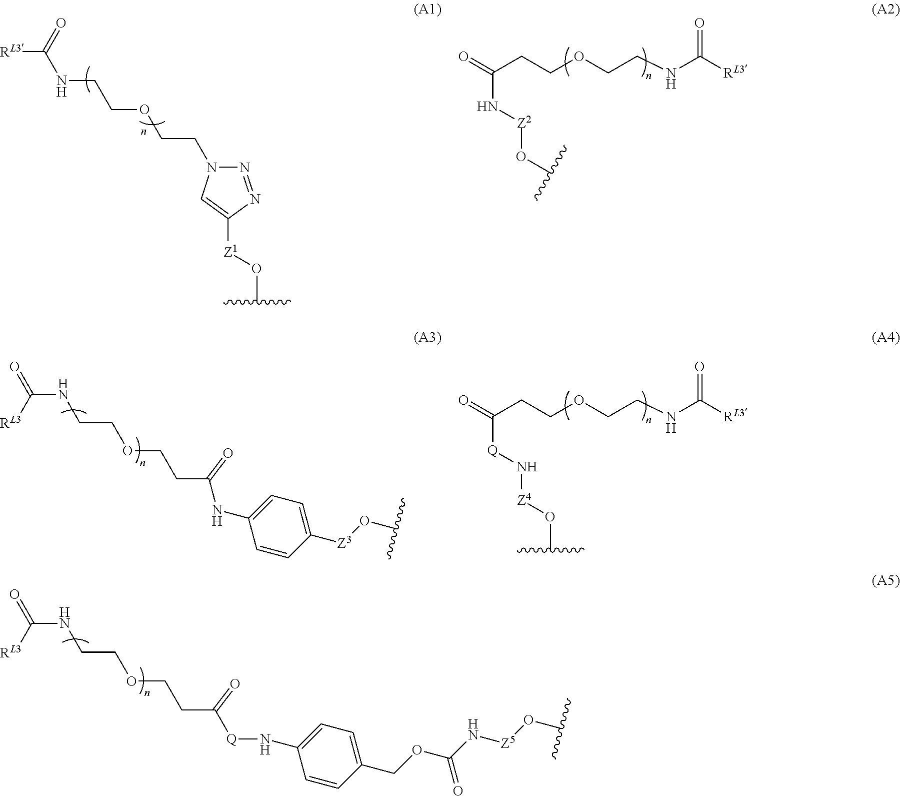

Z¹ is a C₁₋₃ alkylene group;
Z² is a C₁₋₃ alkylene group;
Z³ is a C₁₋₃ alkylene group;
Z⁴ is a C₁₋₃ alkylene group;
Z⁵ is a C₁₋₃ alkylene group;
n is an integer between 0 and 48;
Q is:

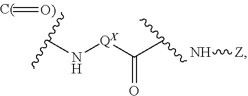

where Q^{X} is such that Q is an amino-acid residue, a dipeptide residue, a tripeptide residue, or a non-peptide moiety defined as PM in WO2015/095124;
R^{L3′} is a linker for connection to Sd(A^{P})ₓ;
R^{T}
R^{T} is:

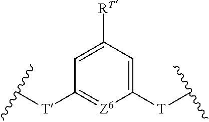

wherein
T and T″ are independently selected from a single bond or a C₁₋₉ alkylene, which chain may be interrupted by one or more heteroatoms e.g. O, S, N(H), NMe, provided that the number of atoms in the shortest chain of atoms between Y and Y′ is 3 to 12 atoms;
Z⁶ is CH or N;
R^{T′} is selected from formulae B1, B2, B3, B4, B5, B6, and B7:

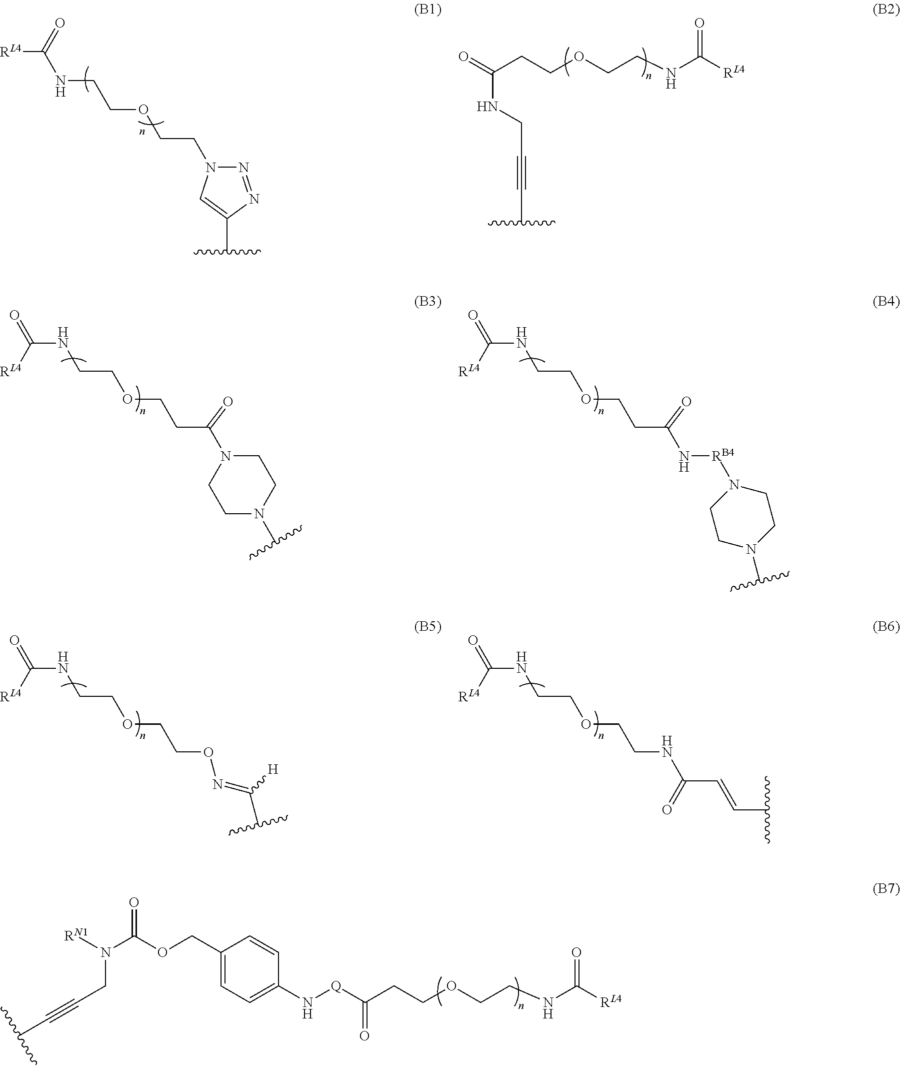

n is an integer selected in the range of 0 to 48;
R^{B4} is a C₁₋₆ alkylene group;
Q is:

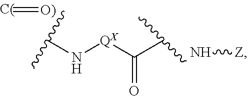

where Q^{X} is such that Q is an amino-acid residue, a dipeptide residue, a tripeptide residue, or a non-peptide moiety defined as PM in WO2015/095124;
R^{L4} is a linker for connection to Sd(A^{P})ₓ.

Another preferred class of linker for connection to the Sd(A^{P})ₓ is a linker of formula Z1 or Z2:

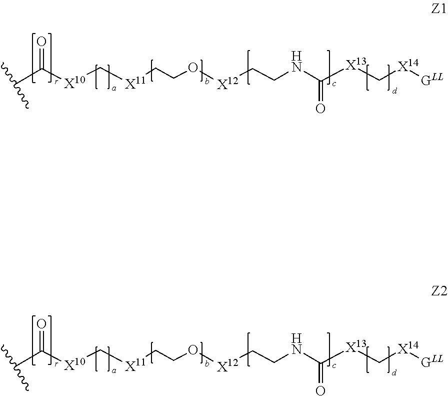

where r=0 or 1, a=0 to 5, b=0 to 16, c=0 or 1, d=0 to 5, G^{LL} is a linking moiety, and one of X¹⁰, X¹¹, X¹², X¹³ and X¹⁴ may be selected from:

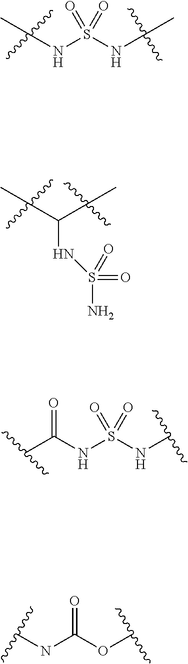 [SMILES: *NS(=O)(=O)N* ; *C(*)NS(N)(=O)=O ; *NS(=O)(=O)NC(*)=O ; *NC(=O)O*]

the remainder being a single bond.

G^{LL} is selected from:

| | |
|---|---|
| (G^{LL8-1}) | 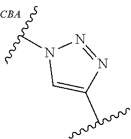 [SMILES: *c1cn(*)nn1] |
| (G^{LL8-2}) | 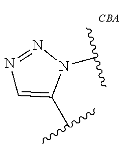 [SMILES: *c1cnnn1*] |
| (G^{LL9-1}) | 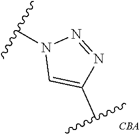 [SMILES: *c1cn(*)nn1] |
| (G^{LL9-2}) | 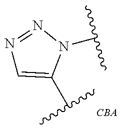 [SMILES: *c1cnnn1*] |
| G^{LL10} | 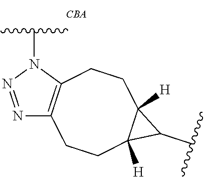 [SMILES: *C1[C@H]2CCc3c(nnn3*)CC[C@@H]12] |
| G^{LL11} | 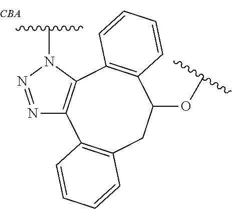 [SMILES: *OC1Cc2ccccc2-c2nnn(*)c2-c2ccccc21] |
| G^{LL12} | 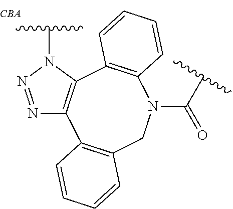 [SMILES: *C(=O)N1Cc2ccccc2-c2nnn(*)c2-c2ccccc21] |
| G^{LL13} | 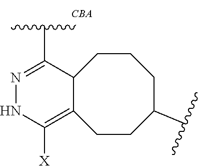 |
| G^{LL14} | 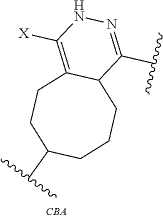 |

where CBA indicates where the group is bound to Sd(A^{P})ₓ.

### Linker Embodiments

In some preferred embodiments the linker moiety comprises an ionizable group such as:
an acidic group/moiety, such as, for example, —CO₂H, —NHSO₂NH₂, —NHSO₂NHR where R is an alkyl moiety, —SO₃H, sialic acid, glutamic acid, a glutamic acid sidechain, aspartic acid, an aspartic acid sidechain, and the like, and salts or ionic groups/moieties formed therefrom; or
a basic group/moiety, such as, for example, an amine group/moiety (e.g., a primary amine group, a secondary amine moiety, or a tertiary amine moiety), guanidinium group, and the like, and salts or ionic groups/moieties formed therefrom;

with the proviso that no carbonyl is adjacent (e.g., alpha) to a —NHSO₂NH— moiety or —NHSO₂NH₂ group.

In some embodiments the drug-linker has a structure chosen from:

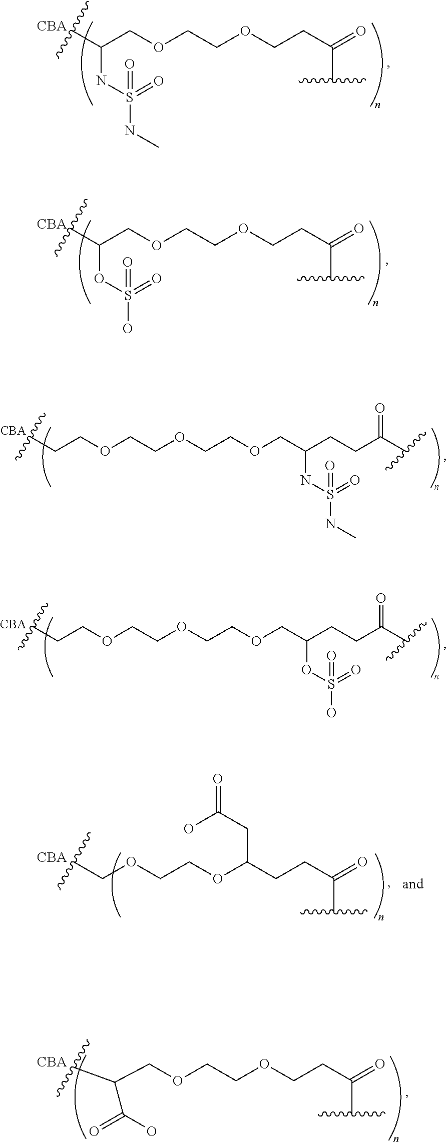

wherein n is 1-8, including all integer values and range there between (e.g., 1, 2, 3, 4, 5, 6, 7, or 8);
   and wherein the wavy line with ‘CBA’ indicates the connection of the linker to the portion of the glycoconjugate comprising the CBA, and the other wavy line indicates the connection of the linker to the remainder of the payload.
   In some embodiments, a branched linker may be used to increase the number of drug moeities attached, so as to achieve a higher DAR.

Unconjugated PBD Drug-Linkers

As discussed herein, the glycoconjugates described herein may be synthesised by conjugating the glycosylated cell-binding agents described herein with suitable unconjugated payloads. Thus, the glycoconjugates comprising the conjugated PBD drug-linkers described above in the section entitled “Conjugated PBD drug-linkers” may be synthesised by conjugating the glycosylated cell-binding agents described herein with the unconjugated PBD-drug-linkers described below.

In some embodiments the unconjugated PBD drug-linker payload has a formula selected from the group consisting of:

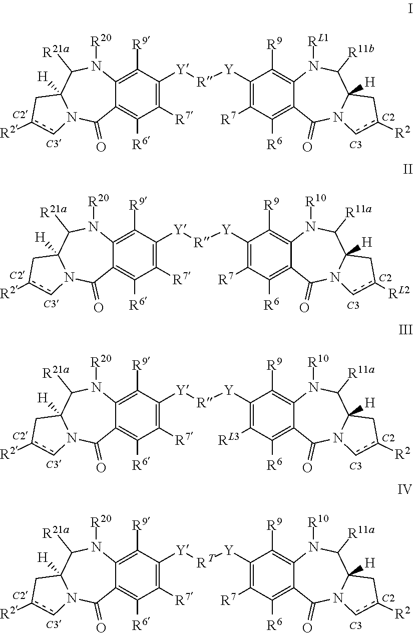

wherein:
all the substituents are defined as set out above in the section entitled “Conjugated PBD drug-linkers”; apart from,
the substituent G^{LL} which is replaced with the substituent G^{L} and selected from the group consisting of:

| | |
|---|---|
| (G^{L8}) | 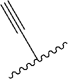 [SMILES: *C#C] |
| (G^{L9}) | 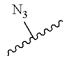 [SMILES: *N=[N+]=[N-]] |
| (G^{L10}) | 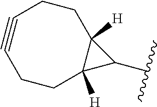 [SMILES: *C1[C@H]2CCC#CCC[C@@H]12] |
| (G^{L11}) | 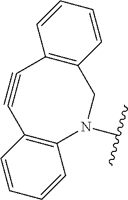 [SMILES: *N1Cc2ccccc2C#Cc2ccccc21] |
| (G^{L12}) | 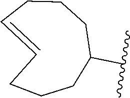 [SMILES: *C1CCC/C=C/CCC1] |
| (G^{L13}) | 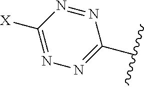 |

PBD Compounds

A PBD (pyrrolobenzodiazepine) compound is a compound comprising the following substructure:

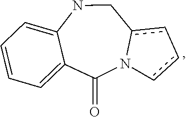

wherein any atom may be further substituted with any functional group.

In some embodiments the PBD compound is a PBD dimer. PBD dimers have been shown to form sequence selective, non-distorting and potently cytotoxic DNA interstrand cross-links in the minor groove of DNA. Typically therefore the PBD is able to bind to, and form interstrand cross-links in the minor groove of target cell DNA.

General

PBD compounds of use in the present disclosure may be, comprise, or release upon metabolism a compound of formula I:

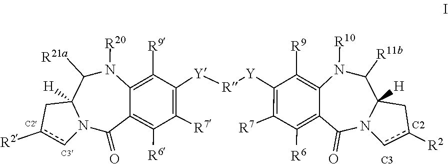

wherein:
[C6]
R⁶ is selected from H, R, OH, OR, SH, SR, NH₂, NHR, NRR′, nitro, Me₃Sn and halo;
R^{6′} is selected from H, R, OH, OR, SH, SR, NH₂, NHR, NRR′, nitro, Me₃Sn and halo;
[C9]
R⁹ is selected from H, R, OH, OR, SH, SR, NH₂, NHR, NRR′, nitro, Me₃Sn and halo;
R^{9′} is selected from H, R, OH, OR, SH, SR, NH₂, NHR, NRR′, nitro, Me₃Sn and halo;
[Y]
Y is selected from O, S, or NH;
Y′ is selected from O, S, or NH;
[C2]
when there is a double bond present between C2 and C3, R² is selected from the group consisting of:
(ia) C₅₋₁₀ aryl group, optionally substituted by one or more substituents selected from the group comprising: halo, nitro, cyano, hydroxy, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

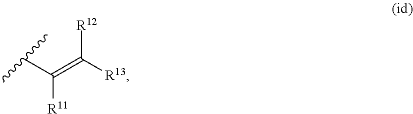

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

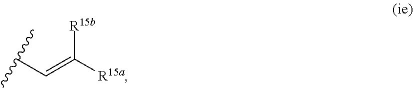

wherein one of R^{15a} and R^{15b} is H and the other is selected from: phenyl, which phenyl is optionally substituted by a group selected from halo, methyl, methoxy; pyridyl; and thiophenyl; and

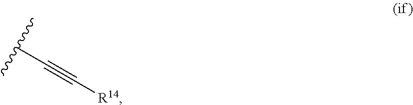

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is selected from H, OH, F, diF and

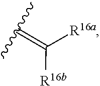

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
[C2′]
when there is a double bond present between C2′ and C3′, R^{2′} is selected from the group consisting of:
(iia) C₅₋₁₀ aryl group, optionally substituted by one or more substituents selected from the group comprising: halo, nitro, cyano, hydroxy, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl;

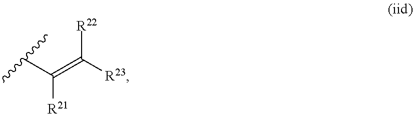

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

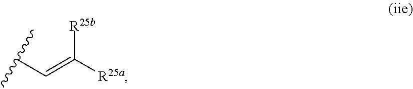

wherein one of R^{25a} and R^{25b} is H and the other is selected from: phenyl, which phenyl is optionally substituted by a group selected from halo, methyl, methoxy; pyridyl; and thiophenyl; and

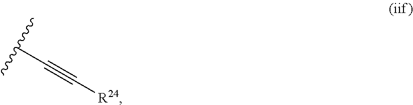

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R^{2′} is selected from H, OH, F, diF and

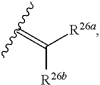

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
[C9]
R⁷ is selected from H, R, OH, OR, SH, SR, NH₂, NHR, NHRR′, nitro, Me₃Sn and halo;
R^{7′} is selected from H, R, OH, OR, SH, SR, NH₂, NHR, NHRR′, nitro, Me₃Sn and halo;
[R″]
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
[N10-C11]
(a) R¹⁰ is H, and R^{11a} is OH or OR^{A}, where R^{A} is C₁₋₄ alkyl; or
(b) R¹⁰ and R^{11a} form a nitrogen-carbon double bond between the nitrogen and carbon atoms to which they are bound; or
(c) R¹⁰ is H and R^{11a} is SO_{z}M, where z is 2 or 3 and M is a monovalent pharmaceutically acceptable cation; or
(d) R¹⁰ is H and R^{11a} is H; or
(e) R^{11a} is OH or OR^{A}, where R^{A} is C₁₋₄ alkyl and R¹⁰ is selected from:

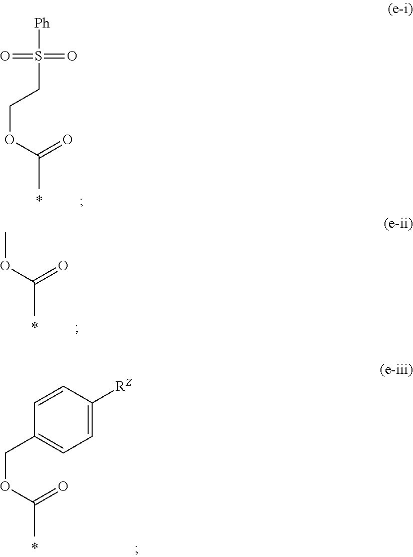

where R^{Z} is selected from:

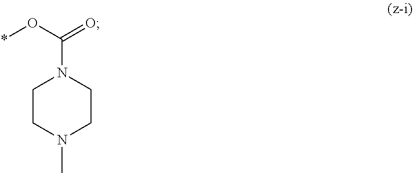 [SMILES: *OC(=O)N1CCN(C)CC1]

(z-ii) OC(═O)CH₃;
(z-iii) NO₂;
(z-iv) OMe;
(z-v) glucuronide;
(z-vi) —C(═O)—X₁—NHC(═O)X₂—NH—R^{ZC}, where —C(═O)—X₁—NH— and —C(═O)—X₂—NH— represent natural amino acid residues and R^{ZC} is selected from Me, OMe, OCH₂CH₂OMe;
(a) R²⁰ is H, and R^{21a} is OH or OR^{A}, where R^{A} is C₁₋₄ alkyl; or
(b) R²⁰ and R^{21a} form a nitrogen-carbon double bond between the nitrogen and carbon atoms to which they are bound; or
(c) R²⁰ is H and R^{21a} is SO_{z}M, where z is 2 or 3 and M is a monovalent pharmaceutically acceptable cation; or
(d) R²⁰ is H and R^{21a} is H; or
(e) R^{21a} is OH or OR^{A}, where R^{A} is C₁₋₄ alkyl and R²⁰ is selected from:

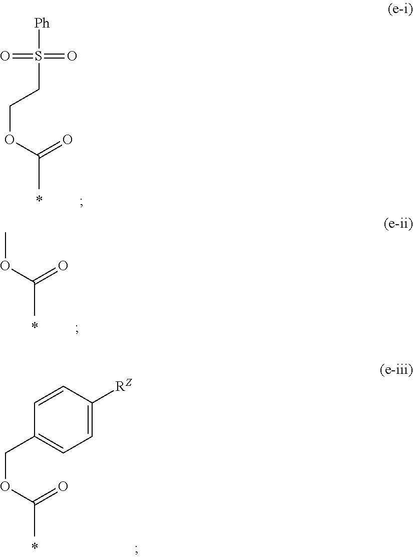

where R^{Z} is selected from:

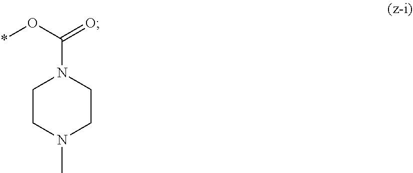 [SMILES: *OC(=O)N1CCN(C)CC1]

(z-ii) OC(═O)CH₃;
(z-iii) NO₂;
(z-iv) OMe;
(z-v) glucuronide;
(z-vi) —C(═O)—X₁—NHC(═O)X₂—NH—R^{ZC}, where —C(═O)—X₁—NH— and —C(═O)—X₂—NH— represent natural amino acid residues and R^{ZC} is selected from Me, OMe, and OCH₂CH₂OMe.

### Preferred Embodiments

In some embodiments PBD-compound is, comprises, or releases upon metabolism a compound selected from the group consisting of:

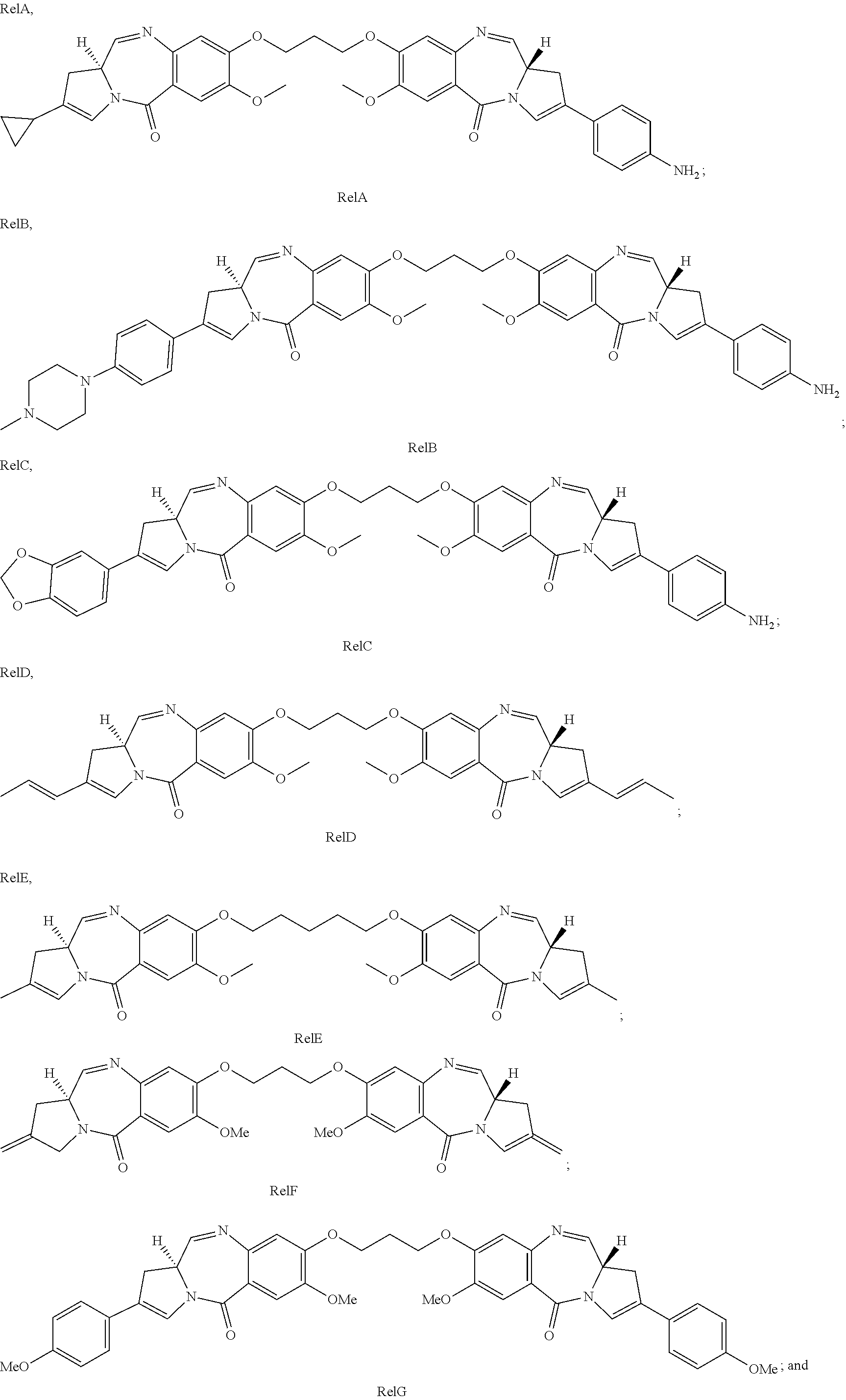

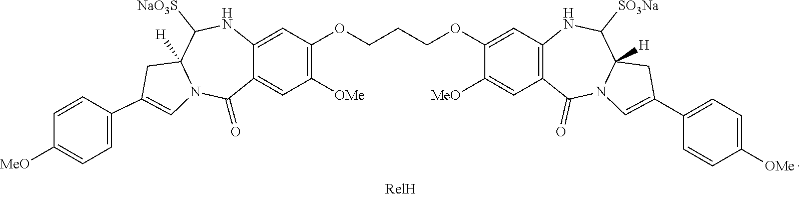

The PBD-compound RelE is particularly preferred.

Alternative Definition of Linkers

Linkers bind the payload with the remainder of the sialic acid derivative, and the conjugated payload may also be depicted as follows:

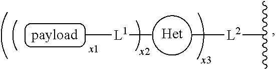

wherein payload is a PBD compound, Het represents a heterocyclic system (such as a group derived from a heterocyclic compound, which moiety has from 3 to 20 ring atoms), L¹ is selected from null (i.e. a single bond) or sublinker from Het to payload, L² is selected from null (i.e. a single bond) or sublinker from Het to the remainder of the sialic acid derivative, x1 is an integer selected from 1, 2, 3, 4, 5, 6, 7, or 8; x2 is an integer selected from 1, 2, 3, 4, 5, 6, 7, or 8; and x3 is an integer selected from 1, 2, 3, 4, 5, 6, 7, or 8. For embodiments in which one linker includes multiple payloads (i.e., at least one of x1, x2, or x3>1), the payloads can be the same or different.

For embodiments featuring multiple linker-payloads moieties attached to the same sialic acid derivative (i.e., at least two of QQ, XX, YY, and ZZ are conjugated payloads), the identities of the payloads, Het, L¹, L², x1, x2, and x3 can be the same or different.

Exemplary heterocycle systems include fused polycyclic heterocycle systems.

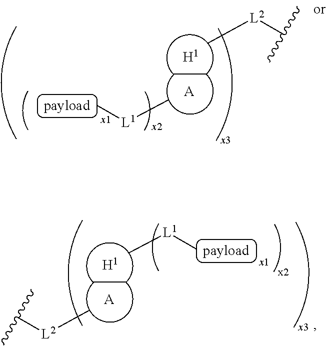

wherein H represents a heterocyclic ring, and A represents a carbocyclic or heterocyclic ring, preferably a ring having 8 atoms in the ring skeleton. Exemplary 8-atoms rings include cyclooctane, cyclooctene, aza-cyclooctane, aza-cyclooctene, 2-azacyclooctanone and unsaturated derivatives thereof. In some embodiments the 8-atom ring can be fused to one or more aromatic rings.

The heterocyclic ring represented by H¹ may be formed from cycloaddition reaction between (a) either a 1,3 dipole or 1,2,4,5 tetrazine and (b) either a strained alkyne or strained alkene. Preferred strained alkynes include cyclooctyne and preferred strained alkenes include trans-cyclooctene. Heterocyclic rings include, but are not limited to, triazoles, 1,2 pyridazines, oxazoles, isooxazoles, oxadiazoles, and saturated and partially unsaturated analogs of such rings.

In some embodiments (eg. x2 and x3=1), the heterocyclic system can have the formula:

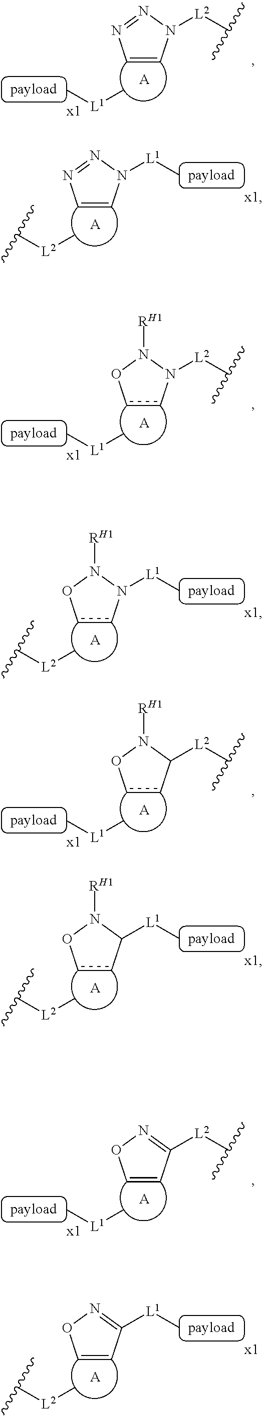

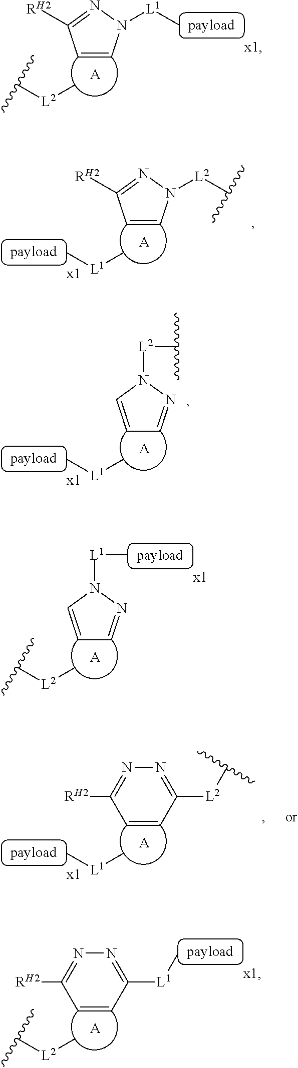

wherein x is as defined above, R^{H1} is selected from H, C₁₋₄alkyl, C₅₋₂₀aryl, C₁₋₄alkyl-C₅₋₂₀aryl, and may together with L¹ or L² form a ring;

R^{H2} is selected from H, C₁₋₄alkyl, C₅₋₂₀aryl, C₁₋₄alkyl-C₅₋₂₀aryl, and

[symbol] represents a single or double bond. In certain embodiments, the A ring can have the formula:

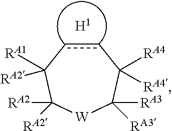

wherein R^{A1}, R^{A1′}, R^{A2}, R^{A2′}, R^{A3}, R^{A3′}, R^{A4}, and R^{A4′} are independently selected from null, H, F, Cl, Br, I, C₁₋₄alkyl, C₁₋₄alkoxy, C₅₋₂₀aryl; and wherein any one of R^{A1}, R^{A1′}, R^{A2}, R^{A2′}, R^{A3}, R^{A3′}, R^{A4} and R^{A4′} can be L¹ or L²; wherein any two or more of R^{A1}, R^{A1′}, R^{A2}, R^{A2′}, R^{A3}, R^{A3′}, R^{A4}, and R^{A4′} can together form a ring; for instance R^{A1} and R^{A2}, as well as R^{A3} and R^{A4} and can each together form an aromatic ring, while R^{A1′}, R^{A2′}, R^{A3′}, and R^{A4′} are each null;
When one of R^{A1}, R^{A1′}, R^{A2}, R^{A2′}, R^{A3}, R^{A3′}, R^{A4}, and R^{A4′} is L¹ or L², W can be CH₂CH₂. When none of R^{A1}, R^{A1′}, R^{A2}, R^{A2′}, R^{A3}, R^{A3′}, R^{A4}, and R^{A4′} are L¹ or L², W can be a group having the formula:

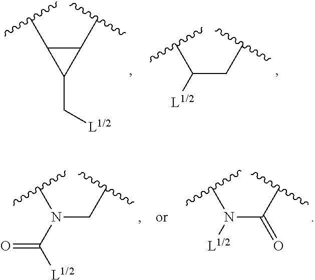

wherein L^{1/2} represents either L¹ or L²;

with the proviso that when one of W, R^{A1}, R^{A1′}, R^{A2}, R^{A2′}, R^{A3}, R^{A3′}, R^{A4}, and R^{A4′} includes L¹, none of W, R^{A1}, R^{A1′}, R^{A2}, R^{A2′}, R^{A3}, R^{A3′}, R^{A4}, and R^{A4′} includes L²; and

when one of W, R^{A1}, R^{A1′}, R^{A2}, R^{A2′}, R^{A3}, R^{A3′}, R^{A4}, and R^{A4′} includes L², none of W, R^{A1}, R^{A1′}, R^{A2}, R^{A2′}, R^{A3}, R^{A3′}, R^{A4}, and R^{A4′} includes L¹.

In some embodiments, the A ring can have the formula:

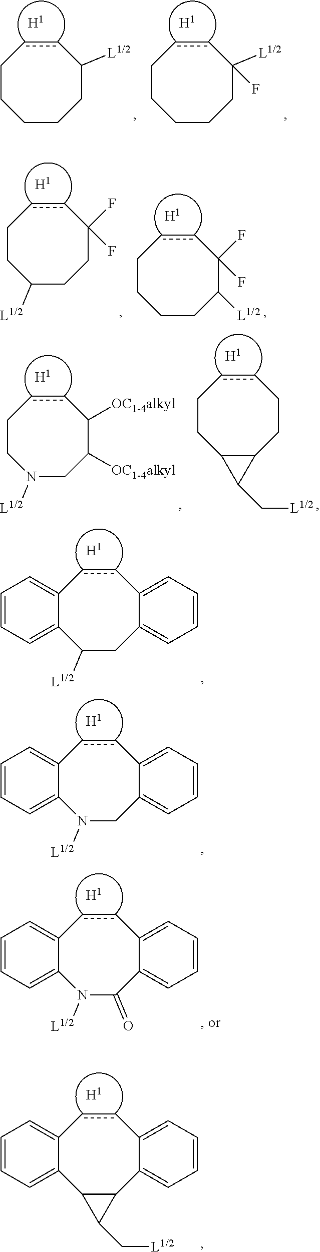

Although not depicted above, it is understood that if L¹ is connected to 8-atom ring, L² will be connected to the Heterocycle, and vice versa.

In some embodiments, L² can be null (i.e. a single bond), or a group having the formula:

-L2¹-L2²-L2³-L2⁴-L2⁵-L2⁶-,

wherein:
L2¹ is bonded to the heterocyclic system and is selected from null, C₁₋₈alkylene, C₅₋₂₀arylene, C₃₋₂₀heterocyclyl, poly(ethylene), poly(acetal); poly(glycerol), S, O, NR⁴¹, OC(═O), OC(═O)NR⁴¹, NR⁴¹C(═O), NR⁴¹C(═O)O, NR⁴¹C(═O), NR⁴¹C(═O)NR⁴¹, OC(═O)O, wherein R⁴¹ is in each case selected from H and C₁₋₄alkyl;
L2² is selected from null, C₁₋₈alkylene, C₅₋₂₀arylene, C₃₋₂₀heterocyclyl, poly(ethylene), poly(acetal); poly(glycerol), S, O, NR⁴², OC(═O), OC(═O)NR⁴², NR⁴²C(═O), NR⁴²C(═O), NR⁴²C(═O)O, NR⁴²C(═O)NR⁴², OC(═O)O, wherein R⁴² is in each case selected from H and C₁₋₄alkyl;
L2³ is selected from null, C₁₋₈alkylene, C₅₋₂₀arylene, C₃₋₂₀heterocyclyl, S, poly(ethylene), poly(acetal); poly(glycerol), O, NR⁴³, OC(═O), OC(═O)NR⁴³, NR⁴³C(═O), NR⁴³C(═O), NR⁴³C(═O)O, NR⁴³C(═O)NR⁴³, OC(═O)O, wherein R⁴³ is in each case selected from H and C₁₋₄alkyl;
L2⁴ is selected from null, C₁₋₈alkylene, C₅₋₂₀arylene, C₃₋₂₀heterocyclyl, poly(ethylene), poly(acetal); poly(glycerol), S, O, NR⁴⁴, OC(═O), OC(═O)NR⁴⁴, NR⁴⁴C(═O), NR⁴⁴C(═O), NR⁴⁴C(═O)O, NR⁴⁴C(═O)NR⁴⁴, OC(═O)O, wherein R⁴⁴ is in each case selected from H and C₁₋₄alkyl;
L2⁵ is selected from null, C₁₋₈alkylene, C₅₋₂₀arylene, C₃₋₂₀heterocyclyl, poly(ethylene), poly(acetal); poly(glycerol), S, O, NR⁴⁵, OC(═O), OC(═O)NR⁴⁵, NR⁴⁵C(═O), NR⁴⁵C(═O), NR⁴⁵C(═O)O, NR⁴⁵C(═O)NR⁴⁵, OC(═O)O, wherein R⁴⁵ is in each case selected from H and C₁₋₄alkyl;
L2⁶ is bonded to the sialoside and is selected from null, C₁₋₈alkylene, C₅₋₂₀arylene, C₃₋₂₀heterocyclyl, poly(ethylene), poly(acetal); poly(glycerol), S, O, NR⁴⁶, OC(═O), OC(═O)NR⁴⁶, NR⁴⁶C(═O), NR⁴⁶C(═O), NR⁴⁶C(═O)O, NR⁴⁶C(═O)NR⁴⁶, OC(═O)O, wherein R⁴⁶ is in each case selected from H and C₁₋₄alkyl.

In certain embodiments, any two or more of L2¹, L2², L2³, L2⁴, L2⁵, and L2⁶ can together form a ring.

The skilled person understands that selection of null for each of L2¹, L2², L2³, L2⁴, L2⁵, and L2⁶ produces an embodiment in which L² is null.

In certain embodiments, L2⁶ is NHC(O)NH, while in other embodiments, L2⁶ is heterocyclyl or heteroaryl, for instance a triazole, a 1,2 pyridazine, an oxazole, an isoxazole, an oxadiazole, and saturated and partially unsaturated analogs thereof.

In certain embodiments, L2¹ is arylene, for instance 1,4-phenylene.

In some embodiments, L2¹ is null, OC(O)NH, C₁₋₈alkylene, preferably C₁₋₃alkylene or arylene, for instance 1,4-phenylene, L2² is null or C₁₋₈alkylene, preferably C₁₋₃alkylene, L2³ is null, C(═O)NH, NHC(═O), NHC(═O)O, or OC(═O)NH; L2⁴ is null or poly(ethylene), L2⁵ is null or C₁₋₈alkylene, preferably C₁₋₃alkylene, and L2⁶ is null or heterocyclyl or heteroaryl, for instance a triazole, a 1,2 pyridazine, an oxazole, an isoxazole, an oxadiazole, and saturated and partially unsaturated analogs thereof.

In certain embodiments, L2¹ is OC(O)NH, and each of L2², L2³, L2⁴, L2⁵, and L2⁶ is null.

By way of example, certain selections for x, L2¹, L2², L2³, L2⁴, L2⁵, and L2⁶ will produce embodiments having the following partial structures:

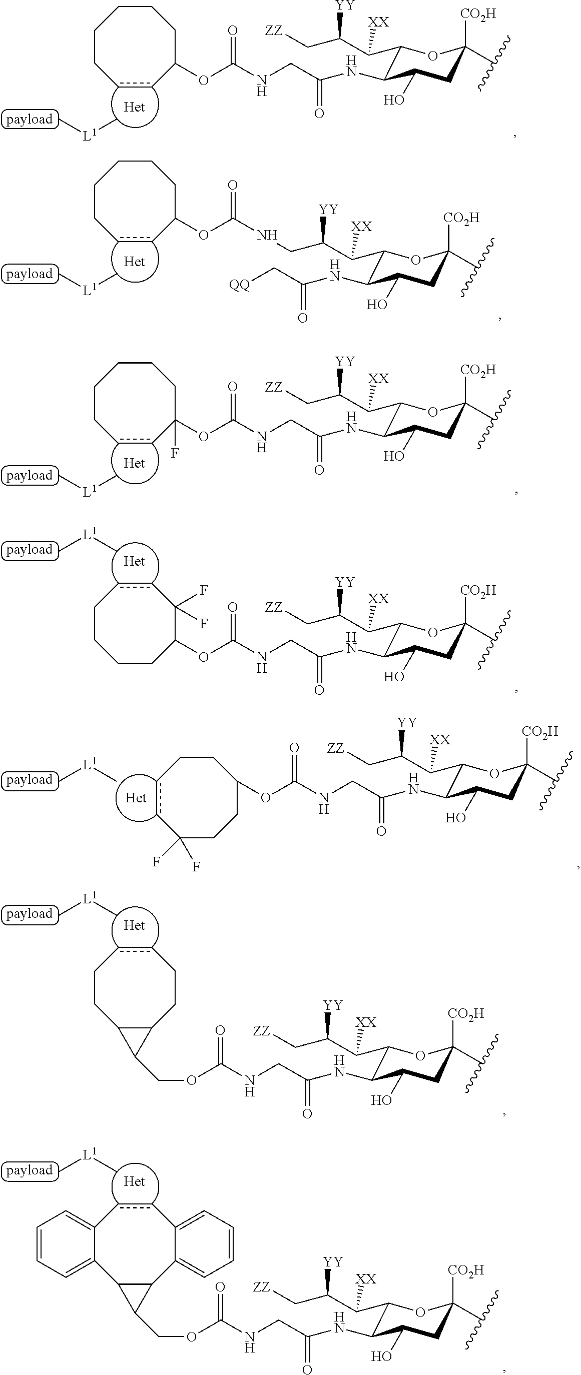

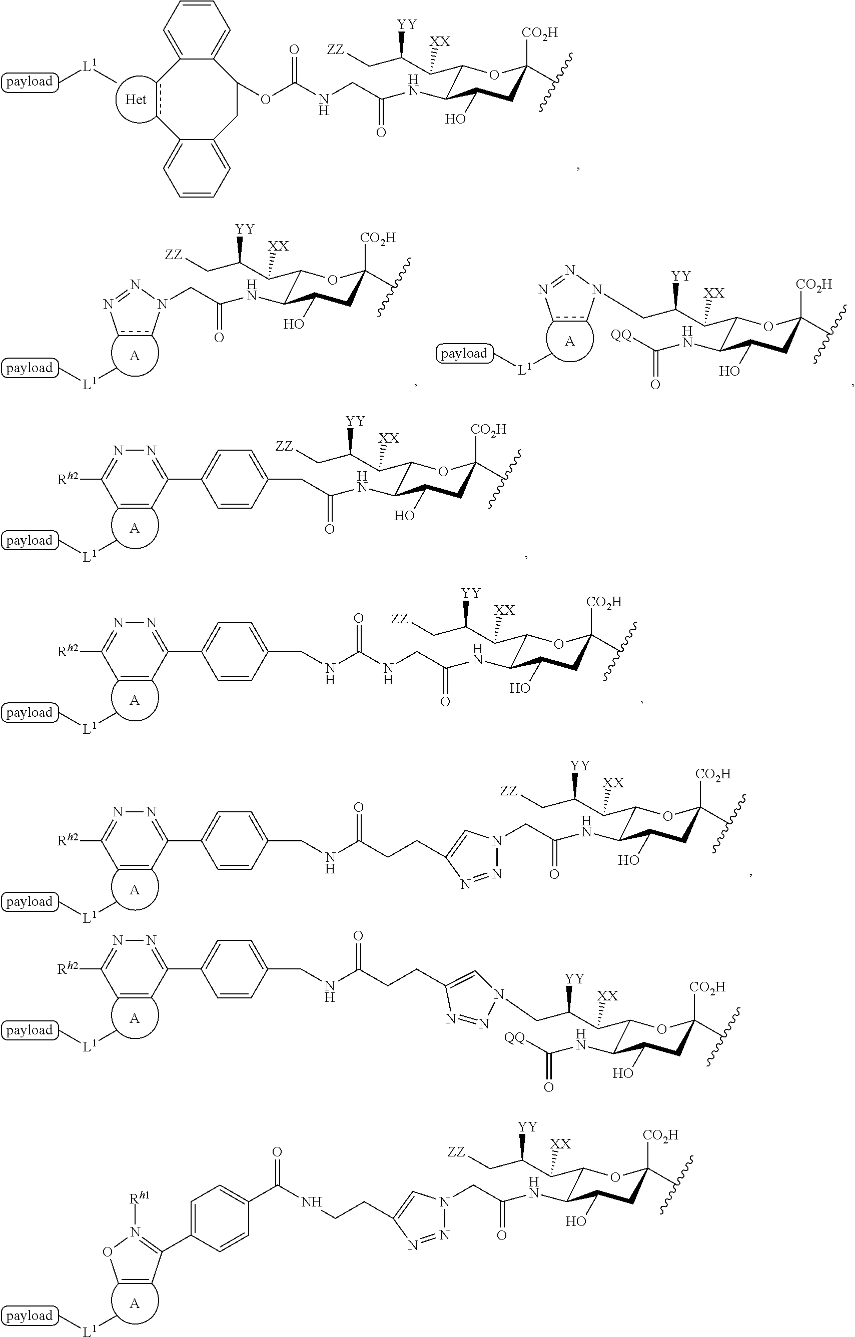

wherein L¹, payload, R^{h2}, A, QQ, ZZ, YY, and XX are as defined above. As aforementioned, more than one of QQ, ZZ, YY, and XX can be a conjugated payload, having the same of different payload, and the same or different linker.

In some embodiments, L¹ can be null, or a group having the formula:

-L1¹-L1²-L1³-L1⁴-L1⁵-L1⁶-,

wherein:
L1¹ is bonded to the heterocyclic system and is selected from null, C₁₋₈alkylene, C₅₋₂₀arylene, C₃₋₂₀heterocyclyl, poly(ethylene), poly(acetal); poly(glycerol), S, O, NR³¹, OC(═O), OC(═O)NR³¹, NR³¹C(═O), NR³¹C(═O), NR³¹C(═O)NR³¹, OC(═O)O, wherein R³¹ is in each case selected from H and C₁₋₄alkyl;
L1² is selected from null, C₁₋₈alkylene, C₅₋₂₀arylene, C₃₋₂₀heterocyclyl, poly(ethylene), poly(acetal); poly(glycerol), S, O, NR³², OC(═O), OC(═O)NR³², NR³²C(═O), NR³²C(═O), NR³²C(═O)NR³², OC(═O)O, wherein R³² is in each case selected from H and C₁₋₄alkyl;
L1³ is selected from null, C₁₋₈alkylene, C₅₋₂₀arylene, C₃₋₂₀heterocyclyl, poly(ethylene), poly(acetal); poly(glycerol), S, O, NR³³, OC(═O), OC(═O)NR³³, NR³³C(═O), NR³³C(═O), NR³³C(═O)NR³³, OC(═O)O, wherein R³³ is in each case selected from H and C₁₋₄alkyl;
L1⁴ is selected from null, C₁₋₈alkylene, C₅₋₂₀arylene, C₃₋₂₀heterocyclyl, poly(ethylene), poly(acetal); poly(glycerol), S, O, NR³⁴, OC(═O), OC(═O)NR³⁴, NR³⁴C(═O), NR³⁴C(═O), NR³⁴C(═O)NR³⁴, OC(═O)O, wherein R³⁴ is in each case selected from H and C₁₋₄alkyl;
L1⁵ is selected from null, C₁₋₈alkylene, C₅₋₂₀arylene, C₃₋₂₀heterocyclyl, poly(ethylene), poly(acetal); poly(glycerol), S, O, NR³⁵, OC(═O), OC(═O)NR³⁵, NR³⁵C(═O), NR³⁵C(═O), NR³⁵C(═O)NR³⁵, OC(═O)O, wherein R³⁵ is in each case selected from H and C₁₋₄alkyl;
L1⁶ is bonded to the payload and is selected from null, C₁₋₈alkylene, C₅₋₂₀arylene, C₃₋₂₀heterocyclyl, poly(ethylene), poly(acetal); poly(glycerol), S, O, NR³⁶, OC(═O), OC(═O)NR³⁶, NR³⁶C(═O), NR³⁶C(═O), NR³⁶C(═O)NR³⁶, OC(═O)O, wherein R³⁶ is in each case selected from H and C₁₋₄alkyl.

In certain embodiments, any two or more of L1¹, L1², L1³, L1⁴, L1⁵, and L1⁶ can together form a ring.

L1³ can be a branched C₁₋₈alkylene, arylene, heteroaryl, or heterocyclyl group. By way of example, L1³ can be a phenyl group having the formula:

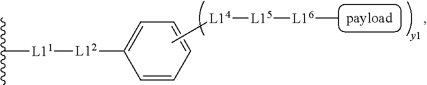

wherein y1 is any substitution number permitted by valence. In the exemplary formula above, x1 can be 1, 2, 3, 4, or 5. The skilled person recognizes that other possible L1³ groups will give rise to different x1 possibilities. In other instances, L1³ can be a branched alkylene, e.g., a methylene having the formula:

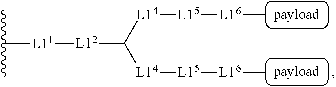

or a methine having the formula:

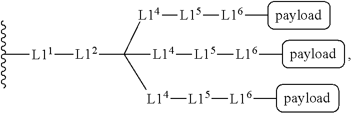

In some embodiments, L1³ can include a polymeric group, for instance a poly(glycerol) having the formula:

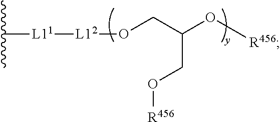

or

a polyacetal having the formula:

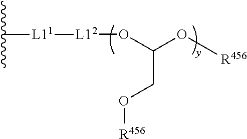

wherein y is from 1-1,000; and

R⁴⁵⁶ is selected from hydrogen or a moiety of Formula (456):

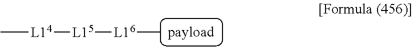

and the number of times that R⁴⁵⁶ is the moiety of Formula 456 is less than 30.

A cleavable L¹ group will include at least one functional group that undergoes bond-breaking under environmental conditions. Cleavable groups include acid-sensitive groups, redox sensitive groups, and enzyme-cleavable groups, for instance, protease cleavable groups. Exemplary acid-sensitive groups include Schiff bases/imines, hydrazones, boronic esters, and acetals. Exemplary redox-sensitive groups include thioacetals, oxalate esters, disulfides, peptides, and diselenide groups. Exemplary enzyme cleavable groups include peptide fragments Val-Lys, Val-Ala, Val-Arg, Phe-Lys, and Val-Cit.

In some instances, L¹ can include a self-immolative spacer. A self-immolative spacer refers to a chemical moiety bonded to a selectively cleavable group, wherein activation of the cleavable group results in a cascade of reactions that ultimately liberates the payload from the spacer. Exemplary self-immolative spacers include p-aminobenzyl alcohols, p-hydroxybenzyl alcohols, 2-aminoimidazol-5-methanol moieties, ortho- or para-aminobenzylacetals, aminobutyric acid amides, 1,2 diamino ethylene, 1,3 diaminopropylene

In some embodiments, L¹ can include a self-immolative spacer, cleavable group, and optional additional linker, e.g., a conjugate having the formula:

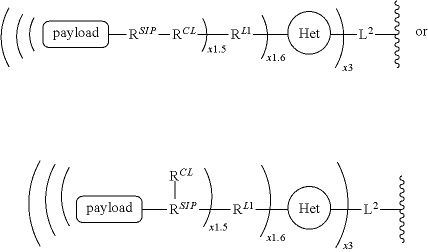

wherein R^{SIP} is one or more self-immolative spacers, R^{CL} is a cleavable group, and R^{L1}, when present, is an additional linker, x1.5 is an integer selected from 1, 2, 3, 4, 5, 6, 7, and 8; and x1.6 is an integer selected from 1, 2, 3, 4, 5, 6, 7, and 8.

The payload can be bonded to L¹ or R^{SIP} via a carbamate group, e.g.,

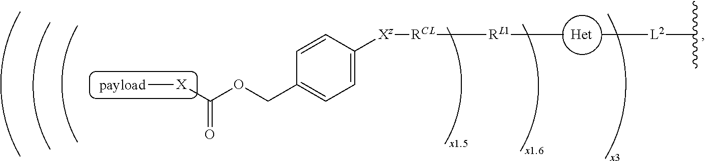

wherein X is a nitrogen atom in the payload, and X^{z} is O, NH, or NC₁₋₄alkyl. Benzyl self-immolating spacers depicted above may be further substituted one or more times by electron withdrawing groups like nitro, fluoro, trifluoromethyl, and the like. R^{ea1} and R^{ea2} can be independently selected from H, C₁₋₄alkyl, or (CH₂CH₂O)ₙCH₂CH₂OH, wherein n is from 0, 1, 2, or 3. The carbamate linkage is appropriate for linking to the nitrogen of the imine in a PBD moiety

In some cases R^{CL} is a peptidyl residue, e.g.,

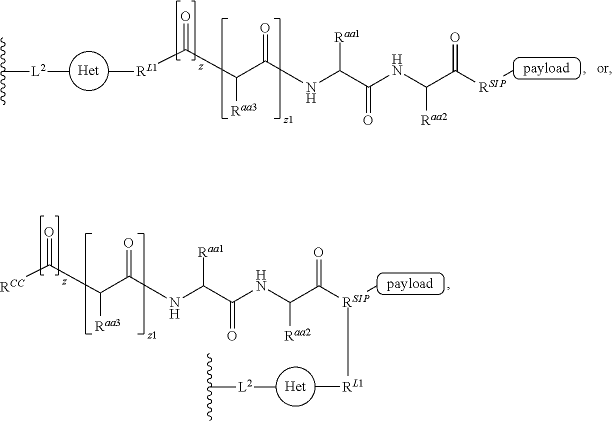

wherein z is 1 or 0, z1 is 1 or 0, R^{CC} is H, peptidyl, C₁₋₆alkyl, C₃₋₆cycloalkyl, C₅₋₂₀aryl or C₃₋₂₀heterocyclyl, R^{aa1}, R^{aa2}, and R^{aa3} are independently selected from H, C₁₋₆alkyl optionally substituted with phenyl, COOH, NH₂, COHNH₂, NHC(O)NH₂. In certain embodiments z1 is 0 and R^{aa1} is isopropyl and R^{aa2} is (—CH₂)₄NH₂, (—CH₂)₃NHC(O)NH₂, (—CH₂)₃NHC(NH)NH₂, or CH₃. In other cases z1 is 0 and R^{aa1} is benzyl and R^{aa2} is (—CH₂)₄NH₂. In yet further embodiments, z1 is 1, R^{aa1} is isopropyl, R^{aa2} is (—CH₂)₃NHC(O)NH₂, and R^{aa3} is (—CH₂)COOH.

The peptidyl residues may have the following formula:

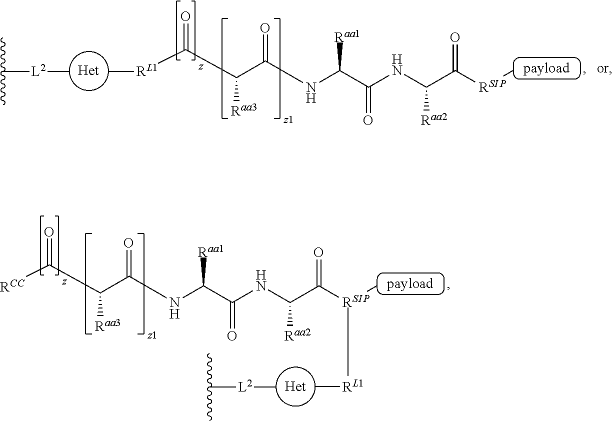

wherein R^{cc}, z, z1, R^{aa1}, R^{aa2}, R^{aa3}, R^{SIP} are as defined above.

In some instances, R^{SIP} can be a 4-aminobenzyl alcohol having the formula, exemplified below when z1 is 0:

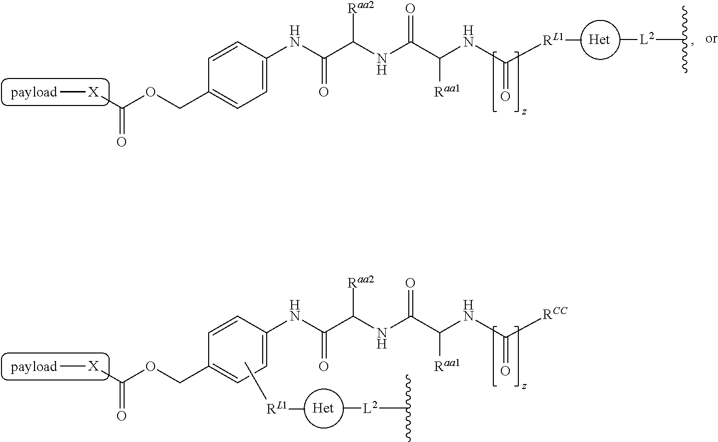

R^{SIP} can be a 4-aminobenzyl alcohol when z1 is 1.

In some instances, R^{CL} is a peptide group having the formula:

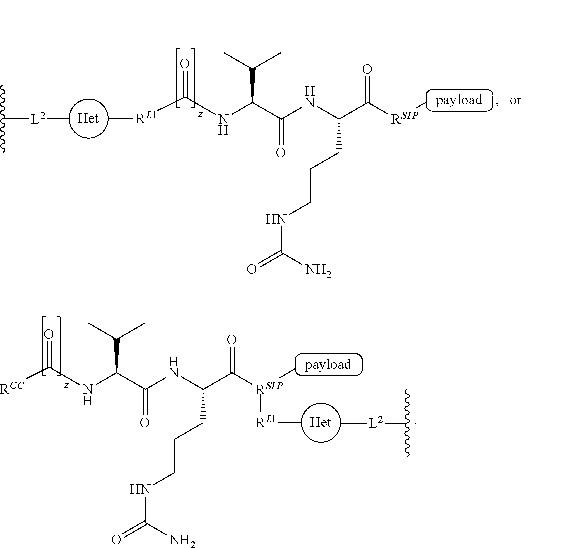

In other embodiments R^{CL} can be a gluconic acid residue, for instance:

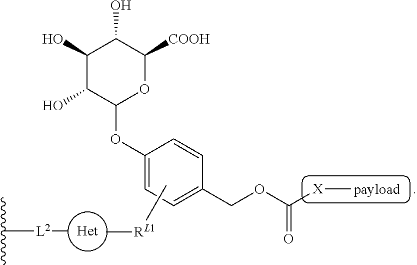

In other embodiments, the cleavable group can be a disulfide:

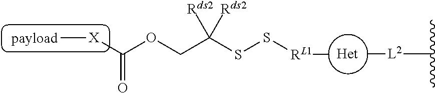

wherein R^{ds1} and R^{ds2} are independently selected from H and C₁₋₄alkyl. In some instances, R^{ds1} and R^{ds2} are both hydrogen, or R^{ds1} and R^{ds2} are both methyl. In other instances R^{ds1} is hydrogen and R^{ds2} is C₁₋₄aklyl.

Suitable R^{L1} groups include alkyl chains (CH₂)ₙ where n is from 2-20, 2-10, 5-10, 5-15, 10-15, or 10-20, aryl rings, cycloalkyl rings (especially cyclohexyl), polyethylene glycols (CH₂CH₂O)ₘ wherein m is from 1-30, 5-30, 10-30, 15-30, 1-15, 2-10, 5-10 or 5-15. In some embodiments, R^{L1} can be a combination of two or more of the enumerated groups.

Methods of Manufacture

Manufacture of Glycoconjugates

In a third aspect the present disclosure provides a method for the preparation of the glycoconjugates described herein, the method comprising the steps of:
(i) providing a Sd(A^{F})ₓ acceptor having the formula:

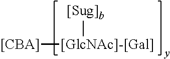

wherein CBA, GlcNAc, Sug, Gal, b, and y are defined as described elsewhere herein for glycoconjugates; and
(ii) contacting the Sd(A^{F})ₓ acceptor with a compound of the formula Sd(A^{F})ₓ-P* in the presence of a glycosyltransferase to yeild a glycosylated cell-binding agent, wherein:
   Sd(A^{F})ₓ is as defined as described elsewhere herein except that “A^{F}” represents a functional group A^{F} instead of a conjugated payload A^{P}; and P* is a nucleoside phosphate moiety; and
(iii) reacting the glycosylated cell-binding agent of (ii) with a compound of the formula payload-G^{L}, wherein the payload is as above and G^{L} is linker group reactive with the functional group A^{F} of the glycosylated cell-binding agent

In some embodiments, the Sd(A^{F})ₓ acceptor has the formula:

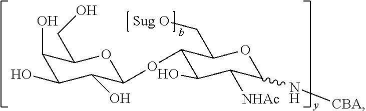

wherein Sug, b, y, and CBA are as defined above,

In some embodiments, the saccharide moiety is connected to the CBA through a α-N-glycosidic bond to give a Sd(A^{F})ₓ acceptor having the formula:

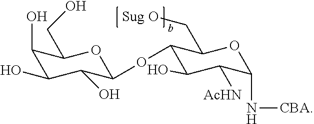

In embodiments where the cell-binding agent is a peptide or polypeptide (such as an antibody), or comprises a peptide or polypeptide portion, the saccharide moiety may be conjugated to the antibody through an asparagine side chain via an α-N-glycosidic bond to give a Sd(A^{F})ₓ acceptor having the formula:

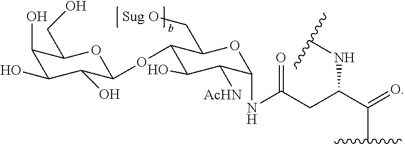

In preferred embodiments the CBA is an antibody. In some cases the GlcNAc moiety is conjugated to the antibody at the asparagine 297 (Asn297) residue according to the EU index as set forth in Kabat. In embodiments wherein y is 1, the GlcNAc moiety may be conjugated to one of the Asn297 residues in the Fc domain. In embodiments wherein y is 2, a GlcNAc moiety is conjugated to each of the two Asn297 residues in the Fc domain. In embodiments where the antibody has been modified—for example by chain elongation or truncation—the GlcNAc moiety may be conjugated to the asparagine residue corresponding to Asn297 of the unmodified antibody.

In some embodiments, the compound of the formula Sd(A^{F})ₓ-P* has the formula:

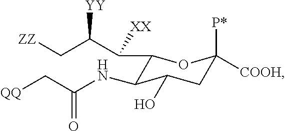

wherein:
P* is a nucleoside phosphate moiety; and
QQ is hydrogen or a functional group A^{F};
ZZ is hydroxyl or a functional group A^{F};
YY is hydroxyl or a functional group A^{F}; and/or
XX is hydroxyl or a functional group A^{F};

and wherein at least one of QQ, ZZ, YY, and XX is a functional group A^{F}.

In preferred embodiments, G^{L} is as defined in the section herein titled ‘Unconjugated PBD drug-linkers’. In most referred embodiments, the compound “payload-G^{L}” is an unconjugated PBD drug-linker payload as defined in the section herein titled ‘Unconjugated PBD drug-linkers’.

The Sd(A^{F})ₓ acceptor above, maybe provided by a number of different methods, such as de novo glycosylation of a previously unglcycosylated site on a CBA, or modification of extant CBA glycans. In the either of the de novo or extant routes, glycosylation and modification may be performed via chemical synthesis, enzymatic processing, or a mixture of the two.

Typically, the Sd(A^{F})ₓ acceptor above, is provided by modifying extant CBA glycans. The process of modifying extant glycans is often termed ‘glycan remodelling’ or simply ‘remodelling’. In preferred embodiments remodelling is performed mostly, if not exclusively, with enzymes, since these catalysts are characterized by high specificity, high activity, and suitability for use under the conditions suitable for CBA biomolecule stability.

Accordingly, in some embodiments the Sd(A^{F})ₓ acceptor above, is provided by a method comprising the steps of:
a) providing a Gal acceptor having the formula:

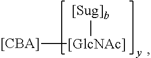

wherein CBA, GlcNAc, Sug, b, and y are defined as described elsewhere herein for glycoconjugates; and
b) contacting the Gal acceptor with a compound of the formula Gal-P* in the presence of a galactosyltransferase, wherein Gal and P* are as defined above;
   optionally wherein,
c) the Gal acceptor is produced by contacting with a glycosidase a oligoglycosylated cell-binding agent having the formula:

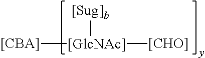

wherein CHO is a carbohydrate moiety.

In some embodiments the Gal acceptor has the formula:

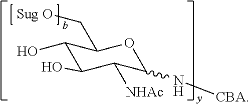

In some embodiments the Gal acceptor is connected to the CBA through an α-N-glycosidic bond with the formula:

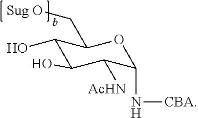

In embodiments where the cell-binding agent is a peptide or polypeptide (such as an antibody), or comprises a peptide or polypeptide portion, the GlcNAc moiety may be conjugated to the antibody through an asparagine side chain via an α-N-glycosidic bond with the formula:

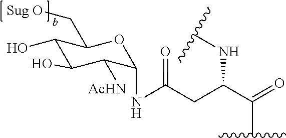

In preferred embodiments the CBA is an antibody. In some cases the GlcNAc moiety is conjugated to the antibody at the asparagine 297 (Asn297) residue according to the EU index as set forth in Kabat. In embodiments wherein y is 1, the GlcNAc moiety may be conjugated to one of the Asn297 residues in the Fc domain. In embodiments wherein y is 2, a GlcNAc moiety is conjugated to each of the two Asn297 residues in the Fc domain. In embodiments where the antibody has been modified—for example by chain elongation or truncation—the GlcNAc moiety may be conjugated to the asparagine residue corresponding to Asn297 of the unmodified antibody.

In some embodiments where the CBA is an antibody, the antibody is remodeled such that a GlcNAc moiety (with or without the C6 Sug) is linked to Asn297 (either on one or both heavy chains), and no other glycan structures are present on the antibody.

The contacting the Sd(A^{F})ₓ acceptor with a compound of the formula Sd(A^{F})x-P* in the presence of a glycosyltransferase described in part (ii), above, will typically transfer Sd(A^{F})ₓ onto terminal Galactose residues with high efficiency. Accordingly, in preferred embodiments the number of terminal galactose residues available on the Sd(A^{F})ₓ acceptor is tightly controlled so as to control the number and location of Sd(A^{F})ₓ moieties that are transferred onto the glycosylated cell-binding agent. Control of the number and location of Sd(A^{F})ₓ moieties is important for controlling the number and location of payload moieties that are conjugated to the glycosylated cell-binding agent in the subsequent payload-conjugation reaction.

Accordingly, in some embodiments the Sd(A^{F})ₓ acceptor has only 1 terminal galactose moiety. That is, y=1 and the CBA has no other terminal galactose residues that are accessible to (ie. can react with) the glycosyltransferase catalyst used in step (ii).

In other embodiments the Sd(A^{F})ₓ acceptor has only 2 terminal galactose moieties. That is, y=2 and the CBA has no other terminal galactose residues that are accessible to (ie. can react with) the glycosyltransferase catalyst used in step (ii). Included in these embodiments is a set of preferred embodiments where the CBA is an antibody (such as IgG, in particular IgG1) that has one N-glycan on each of the two heavy-chain constant regions (typically conjugated to the asparagine-297 residue according to the EU index as set forth in Kabat).

In other embodiments the Sd(A^{F})ₓ acceptor has only 3 terminal galactose moieties. That is, y=3 and the CBA has no other terminal galactose residues that are accessible to (ie. can react with) the glycosyltransferase catalyst used in step (ii).

In other embodiments the Sd(A^{F})ₓ acceptor has only 4 terminal galactose moieties. That is, y=4 and the CBA has no other terminal galactose residues that are accessible to (ie. can react with) the glycosyltransferase catalyst used in step (ii).

One-Pot Glycoconjugate Manufacture

As described above, the manufacture of the glyconjugates described herein from an oligoglycosylated cell-binding agent (such as an antibody) typically involves a method comprising the following series of steps:
a) providing an oligoglycosylated cell-binding agent having the formula:

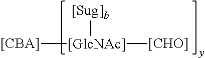

wherein CBA is a Cell Binding Agent;
GlcNAc is a N-acetyl-glucosamine moiety;
Sug is a sugar moiety, wherein b=1 or 0;
y=1 to 20; and
CHO is a carbohydrate moiety;
b) contacting the oligoglycosylated cell-binding agent with a glycosidase to produce a Gal acceptor having the formula:

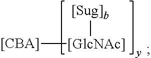

c) contacting the Gal acceptor with a compound of the formula Gal-P* in the presence of a galactosyltransferase to produce a Sd(A^{F})ₓ acceptor having the formula:

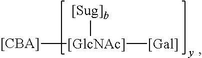

wherein Gal is a galactose moiety, and
P* is a nucleoside phosphate moiety;
d) contacting the Sd(A^{F})ₓ acceptor with a compound of the formula Sd(A^{F})ₓ-P* in the presence of a glycosyltransferase to produce a glycosylated cell-binding agent having the formula:

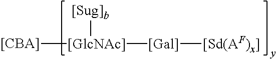

wherein:
   Sd(A^{F})ₓ is a sugar derivative comprising x functional groups A^{F}, wherein x=1, 2, 3, or 4;
e) reacting the glycosylated cell-binding agent with a compound of the formula payload-G^{L},
   wherein the payload is as defined in the above section entitled “Payload” and G^{L} is a linker group reactive with the functional group A^{F} of the glycosylated cell-binding agent,
to produce a glycoconjugate having the formula:

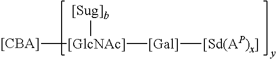

wherein:
   Sd(A^{P})ₓ is a sugar derivative comprising x conjugated payloads A^{P}, wherein x=1, 2, 3, or 4.

The present authors conducted a series of investigations with a view to maximising the yield of glycoconjugate obtainable through the above method. This research resulted in identification of reaction conditions that allowed all of the enzyme remodelling steps (ie. steps (b) to (d) in the above scheme) to be carried out at high efficiency in a single reaction vessel without the need for purification between steps, so avoiding the inevitable loss of useful intermediate that would otherwise occur during purification.

Accordingly, the present disclosure provides method for the preparation of the glycoconjugates described herein, the method comprising the steps of: (a) to (e) above, wherein steps (b), (c), and (d) are performed in the same reaction volume. In some embodiments the Gal acceptor product of step (b) is not purified from the reaction volume before it is contacted with the galactosyltransferase of step (c). In some embodiments the Sd(A^{F})ₓ acceptor product of step (c) is not purified from the reaction volume before it is contacted with the glycosyltransferase of step (d).

In some embodiments all of the steps (b), (c), and (d) are performed at the same time. In some embodiments all of steps (b), (c), and (d) are performed in parallel. In some embodiments all of steps (b), (c), and (d) are performed in sequence.

In some embodiments step (b) comprises at least a 6 hour incubation, for example at least a at least a 12 hour incubation, 24 hour incubation, at least a 36 hour incubation, or at least a 48 hour incubation. In some embodiments step (b) comprises an incubation of between 24 and 48 hours, such as about 36 hours. In some embodiments step (c) comprises at least a 6 hour incubation, for example at least a at least a 12 hour incubation, 24 hour incubation, at least a 36 hour incubation, or at least a 48 hour incubation. In some embodiments step (c) comprises an incubation of between 24 and 48 hours, such as about 36 hours. In some embodiments step (d) comprises at least a 6 hour incubation, for example at least a at least a 12 hour incubation, 24 hour incubation, at least a 36 hour incubation, or at least a 48 hour incubation. In some embodiments step (d) comprises an incubation of between 24 and 48 hours, such as about 36 hours.

In some embodiments, the incubations are at at least 15° C., such as at least 20° C., at least 25° C., at least 30° C., or least 35° C. Preferably the incubations are at no more than 50° C., such as no more than 45° C., or no more than 40° C. In some embodiments the incubations are between 30 and 45° C., such as about 37° C.

In some embodiments the method further comprises the additional step (d′) between steps (d) and (e), wherein step (d′) comprises the addition of further Sd(A^{F})ₓ-P* and/or glycosyltransferase. In some embodiments the further Sd(A^{F})ₓ-P* and/or glycosyltransferase are added to the products of step (d). For example, in some embodiments the further Sd(A^{F})ₓ-P* and/or glycosyltransferase are added on completion of the incubation of step (d). In some embodiments the further Sd(A^{F})ₓ-P* and/or glycosyltransferase are added at least a 6 hours, such as at least 12 hours, at least 24 hours, at least 36 hours, or at least a 48 hours after the Sd(AF)x acceptor product of step (c) is first contacted with a compound of the formula Sd(AF)x-P* in the presence of a glycosyltransferase in step (d). In some embodiments the further Sd(A^{F})ₓ-P* and/or glycosyltransferase are added between 24 and 48 hours, such as about 36 hours after the Sd(AF)x acceptor product of step (c) is first contacted with a compound of the formula Sd(AF)x-P* in the presence of a glycosyltransferase in step (d).

In some embodiments step (d′) comprises at least a 6 hour incubation, for example at least a at least a 12 hour incubation, 24 hour incubation, at least a 36 hour incubation, or at least a 48 hour incubation. In some embodiments step (d′) comprises an incubation of between 24 and 48 hours, such as about 36 hours. In some embodiments, the incubation is at at least 15° C., such as at least 20° C., at least 25° C., at least 30° C., or least 35° C. Preferably the incubation is at no more than 50° C., such as no more than 45° C., or no more than 40° C. In some embodiments the incubation is between 30 and 45° C., such as about 37° C.

Functional Groups A

In some embodiments, each of the one or more of the x functional groups A^{F} is independently selected from the group consisting of an azido group, an alkynyl group, and a keto group.

For example, in some embodiments Sd(A^{F})ₓ has one functional group A^{F} that is an azide group at position QQ, thus:

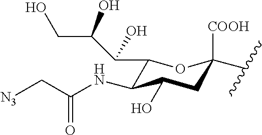

In some exemplary embodiments Sd(A^{F})ₓ has one functional group A^{F} that is an azide group at position ZZ, thus:

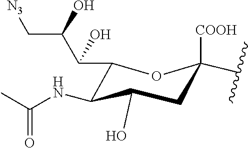

In some exemplary embodiments Sd(A^{F})ₓ has one functional group A^{F} that is an alkynyl group at position QQ, thus:

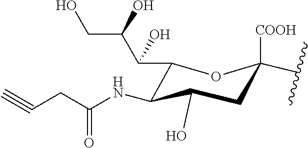

In some exemplary embodiments Sd(A^{F})ₓ has one functional group A^{F} that is an alkynyl group at position ZZ, thus:

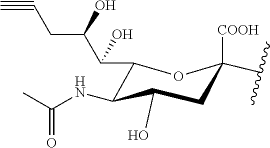

In some exemplary embodiments Sd(A^{F})ₓ has one functional group A^{F} that is a keto group at position QQ, thus:

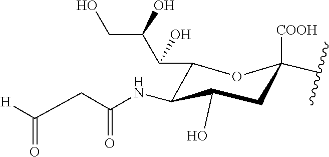

In some exemplary embodiments Sd(A^{F})ₓ has one functional group A^{F} that is a keto group at position ZZ, thus:

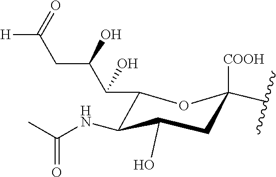

Nucleoside Phosphates

Nucleoside phosphates play roles in a range of biochemical reactions, including acting as short-term stores and transporters of chemical energy (eg. ATP and GTP), themselves being the monomer building blocks of the nucleoside polymers that encode genetic information, and—when bonded to certain classes of other compounds—forming activated intermediates in a variety of anabolic reactions.

One of the anabolic processes that involves activated intermediates comprising nucleoside phosphates is glycosylation, where conjugates of nucleoside phosphates and monosaccharides (so-called ‘nucleotide sugars’) act as glycosyl donors in glycosylation reactions catalysed by glycosyltransferase enzymes. As already noted herein, there are numerous varieties of glycosyl moieties which can be arranged in a huge array of different linkages, branching structures, and chain length. This diversity is achieved by a correspondingly broad variety of glycosyltransferases which, in turn, employ a range of different types of nucleotide sugars as glycosyl donors.

Nucleotide sugars utilised in nature typically have the formula Sd-P*, where Sd is a sugar moiety or a sugar derivative moiety as defined herein and P* is a nucleoside phosphate moiety. Typically, the nucleoside element of the nucleoside phosphate moiety is one of adenosine, guanosine, uridine, cytidine, or thymidine. The nucleoside element is then conjugated to Sd via one or more phosphate groups; typically there are two sequential phosphate groups, although the—for example—nucleotide sugar CMP-β-D-Neu5Ac comprises only a single phosphate group.

There are ten nucleotide sugars in humans which are known to act as glycosyl donors, these are:
Uridine Diphosphate based:
   UDP-α-D-Glc, UDP-α-D-Gal, UDP-α-D-GalNAc, UDP-α-D-GlcNAc, UDP-α-D-GlcA, UDP-α-D-Xyl.
Guanine Diphosphate based:
   GDP-α-D-Man, GDP-β-L-Fuc.
Cytosine Monophosphate based:
   CMP-β-D-Neu5Ac.
Cytosine Diphosphate based:
   CDP-D-Ribitol.

In addition to the range of naturally occurring sugars and sugar derivatives, it has been demonstrated that some glycosyl transferases are able to utilise nucleoside sugar substrates where the sugar has been modified with one or more non-naturally occurring groups (see, for example, WO2014/065661, and Li et al., Angew Chem Int Ed Engl., 2014, Jul. 7; 53(28):7179-82).

Accordingly, in some embodiments P* is a nucleoside phosphate moiety wherein the nucleoside element of the nucleoside phosphate moiety is one of adenosine, guanosine, uridine, cytidine, or thymidine. In some embodiments P* has two sequential phosphate groups. In some embodiments P* has a single phosphate group.

In some embodiments P* is selected from UDP, GDP, TDP, CDP, and CMP.

In some embodiments, Gal-P* as used herein refers to UDP-Gal, for example UDP-α-D-Gal.

In some embodiments P forms part of a nucleotide sugar of formula Sd-P*, wherein Sd is a sugar moiety or a sugar derivative moiety as defined herein. In some embodiments P* forms part of a nucleotide sugar of formula Sd(A^{F})ₓ-P*, wherein Sd(A^{F})ₓ is as defined herein for glycoconjugates.

In some preferred embodiments, Sd(A^{F})ₓ-P* has the formula:

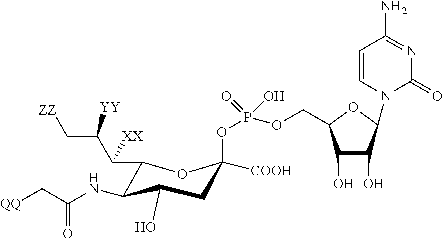

Enzymes

In preferred embodiments remodelling is performed mostly, if not exclusively, with enzymes, since these catalysts are characterized by high specificity, high activity, and suitability for use under the conditions suitable for CBA biomolecule stability.

The enzymes used in the remodelling methods described herein fall into two broad categories: (1) glycosyltransferases—enzymes that act to transfer a glycosyl moiety from a nucleotide sugar to a suitable acceptor, typically a glycan moiety present on a CBA; and (2) glycosidases—enzymes which cleave the bond between a glycosyl moiety and another moiety. Both are broad classes of enzymes, with the diversity already noted for the glycosyltransferases matched by similarly diverse and specific glycosidases.

Since enzymes are themselves chiral molecules, they typically react preferably with substrate molecules having a chirality (ie. spatial configuration) that corresponds to the chirality of the enzyme's active site. Accordingly, the saccharide molecules and moieties described herein (eg. “GlcNAc”, “Sug”, “Gal”) typically have the properties and configuration that allow for their efficient use by the enzyme catalysts. Preferably the saccharide molecules and moieties such as “GlcNAc”, “Sug”, and “Gal” described herein are ‘D’ enantiomers.

Glycosyltransferases

As noted above, in some preferred embodiments Sd(A^{F/P})x is a sialic acid derivative, wherein “sialic acid” is a generic term for N- and/or O-substituted derivatives of NeuN, such as Neu5Ac (NeuN acylated on the amine group found on C5) or Neu9Ac (NeuN acylated on the amine group found on C9). Accordingly, in those embodiments the preferred glycosyltransferase is a sialyltransferase.

The sialyltransferase may be derived from mammals, fishes, amphibians, birds, invertebrates, or bacteria. In one embodiment, the sialyltransferase is an α-(2,3)-sialyltransferase. In another embodiment, the sialyltransferase is an α-(2,6)-sialyltransferase. In yet another embodiment, the sialyltransferase is an α-(2,8)-sialyltransferase.

In a preferred embodiment, the sialyltransferase is an α-(2,6)-sialyltransferase, preferably a β-galactoside α-(2,6)-sialyltransferase 1 (ST6Gal 1). In a preferred embodiment, the sialyltransferase is a mammalian sialyltransferase. In other embodiments, the sialyltransferase rat β-galactoside α-2,6-sialyltransferase 1 (ST6Gal 1); Pasteurella multocida α-(2,3)-sialyltransferase; or CMP-N-acetylneuraminate-β-galactosamide-α-2,3-sialyltransferase (ST3Gal IV).

The glycosylation with the Sd(A^{F})x-P* may be carried out in a suitable buffer solution, such as for example phosphate, buffered saline (e.g. phosphate-buffered saline, tris-buffered saline), citrate, HEPES, tris and glycine. Suitable buffers are known in the art. Preferably, the buffer solution is phosphate-buffered saline (PBS) or tris buffer. The glycosylation is preferably performed at a temperature in the range of about 4 to about 50° C., more preferably in the range of about 10 to about 45° C., even more preferably in the range of about 20 to about 40° C., and most preferably in the range of about 30 to about 37° C. The glycosylation can be carried out at a pH in the range of about 5 to about 9, preferably in the range of about 5.5 to about 8.5, more preferably in the range of about 6 to about 8. Most preferably, the glycosylation is performed at a pH in the range of about 7 to about 8.

In some embodiments the sialyltransferase is human beta-galactoside alpha-2,6-sialyltransferase 1 (ST6Gal1). In some embodiments the sialyltransferase has the amino acid disclosed in Uniprot accession number P15907-1. In some embodiments the sialyltransferase has the amino acid set out in SEQ ID NO. 1. In some embodiments the sialyltransferase has the amino acid set out in SEQ ID NO. 4. In some embodiments the sialyltransferase has the amino acid set out in SEQ ID NO. 7. In some embodiments the sialyltransferase is a polypeptide having sialyltransferase activity and comprising a sequence having at least 70% sequence identity to SEQ ID NO. 1, 4, or 7, such as at least 80%, at least 90%, at least 95%, at least 98%, or 100% identity to SEQ ID NO. 1, 4, or 7.

The methods of preparing a glycoconjugate described herein also recite the use in step (b) above of a galactosyltransferase. In some embodiments the galactosyltransferase is human beta-1,4-galactosyltransferase 1 (B4GalT1). In some embodiments the galactosyltransferase has the amino acid disclosed in Uniprot accession number P15291-1. In some embodiments the galactosyltransferase has the amino acid set out in SEQ ID NO. 2. In some embodiments the sialyltransferase has the amino acid set out in SEQ ID NO. 5. In some embodiments the sialyltransferase has the amino acid set out in SEQ ID NO. 8. In some embodiments the sialyltransferase is a polypeptide having sialyltransferase activity and comprising a sequence having at least 70% sequence identity to SEQ ID NO. 2, 5, or 8, such as at least 80%, at least 90%, at least 95%, at least 98%, or 100% identity to SEQ ID NO. 2, 5, or 8.

Glycosidases

The methods of preparing a glycosylated cell-binding agent described herein also recite the use in step (c) above of a glycosidase. In some embodiments the glycosidase is an endoglycosidase.

In some embodiments the endoglycosidase is Endo S as disclosed in Collin, M. and Olsén, A. (2001). The EMBO Journal. 20, 3046-3055 [DOI: 10.1093/emboj/20.12.3046]). In some embodiments the endoglycosidase has the amino acid disclosed in Uniprot accession number Q9APG4-1. In some embodiments the endoglycosidase has the amino acid set out in SEQ ID NO. 3. In some embodiments the sialyltransferase has the amino acid set out in SEQ ID NO. 6. In some embodiments the sialyltransferase has the amino acid set out in SEQ ID NO. 9. In some embodiments the sialyltransferase is a polypeptide having sialyltransferase activity and comprising a sequence having at least 70% sequence identity to SEQ ID NO. 3, 6, or 9, such as at least 80%, at least 90%, at least 95%, at least 98%, or 100% identity to SEQ ID NO. 3, 6, or 9.

In some embodiments, the remodeling is performed using an endoglycosidase, for instance endoglycosidases classified into EC3.2.1.96. In some embodiments, the endoglycosidase includes endo-β-N-acetylglucosaminidase D (endoglycosidase D, Endo-D, or endo-D), endo-β-N-acetylglucosaminidase H (endoglycosidase H, Endo-H, or endo-H), endoglycosidase S (EndoS, Endo-S, or endo-S), endo-β-N-acetylglucosaminidase M (endoglycosidase M, Endo-M, or endo-M), endo-β-N-acetylglucosaminidase LL (endoglycosidase LL, EndoLL, Endo-LL, or endo-LL), endo-β-N-acetylglucosaminidase F1 (endoglycosidase F1, Endo-F1, or endo-F1), endo-β-N-acetylglucosaminidase F2 (endoglycosidase F2, Endo-F2, or endo-F2), and endo-β-N-acetylglucosaminidase F3 (endoglycosidase F3, Endo-F3, or endo-F3).

In some embodiments a combination of two or more types of endoglycosidases can be used in the remodeling step. For example, several endoglycosidases can be a combination of endoglycosidases having different substrate specificity that are classified into EC3.2.1.96. Exemplary combinations include endoglycosidase D and endoglycosidase S; endoglycosidase S and endoglycosidase LL; endoglycosidase D and endoglycosidase LL; endoglycosidase D and endoglycosidase H; endoglycosidase S and endoglycosidase H; endoglycosidase F1 and endoglycosidase F2; endoglycosidase F1 and endoglycosidase F3; endoglycosidase F2 and endoglycosidase F3; endoglycosidase D, endoglycosidase S and endoglycosidase LL; endoglycosidase D, endoglycosidase S and endoglycosidase H, and endoglycosidase D, endoglycosidase S and endoglycosidase F1.

The remodeling may be carried out in a suitable buffer solution, such as for example phosphate, buffered saline (e.g. phosphate-buffered saline, tris-buffered saline), citrate, HEPES, tris and glycine. Suitable buffers are known in the art. Preferably, the buffer solution is phosphate-buffered saline (PBS) or tris buffer. The remodeling is preferably performed at a temperature in the range of about 4 to about 50° C., more preferably in the range of about 10 to about 45° C., even more preferably in the range of about 20 to about 40° C., and most preferably in the range of about 30 to about 37° C. The remodeling can be carried out at a pH in the range of about 5 to about 9, preferably in the range of about 5.5 to about 8.5, more preferably in the range of about 6 to about 8. Most preferably, the process is performed at a pH in the range of about 7 to about 8.

Carbohydrate Moiety

In an oligoglycosylated cell-binding agent of the formula cell-binding agent having the formula:

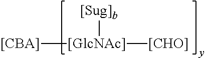

The element [CHO] represents a carbohydrate moiety. The carbohydrate moiety may be a monosaccharide, disaccharide, trisaccharide, or longer oligomer of polymer of sugars or sugar derivatives. The constituent sugar or sugar derivative units may be linked to each other in any orientation of linkage, and may form structures with two, three, or more branches.

In a fourth aspect the present disclosure provides for the use of the glycosylated cell-binding agent as defined herein in the production of a glycoconjugate as defined herein.

Typically, in embodiments where the CBA is an antibody, the carbohydrate moiety represents the remainder of the native N-linked glycan present on the antibody prior to the remodelling methods described herein.

The reaction between the glycosylated cell-binding agent and compound of the formula payload-G^{L} may be performed in an aqueous buffer solution, such as for example phosphate, buffered saline (e.g. phosphate-buffered saline, tris-buffered saline), citrate, HEPES, tris and glycine. Preferably, the buffer solution is phosphate-buffered saline (PBS) or tris buffer.

The reaction may be carried out at a temperature between about 4 to about 50° C., more preferably between about 10 to about 45° C., even more preferably between about 20 to about 40° C., and most preferably in the range of about 30 to about 37° C. The reaction may be carried out at a pH in the range of about 5 to about 9, preferably in the range of about 5.5 to about 8.5, more preferably in the range of about 6 to about 8. Most preferably, the reaction is carried out at a pH in the range of about 7 to about 8.

In certain cases, the first compound can include mixtures of the 2.6 and 2.3 linked glycosylated cell-binding agent described herein. In other embodiments, the glycosylated cell-binding agent can be substantially only the 2.6 linked oligosaccharide, or substantially on the 2.3 linked oligosaccharide. In some embodiments, the glycosylated cell-binding agent can be at least 90%, at least 95%, at least 98%, or at least 99% of the 2.6 linked oligosaccharide, while in other embodiments, the glycosylated cell-binding agent can be at least 90%, at least 95%, at least 98%, or at least 99% of the 2.3 linked oligosaccharide.

Methods of Treatment

The glycoconjugates described herein may be used in a method of therapy. Also provided is a method of treatment, comprising administering to a subject in need of treatment a therapeutically-effective amount of a glycoconjugate described herein. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A glycoconjugate described herein may be administered alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the glycoconjugates described herein include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Medical Uses

The glycoconjugates of the disclosure may be used to provide a payload at a target location.

In some preferred embodiments the target location is a proliferative cell population. In some preferred embodiments the CBA specifically binds a target antigen present on a proliferative cell population.

In one embodiment the target antigen is absent or present at a reduced level in a non-proliferative cell population compared to the amount of antigen present in the proliferative cell population, for example a tumour cell population.

At the target location the linker portion of the payload connecting the payload to the CBA may be cleaved so as to release the payload compound. Thus, the glycoconjugate may be used to selectively provide part or all of a payload compound to the target location.

The linker portion may be cleaved by an enzyme present at the target location.

The target location may be in vitro, in vivo or ex vivo.

The glycoconjugates of the present disclosure include those with utility for anticancer activity. In particular, glycoconjugates where the CBA (such as an antibody) is conjugated i.e. covalently attached by a linker, to a cytotoxic drug moiety, such as a PBD drug. Typically in these embodiments, the drug has a cytotoxic effect when it is released form the CBA. The biological activity of the drug moiety is thus modulated by conjugation to a CBA. The glycoconjugates of the disclosure can selectively deliver an effective dose of a cytotoxic agent to tumor tissue whereby greater selectivity, i.e. a lower efficacious dose, may be achieved.

Thus, in one aspect, the present invention provides a glycoconjugate as described herein for use in therapy.

In a further aspect there is also provides a glycoconjugate compound as described herein for use in the treatment of a proliferative disease. A second aspect of the present disclosure provides the use of a conjugate compound in the manufacture of a medicament for treating a proliferative disease.

One of ordinary skill in the art is readily able to determine whether or not a candidate glycoconjugate treats a proliferative condition for any particular cell type. For example, assays which may conveniently be used to assess the activity offered by a particular compound are described in the examples below.

The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, neuroblastoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, renal cancer, brain cancer, sarcoma, liposarcoma, osteosarcoma, Kaposi's sarcoma, melanoma), lymphomas, leukemias, myeloma, psoriasis, bone diseases, fibroproliferative disorders (e.g. of connective tissues), infectious disease, and atherosclerosis.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Disorders of particular interest include, but are not limited to cancers, including metastatic cancers and metastatic cancer cells, such as circulating tumour cells, which may be found circulating in body fluids such as blood or lymph. Cancers of particular interest include: Hepatocellular carcinoma, hepatoblastoma, non small cell lung cancer, small cell lung cancer, colon cancer, breast cancer, gastric cancer, pancreatic cancer, neuroblastoma, adrenal gland cancer, pheochromocytoma, paraganglioma, thyroid medullary carcinoma, skeletal muscle cancer, liposarcoma, glioma, Wilms tumor, neuroendocrine tumors, Acute Myeloid Leukemia and Myelodysplastic syndrome.

Other disorders of interest include any condition in which a target antigen is overexpressed, or wherein antagonism of a target antigen will provide a clinical benefit. These may include immune disorders, infectious disease, cardiovascular disorders, thrombosis, diabetes, immune checkpoint disorders, fibrotic disorders (fibrosis), or proliferative diseases such as cancer, particularly metastatic cancer.

Formulations

While it is possible for the glycoconjugate compound to be used (e.g., administered) alone, it is often preferable to present it as a composition or formulation.

In one embodiment, the composition is a pharmaceutical composition (e.g., formulation, preparation, medicament) comprising a glycoconjugate compound, as described herein, and a pharmaceutically acceptable carrier, diluent, or excipient.

In one embodiment, the composition is a pharmaceutical composition comprising at least one glycoconjugate compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

Another aspect of the present disclosure pertains to methods of making a pharmaceutical composition comprising admixing at least one [¹¹C]-radiolabelled conjugate or conjugate-like compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Dosage

It will be appreciated by one of skill in the art that appropriate dosages of the conjugate compound, and compositions comprising the conjugate compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, the active compound is administered to a human patient according to the following dosage regime: about 100 mg, 3 times daily.

In one embodiment, the active compound is administered to a human patient according to the following dosage regime: about 150 mg, 2 times daily.

In one embodiment, the active compound is administered to a human patient according to the following dosage regime: about 200 mg, 2 times daily.

However in one embodiment, the conjugate compound is administered to a human patient according to the following dosage regime: about 50 or about 75 mg, 3 or 4 times daily.

In one embodiment, the conjugate compound is administered to a human patient according to the following dosage regime: about 100 or about 125 mg, 2 times daily.

The dosage amounts described above may apply to the glycoconjugate (including the payload and/or the linker to the antibody) or to the effective amount of payload provided, for example the amount of payload that is releasable from the CBA (in cases where release of part or all of the payload is required for efficacy).

For the prevention or treatment of disease, the appropriate dosage of the glycoconjugates described herein will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 15 mg/kg (e.g. 0.1-20 mg/kg) of molecule is an initial candidate dosage for administration to the patient, whether, for example, by one or more separate administrations, or by continuous infusion. A typical daily dosage might range from about 1 μg/kg to 100 mg/kg or more, depending on the factors mentioned above. An exemplary dosage of glycoconjugate to be administered to a patient is in the range of about 0.1 to about 10 mg/kg of patient weight. For repeated administrations over several days or longer, depending on the condition, the treatment is sustained until a desired suppression of disease symptoms occurs. An exemplary dosing regimen comprises a course of administering an initial loading dose of about 4 mg/kg, followed by additional doses every week, two weeks, or three weeks of a glycoconjugate. Other dosage regimens may be useful. The progress of this therapy is easily monitored by conventional techniques and assays.

Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human.

Furthermore, the subject/patient may be any of its forms of development, for example, a foetus. In one preferred embodiment, the subject/patient is a human.

### Definitions

Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₁₂ Alkenyl: The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₁₂ alkynyl: The term “C₂₋₁₂ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);
unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and
saturated polycyclic hydrocarbon compounds:

norcarane (C₇), norpinane (C₇), norbornane (C₇).

C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms. In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:
N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. The term “C₅₋₇ aryl”, as used herein, pertains to a monovalent moiety obtained by removing a hydrogen atom from an aromatic ring atom of an aromatic compound, which moiety has from 5 to 7 ring atoms and the term “C₅₋₁₀ aryl”, as used herein, pertains to a monovalent moiety obtained by removing a hydrogen atom from an aromatic ring atom of an aromatic compound, which moiety has from 5 to 10 ring atoms. Preferably, each ring has from 5 to 7 ring atoms.

In this context, the prefixes (e.g. C₃₋₂₀, C₅₋₇, C₅₋₆, C₅₋₁₀, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”.

Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to:
C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ (with 2 fused rings) derived from benzodiazepine (N₂);
C₁₃ (with 3 fused rings) derived from carbazole (N₁), dibenzofuran (O₁), dibenzothiophene (S₁), carboline (N₂), perimidine (N₂), pyridoindole (N₂); and,
C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇alkyl group.

Alkoxy: —OR, wherein R is an alkyl group, for example, a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec—butoxy), —O(iBu) (isobutoxy), and —O(tBu) (tert-butoxy).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇ alkyl group, a C₃₋₂₀ heterocyclyl group, or a C₃₋₂₀ aryl group, preferably a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of imino groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃(t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

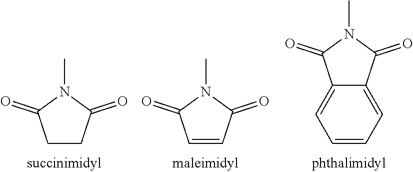

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

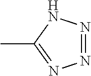

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇alkyl group. Examples of imino groups include, but are not limited to, ═NH, ═NMe, and ═NEt.

Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, where R is a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR)₂, where R is a phosphate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR)₂, where R is a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

Alkylene

C₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH—₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

Includes Other Forms

Unless otherwise specified, included in the definitions herein are the well-known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Salts

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic acid and valeric. Examples of suitable polymeric organic anions include, but are not limited to, those derived from the following polymeric acids: tannic acid, carboxymethyl cellulose.

Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

The disclosure describes PBD compounds where a solvent adds across the imine bond of the PBD moiety, which is illustrated below where the solvent is water or an alcohol (R^{A}OH, where R^{A} is C₁₋₄ alkyl):

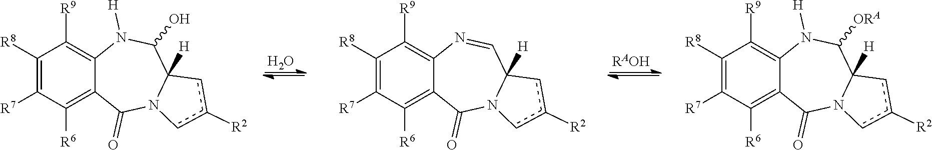

These forms can be called the carbinolamine and carbinolamine ether forms of the PBD. The balance of these equilibria depend on the conditions in which the compounds are found, as well as the nature of the moiety itself.

These particular compounds may be isolated in solid form, for example, by lyophilisation.

Isomers

Certain compounds described herein may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds described herein may contain asymmetric or chiral centers, and therefore exist in different stereoisomeric forms. It is intended that all stereoisomeric forms of the compounds described herein, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

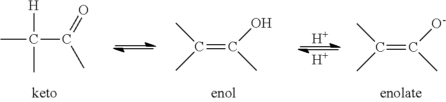

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into disclosed compounds include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled disclosed compounds, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic disclosed compounds may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled disclosed compounds and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the disclosed compounds any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Glycoforms

As used herein, the term ‘glycoform’ is used to refer to a glycosylated molecule (typically a glycoprotein) having a particular and specific glycosylation pattern. Accordingly, if two molecules are described as being the same glycoform both molecules have identical patterns of glycosylation, including location of glycan attachment as well as structure, composition, and linkage of the glycan structures themselves.

### BRIEF DESCRIPTION OF THE FIGURES

Embodiments and experiments illustrating the principles of the disclosure will now be discussed with reference to the accompanying figures in which:

FIG. 1.

Synthesis of PL1603

FIG. 2.

Glycosylation remodelling and conjugation according to Approach 1. GlcNAc=A-acetyl-glucosamine, Man=mannose, Gal=galactose, Fuc=fucose, Sia=sialic acid, PBD/DBP=PL1603. Reaction conditions:
(i) UDP-Galactose, galactosyltransferase, MOPS buffer (50 mM, 20 mM MnCl2, pH 7.2), 24 h;
(ii) CMP-Neu5N₃, ST6Gal1 sialyltransferase, 1% BSA, alkaline phosphatase, cacodylate buffer (50 mM, pH 7.6), 24 h;
(iii) PL1603.

FIG. 3.

Glycosylation remodelling and conjugation according to Approach 2. GlcNAc=A-acetyl-glucosamine, Man=mannose, Gal=galactose, Fuc=fucose, Sia=sialic acid, PBD/DBP=PL1603. Reaction conditions:
(i-1) EndoS;
(i-2) EndoS/BtFucH;
(ii) UDP-Galactose, β4GalT1, MOPS buffer (50 mM, 20 mM MnCl2, pH 7.2), 1% BSA, 1.3% alkaline phosphatase;
(iii) CMP-Neu5N3, cacodylate buffer (50 mM, pH 7.6), 1% BSA, 1.3% alkaline phosphatase;
(iv) PL1603.

FIG. 4.

HIC profile of Her-PL1603-App1 and Her-PL1603-App2

FIG. 5.

In vivo efficacy of Her-PL1603-App1 and Her-PL1603-App2 versus the benchmark Her2×ADC

FIG. 6.

Pharmacokinetics (PK) of Her-PL1603-App1 and Her-PL1603-App2 in rats

### STATEMENTS OF INVENTION

1. A glycoconjugate having the formula:

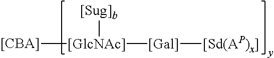

wherein:
CBA is a Cell Binding Agent;
GlcNAc is a N-acetyl-glucosamine moiety;
Sug is a sugar moiety, wherein b=1 or 0;
Gal is a galactose moiety;
Sd(A^{P})ₓ is a sugar derivative comprising x conjugated payloads A, wherein x=1, 2, 3, or 4;
wherein y=1 to 20; and
wherein the payload A^{P} is, comprises, or releases upon metabolism, a PBD compound, such as a PBD dimer;
optionally wherein GlcNAc, Gal, Sug, and/or Sd(A^{P})ₓ are D enantiomers.

2. The glycoconjugate of statement 1, wherein the PBD compound is a compound of formula I:

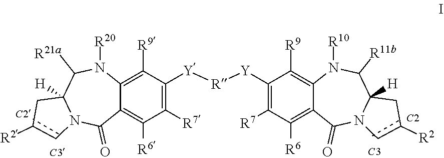

wherein:
[C6]
R⁶ is selected from H, R, OH, OR, SH, SR, NH₂, NHR, NRR′, nitro, Me₃Sn and halo;
R^{6′} is selected from H, R, OH, OR, SH, SR, NH₂, NHR, NRR′, nitro, Me₃Sn and halo;
[C9]
R⁹ is selected from H, R, OH, OR, SH, SR, NH₂, NHR, NRR′, nitro, Me₃Sn and halo;
R^{9′} is selected from H, R, OH, OR, SH, SR, NH₂, NHR, NRR′, nitro, Me₃Sn and halo;
[Y]
Y is selected from O, S, or NH;
Y′ is selected from O, S, or NH;
[C2]
when there is a double bond present between C2 and C3, R² is selected from the group consisting of:
(ia) C₅₋₁₀ aryl group, optionally substituted by one or more substituents selected from the group comprising: halo, nitro, cyano, hydroxy, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

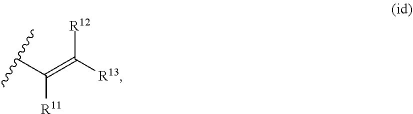

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

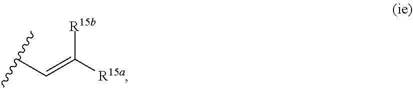

wherein one of R^{15a} and R^{15b} is H and the other is selected from: phenyl, which phenyl is optionally substituted by a group selected from halo, methyl, methoxy; pyridyl; and thiophenyl; and

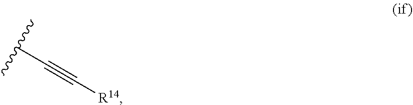

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is selected from H, OH, F, diF and

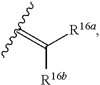

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
[C2′]
when there is a double bond present between C2′ and C3′, R^{2′} is selected from the group consisting of:
(iia) C₅₋₁₀ aryl group, optionally substituted by one or more substituents selected from the group comprising: halo, nitro, cyano, hydroxy, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl;

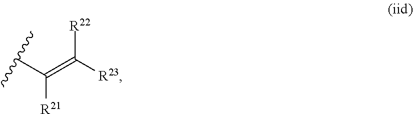

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

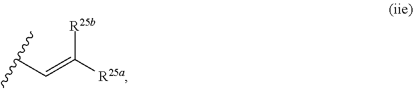

wherein one of R^{25a} and R^{25b} is H and the other is selected from: phenyl, which phenyl is optionally substituted by a group selected from halo, methyl, methoxy; pyridyl; and thiophenyl; and

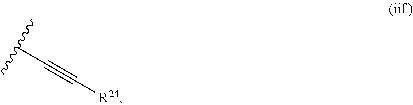

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R^{2′} is selected from H, OH, F, diF and

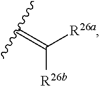

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
[C7]
R⁷ is selected from H, R, OH, OR, SH, SR, NH₂, NHR, NHRR′, nitro, Me₃Sn and halo;
R^{7′} is selected from H, R, OH, OR, SH, SR, NH₂, NHR, NHRR′, nitro, Me₃Sn and halo;
[R″]
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
[B10-C11]
(a) R¹⁰ is H, and R^{11a} is OH or OR^{A}, where R^{A} is C₁₋₄ alkyl; or
(b) R¹⁰ and R^{11a} form a nitrogen-carbon double bond between the nitrogen and carbon atoms to which they are bound; or
(c) R¹⁰ is H and R^{11a} is SO_{z}M, where z is 2 or 3 and M is a monovalent pharmaceutically acceptable cation; or
(d) R¹⁰ is H and R^{11a} is H; or
(e) R^{11a} is OH or OR^{A}, where R^{A} is C₁₋₄ alkyl and R¹⁰ is selected from:

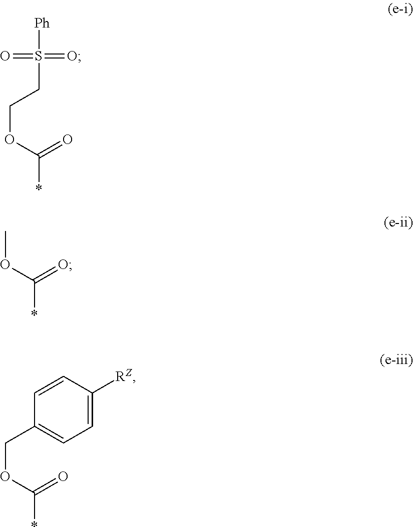

where R^{Z} is selected from:

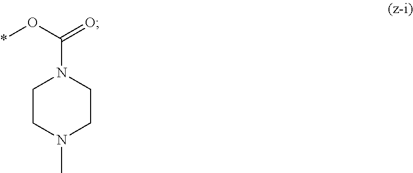 [SMILES: *OC(=O)N1CCN(C)CC1]

(z-ii) OC(═O)CH₃;
(z-iii) NO₂;
(z-iv) OMe;
(z-v) glucuronide;
(z-vi) —C(═O)—X₁—NHC(═O)X₂—NH—R^{ZC}, where —C(═O)—X₁—NH— and —C(═O)—X₂—NH— represent natural amino acid residues and R^{ZC} is selected from Me, OMe, OCH₂CH₂OMe;
(a) R²⁰ is H, and R^{21a} is OH or OR^{A}, where R^{A} is C₁₋₄ alkyl; or
(b) R²⁰ and R^{21a} form a nitrogen-carbon double bond between the nitrogen and carbon atoms to which they are bound; or
(c) R²⁰ is H and R^{21a} is SO_{z}M, where z is 2 or 3 and M is a monovalent pharmaceutically acceptable cation; or
(d) R²⁰ is H and R^{21a} is H; or
(e) R^{21a} is OH or OR^{A}, where R^{A} is C₁₋₄ alkyl and R²⁰ is selected from:

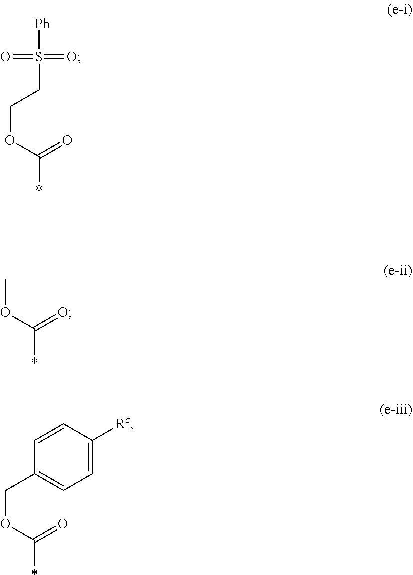

where R^{Z} is selected from:

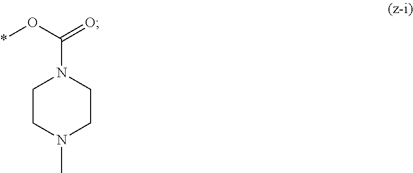 [SMILES: *OC(=O)N1CCN(C)CC1]

(z-ii) OC(═O)CH₃;
(z-iii) NO₂;
(z-iv) OMe;
(z-v) glucuronide;
(z-vi) —C(═O)—X₁—NHC(═O)X₂—NH—R^{ZC}, where —C(═O)—X₁—NH— and —C(═O)—X₂—NH— represent natural amino acid residues and R^{ZC} is selected from Me, OMe, and OCH₂CH₂OMe.

3. The glycoconjugate of either one of statements 1 or 2, wherein b=0.

4. The glycoconjugate of either one of statements 1 or 2, wherein b=1.

5. The glycoconjugate of any preceding statement, wherein Sd(A^{P})x is a sialic acid derivative.

6. The glycoconjugate of statement 5, wherein the sialic acid derivative has the formula:

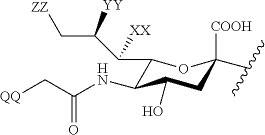

wherein:
QQ is hydrogen or a conjugated payload;
ZZ is hydroxyl or a conjugated payload;
YY is hydroxyl or a conjugated payload; and/or
XX is hydroxyl or a conjugated payload;

and wherein at least one of QQ, ZZ, YY, and XX is a conjugated payload.

7. The glycoconjugate of any preceding statement having the the formula:

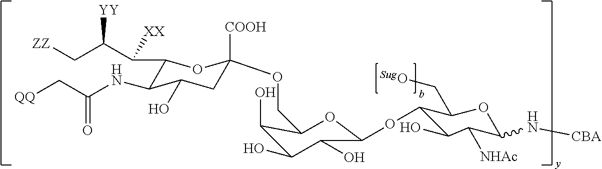

wherein:
QQ is hydrogen or a conjugated payload;
ZZ is hydroxyl or a conjugated payload;
YY is hydroxyl or a conjugated payload; and/or
XX is hydroxyl or a conjugated payload;

and wherein at least one of QQ, ZZ, YY, and XX is a conjugated payload

8. The glycoconjugate of statement 7, wherein the GlcNAc moiety is bonded to the CBA with an α-N-glycosidic linkage and has the formula:

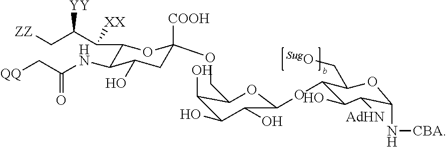

9. The glycoconjugate of statement 7, wherein the cell-binding agent is a protein, or comprises a protein portion, and wherein the GlcNAc moiety is conjugated to the cell-binding agent through an asparagine side chain via an α-N-glycosidic bond with the formula:

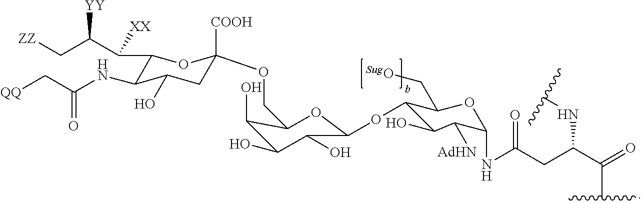

10. The glycoconjugate of any one of statements 1 to 6 having the the formula:

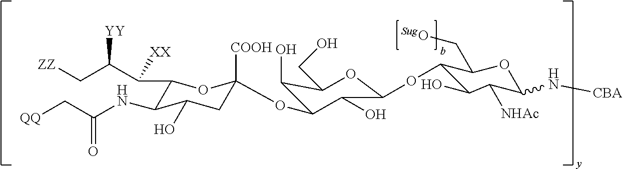

wherein:
QQ is hydrogen or a conjugated payload;
ZZ is hydroxyl or a conjugated payload;
YY is hydroxyl or a conjugated payload; and/or
XX is hydroxyl or a conjugated payload;

and wherein at least one of QQ, ZZ, YY, and XX is a conjugated payload.

11. The glycoconjugate of statement 10, wherein the GlcNAc moiety is bonded to the CBA with an α-N-glycosidic linkage and has the formula:

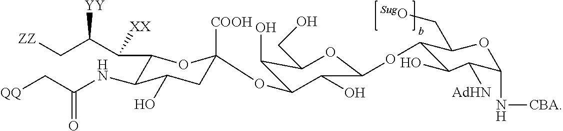

12. The glycoconjugate of statement 10, wherein the cell-binding agent is a protein, or comprises a protein portion, and wherein the GlcNAc moiety is conjugated to the cell-binding agent through an asparagine side chain via an α-N-glycosidic bond with the formula:

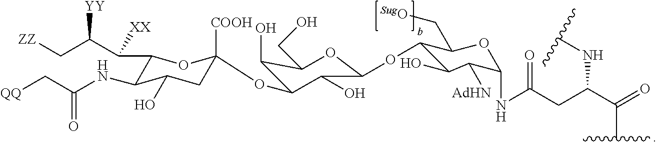

13. The glycoconjugate of any preceding statement, wherein Sug is a fucose moiety.

14. The glycoconjugate of any preceding statement, wherein Sug is a fucose moiety α1-6 linked to the GlcNAc moiety.

15. The glycoconjugate of either one of statements 13 or 14, wherein the fucose moiety has the structure:

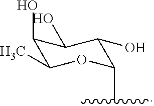 [SMILES: *C1OC(C)C(O)C(O)C1O]

16. The glycoconjugate of any one of statements 6 to 15 having a conjugated payload at position QQ.

17. The glycoconjugate of any one of statements 6 to 16 having a conjugated payload at position ZZ.

18. The glycoconjugate of any preceding statement, wherein x=1.

19. The glycoconjugate of any preceding statement having a conjugated payload at each of positions QQ and ZZ;
optionally wherein QQ and ZZ are the same.

20. The glycoconjugate of any one of statements 1 to 19, wherein x=2.

21. The glycoconjugate of any preceding statement, wherein y=1, 2, 3, or 4.

22. The glycoconjugate of any preceding statement, wherein y=2.

23. The glycoconjugate of any preceding statement, wherein y=1 to 2, 1 to 3, 2 to 4, 3-6 or 4-8.

24. The glycoconjugate of any preceding statement, wherein the CBA is a protein.

25. The glycoconjugate of statement 24, wherein the protein is a therapeutic protein.

26. The glycoconjugate of any preceding statement, wherein the CBA is a Fc fusion protein.

27. The glycoconjugate of statement 26, wherein the Fc domain is of the IgG isotype.

28. The glycoconjugate of either one of statements 26 or 27, wherein the Fc domain is of the IgG1 subclass.

29. The glycoconjugate of any preceding statement, wherein the CBA is an antibody.

30. The glycoconjugate of statement 29, wherein the antibody is monoclonal.

31. The glycoconjugate of either one of statements 29 or 30, wherein the antibody is of the IgG isotype.

32. The glycoconjugate of any one of statements 29 to 31, wherein the antibody is of the IgG1 subclass.

33. The glycoconjugate of any one of statements 29 to 32, wherein the GlcNAc moiety is conjugated to the antibody at the asparagine 297 (Asn297) residue according to the EU index as set forth in Kabat.

34. The glycoconjugate of any one of statements 29 to 33, wherein the antibody is an intact antibody.

35. The glycoconjugate of any preceding statement, wherein the GlcNAc moiety is conjugated to the CBA via the GlcNAc C1 carbon.

36. The glycoconjugate of any preceding statement, wherein the CBA-N-GlcNAc linkage is in the beta anomeric configuration.

37. The glycoconjugate of any one of statements 24 to 36, wherein the GlcNAc moiety is α-linked to an asparagine residue in the protein backbone.

38. The glycoconjugate of any preceding statement, wherein the CBA specifically binds a target antigen selected from the group comprising of: BMPR1B, E16, STEAP1, 0772P, MPF, Napi3b, Sema 5b, PSCA hIg, ETB, MSG783, STEAP2, TrpM4, CRIPTO, CD21, CD79b, FcRH2, HER2, NCA, MDP, IL20R-alpha, Brevican, EphB2R, ASLG659, PSCA, GEDA, BAFF-R, CD22, CD79a, CXCR5, HLA-DOB, P2X5, CD72, LY64, FcRH1, IRTA2, TENB2, PSMA, SST, ITGAV, ITGB6, CEACAM5, MET, MUC1, CA9, EGFRvIII, CD33, CD19, IL2RA, AXL, CD30, BCMA, CT Ags, CD174, CLEC14A, GRP78-HSPA5, CD70, Stem Cell specific antigens, ASG-5, ENPP3, PRR4, GCC-GUCY2C, Liv-1-SLC39A6, 5T4, CD56-NCMA1, CanAg, FOLR1, GPNMB, TIM-1-HAVCR1, RG-1, B7-H4-VTCN1, PTK7, CD37, CD138, CD74, Claudins, EGFR, Her3, RON-MST1R, EPHA2, CD20-MS4A1, Tenascin C-TNC, FAP, DKK-1, CD52, CS1-SLAMF7, Endoglin, Annexin A1, V-CAM (CD106), DLK-1, KAAG1, IL13RA2, Endosialin, CD48, LRRC15, SLAMF6, and PLAC1.

39. The glycoconjugate of any preceding statement, wherein the payload is, comprises, or releases upon metabolism a PBD compound selected from the group consisting of:

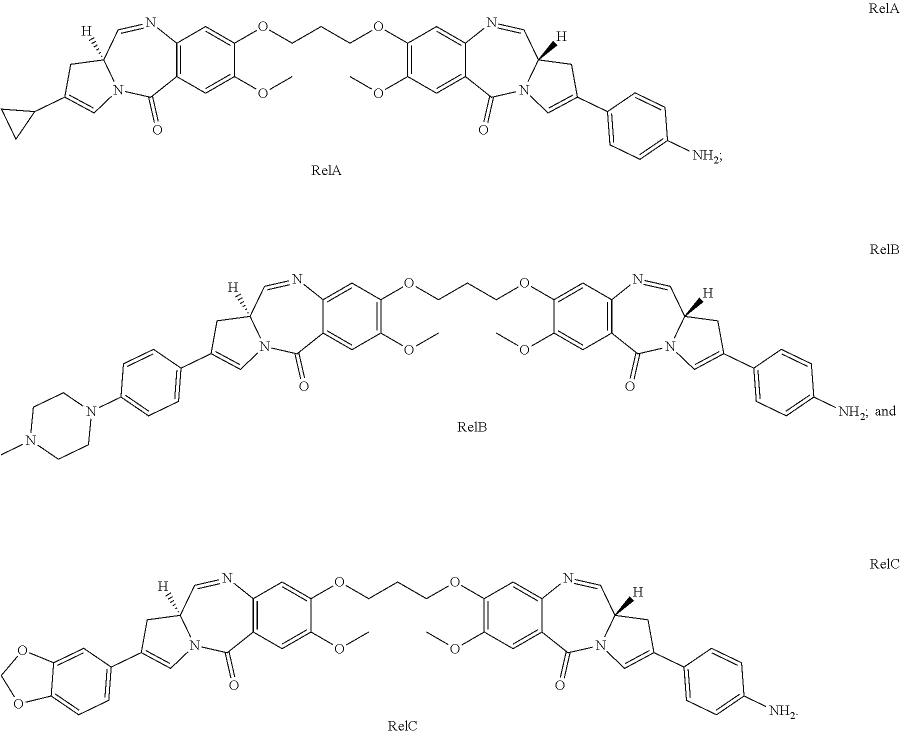 [SMILES: COc1cc2c(cc1OCCCOc1cc3c(cc1OC)C(=O)N1C=C(C4CC4)C[C@H]1C=N3)N=C[C@@H]1CC(c3ccc(N)cc3)=CN1C2=O ; COc1cc2c(cc1OCCCOc1cc3c(cc1OC)C(=O)N1C=C(c4ccc(N5CCN(C)CC5)cc4)C[C@H]1C=N3)N=C[C@@H]1CC(c3ccc(N)cc3)=CN1C2=O ; COc1cc2c(cc1OCCCOc1cc3c(cc1OC)C(=O)N1C=C(c4ccc5c(c4)OCO5)C[C@H]1C=N3)N=C[C@@H]1CC(c3ccc(N)cc3)=CN1C2=O]

40. The glycoconjugate of any preceding statement, wherein the payload is, comprises, or releases upon metabolism a PBD compound having the formula of RelD:

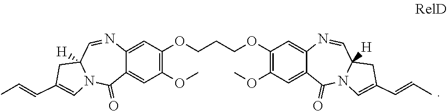 [SMILES: C/C=C/C1=CN2C(=O)c3cc(OC)c(OCCCOc4cc5c(cc4OC)C(=O)N4C=C(/C=C/C)C[C@H]4C=N5)cc3N=C[C@@H]2C1]

41. The glycoconjugate of any preceding statement, wherein the payload is, comprises, or releases upon metabolism a PBD compound having the formula of RelE:

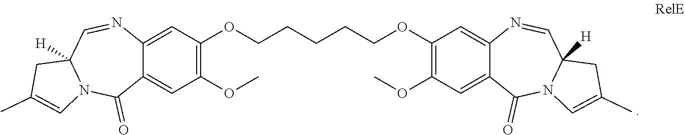

42. The glycoconjugate of any preceding statement, wherein the payload is, comprises, or releases upon metabolism a PBD compound having the formula of RelF:

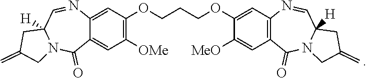

43. The glycoconjugate of any preceding statement, wherein the payload is, comprises, or releases upon metabolism a PBD compound having the formula of RelG:

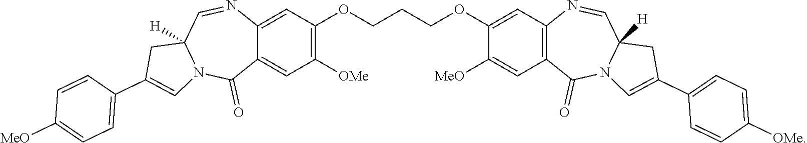 [SMILES: COc1ccc(C2=CN3C(=O)c4cc(OC)c(OCCCOc5cc6c(cc5OC)C(=O)N5C=C(c7ccc(OC)cc7)C[C@H]5C=N6)cc4N=C[C@@H]3C2)cc1]

44. The glycoconjugate of any preceding statement, wherein the payload is, comprises, or releases upon metabolism a PBD compound having the formula of RelH:

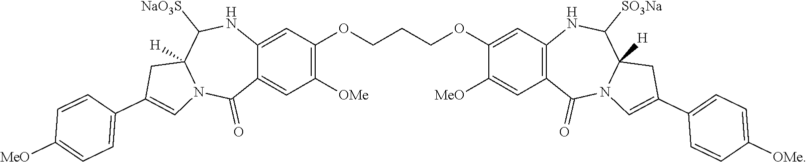

45. The glycoconjugate of any preceding statement, wherein the payload has a linker moiety linking the CBA and the remainder of the payload.

46. The glycoconjugate of any one of statements 1 to 45, wherein the conjugated payload, A^{P}, is a drug-linker comprising a drug moiety conjugated to the cell-binding agent via a linker moiety, the glycoconjugate having the formula:

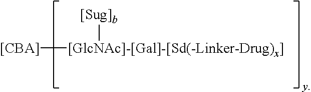

47. The glycoconjugate of statement 46, wherein -[Sd(-Linker-Drug)ₓ] has the formula:

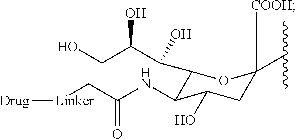

wherein the wavy line indicates where the Sd moiety is bound to the Gal moiety.

48. The glycoconjugate of statement 46, wherein -[Sd(-Linker-Drug)ₓ] has the formula:

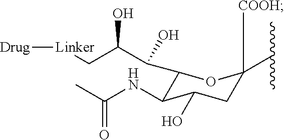

wherein the wavy line indicates where the Sd moiety is bound to the Gal moiety.

49. The glycoconjugate of any one of statements 46 to 48, wherein each drug-linker independently has a formula selected from the group consisting of:

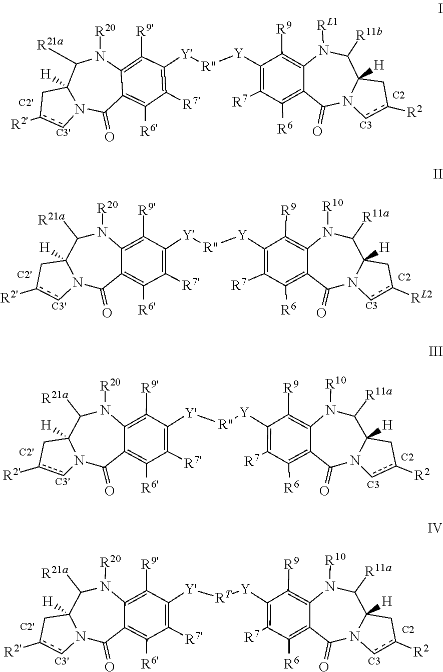

wherein:
R⁶ is selected from H, R, OH, OR, SH, SR, NH₂, NHR, NRR′, nitro, Me₃Sn and halo;
R^{6′} is selected from H, R, OH, OR, SH, SR, NH₂, NHR, NRR′, nitro, Me₃Sn and halo;
R⁹ is selected from H, R, OH, OR, SH, SR, NH₂, NHR, NRR′, nitro, Me₃Sn and halo;
R^{9′} is selected from H, R, OH, OR, SH, SR, NH₂, NHR, NRR′, nitro, Me₃Sn and halo;
Y is selected from O, S, or NH;
Y′ is selected from O, S, or NH;
when there is a double bond present between C2 and C3, R² is selected from the group consisting of:
(ia) C₅₋₁₀ aryl group, optionally substituted by one or more substituents selected from the group comprising: halo, nitro, cyano, hydroxy, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

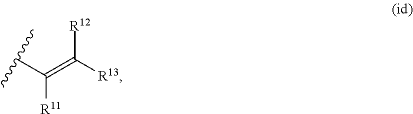

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

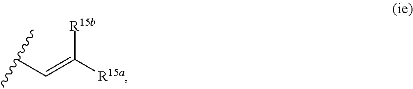

wherein one of R^{15a} and R^{15b} is H and the other is selected from: phenyl, which phenyl is optionally substituted by a group selected from halo, methyl, methoxy; pyridyl; and thiophenyl; and

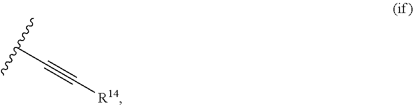

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
   R² is selected from H, OH, F, diF and

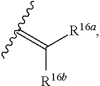

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

when there is a double bond present between C2′ and C3′, R^{2′} is selected from the group consisting of:
(iia) C₅₋₁₀ aryl group, optionally substituted by one or more substituents selected from the group comprising: halo, nitro, cyano, hydroxy, ether, carboxy, ester, C₁₋₇ alkyl, C₃₋₇ heterocyclyl and bis-oxy-C₁₋₃ alkylene;
(iib) C₁₋₅ saturated aliphatic alkyl;
(iic) C₃₋₆ saturated cycloalkyl;

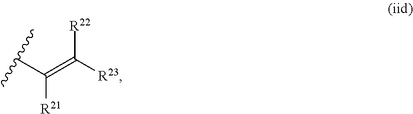

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

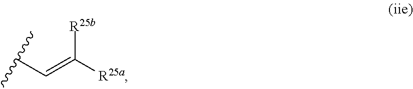

wherein one of R^{25a} and R^{25b} is H and the other is selected from: phenyl, which phenyl is optionally substituted by a group selected from halo, methyl, methoxy; pyridyl; and thiophenyl; and

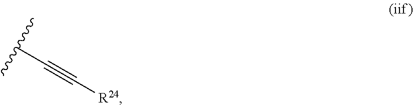

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R^{2′} is selected from H, OH, F, diF and

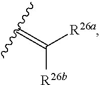

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁷ is selected from H, R, OH, OR, SH, SR, NH₂, NHR, NHRR′, nitro, Me₃Sn and halo;
R^{7′} is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
(a) R¹⁰ is H, and R^{11a} is OH or OR^{A}, where R^{A} is C₁₋₄ alkyl; or
(b) R¹⁰ and R^{11a} form a nitrogen-carbon double bond between the nitrogen and carbon atoms to which they are bound; or
(c) R¹⁰ is H and R^{11a} is SO_{z}M, where z is 2 or 3 and M is a monovalent pharmaceutically acceptable cation; or
(d) R¹⁰ is H and R^{11a} is H; or
(e) R^{11a} is OH or OR^{A}, where R^{A} is C₁₋₄ alkyl and R¹⁰ is selected from:

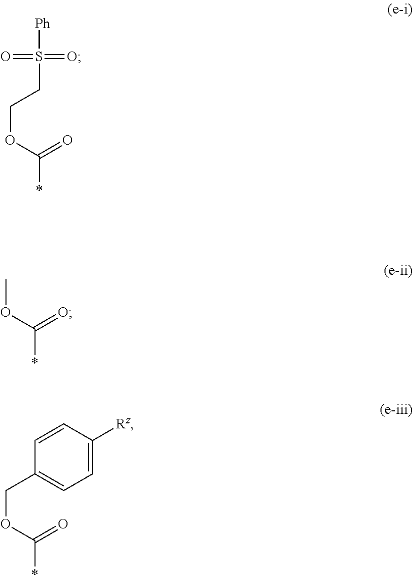

where R^{Z} is selected from:

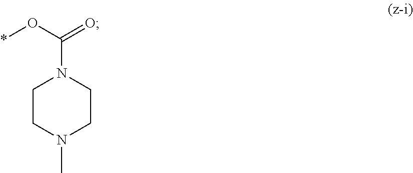 [SMILES: *OC(=O)N1CCN(C)CC1]

(z-ii) OC(═O)CH₃;
(z-iii) NO₂;
(z-iv) OMe;
(z-v) glucuronide;
   (z-vi) —C(═O)—X₁—NHC(═O)X₂—NH—R^{ZC}, where —C(═O)—X₁—NH— and —C(═O)—X₂—NH— represent natural amino acid residues and R^{ZC} is selected from Me, OMe, OCH₂CH₂OMe;
(a) R²⁰ is H, and R^{21a} is OH or OR^{A}, where R^{A} is C₁₋₄ alkyl; or
(b) R²⁰ and R^{21a} form a nitrogen-carbon double bond between the nitrogen and carbon atoms to which they are bound; or
(c) R²⁰ is H and R^{21a} is SO_{z}M, where z is 2 or 3 and M is a monovalent pharmaceutically acceptable cation; or
(d) R²⁰ is H and R^{21a} is H; or
(e) R^{21a} is OH or OR^{A} where R^{A} is C₁₋₄ alkyl and R²⁰ is selected from:

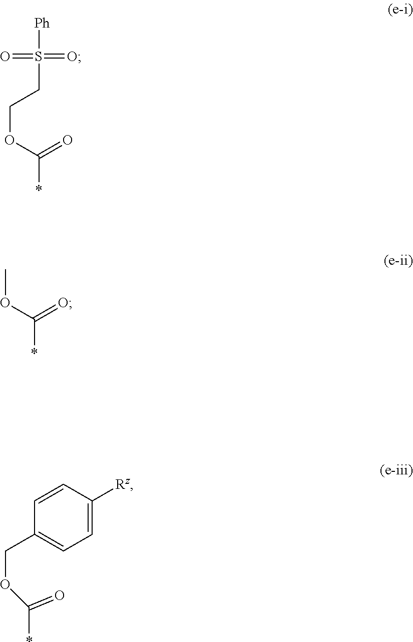

where R^{Z} is selected from:

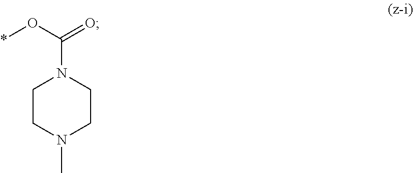 [SMILES: *OC(=O)N1CCN(C)CC1]

(z-ii) OC(═O)CH₃;
(z-iii) NO₂;
(z-iv) OMe;
(z-v) glucuronide;
(z-vi) —C(═O)—X₁—NHC(═O)X₂—NH—R^{ZC}, where —C(═O)—X₁—NH— and —C(═O)—X₂—NH— represent natural amino acid residues and R^{ZC} is selected from Me, OMe, OCH₂CH₂OMe;
R^{L1} and R^{11b}
R^{L1} is a linker for connection the Sd moiety;
R^{11b} is OH or OR^{A}, where R^{A} is C₁₋₄ alkyl;
R^{L2}
R^{L2} is of formula IIIa, formula IIIb or formula IIIc:

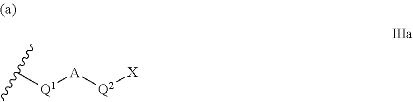

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

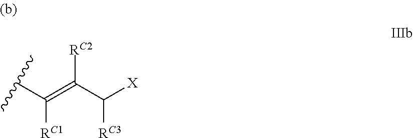

where;

R^{C1}, R^{C2} and R^{C3} are independently selected from H and unsubstituted C₁₋₂ alkyl;

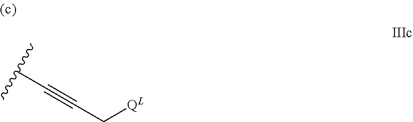

where Q^{L} is selected from O—R^{L2′}, S—R^{L2′} and NR^{N}—C(═O)—R^{L2′}, and R^{N} is selected from H, methyl and ethyl
X is selected from the group comprising: O—R^{L2′}, S—R^{L2′}, CO₂—R^{L2′}, CO—R^{L2′}, NR^{N}—C(═O)—R^{L2′}, NHNH—R^{L2′}, CONHNH—R^{L2′},

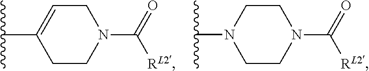

wherein R^{N} is selected from the group comprising H and C₁₋₄ alkyl;
R^{L2′} is a linker for connection to the Sd moiety;
R^{L3}
R^{L3} is selected from formulae A1, A2, A3, A4 and A5:

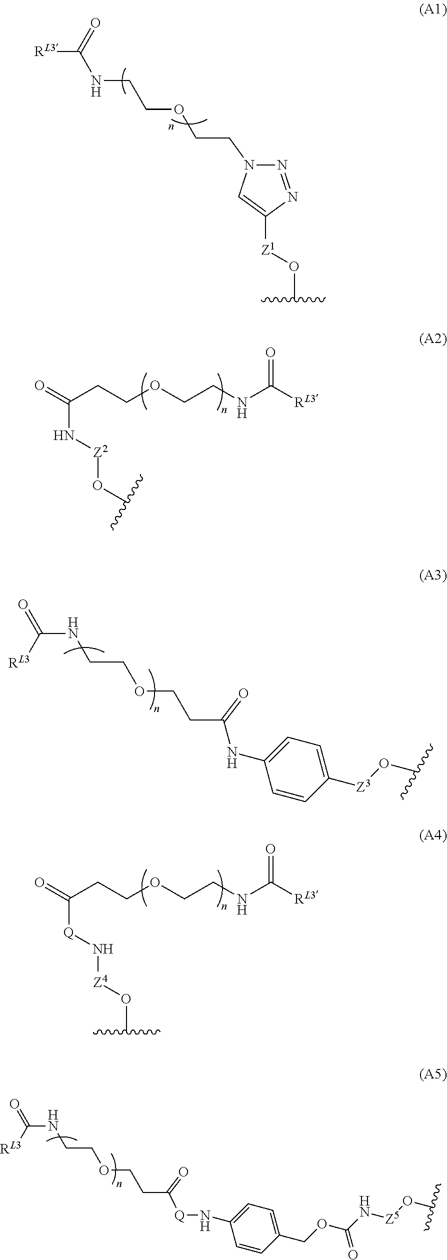

Z¹ is a C₁₋₃ alkylene group;
Z² is a C₁₋₃ alkylene group;
Z³ is a C₁₋₃ alkylene group;
Z⁴ is a C₁₋₃ alkylene group;
Z⁵ is a C₁₋₃ alkylene group;
n is an integer between 0 and 48;
Q is:

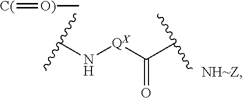

where Q^{X} is such that Q is an amino-acid residue, a dipeptide residue, a tripeptide residue, or a non-peptide moiety defined as PM in WO2015/095124;
R^{L3′} is a linker for connection to the Sd moiety;
R^{T}
R^{T} is:

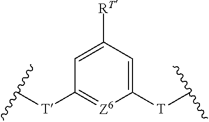

wherein
T and T″ are independently selected from a single bond or a C₁₋₉ alkylene, which chain may be interrupted by one or more heteroatoms e.g. O, S, N(H), NMe, provided that the number of atoms in the shortest chain of atoms between Y and Y′ is 3 to 12 atoms;
Z⁶ is CH or N;
R^{T′} is selected from formulae B1, B2, B3, B4, B5, B6, and B7:

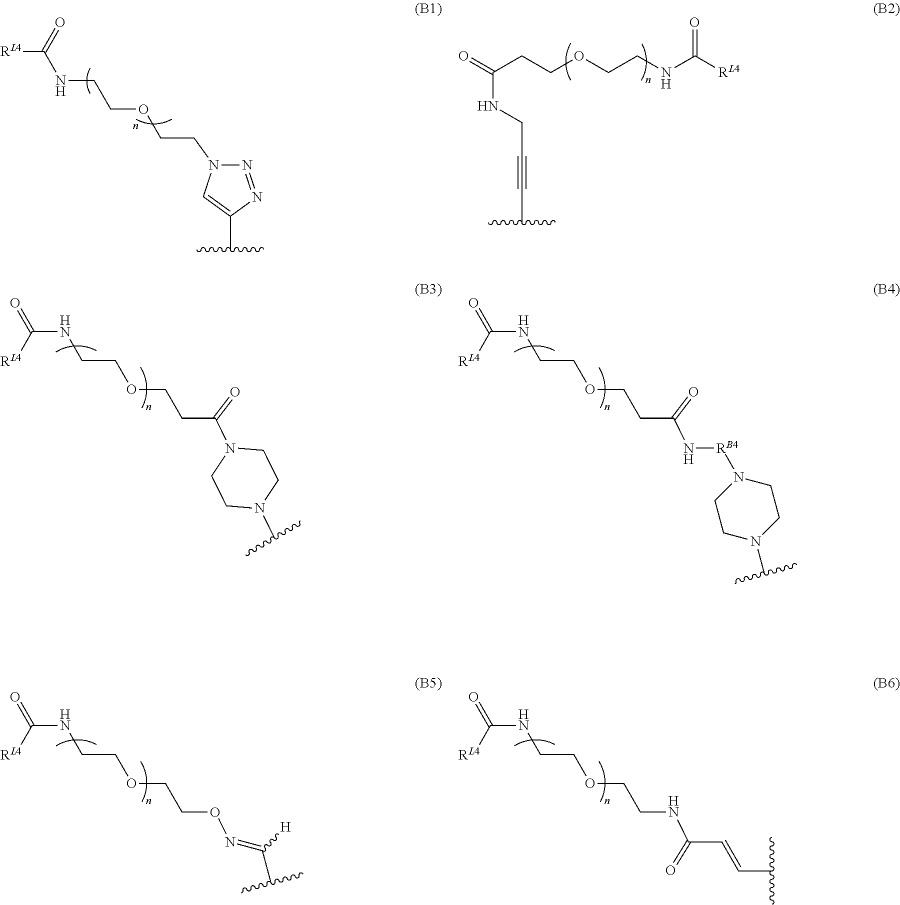

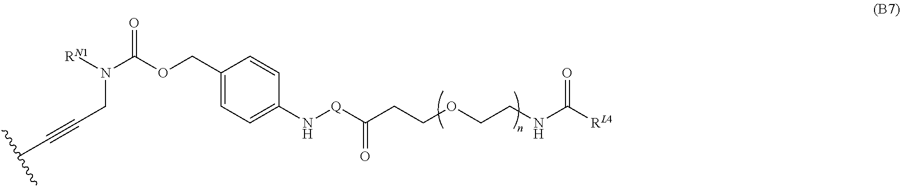

n is an integer selected in the range of 0 to 48;
R^{B4} is a C₁₋₆ alkylene group;
Q is:

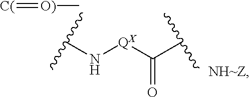

where Q^{X} is such that Q is an amino-acid residue, a dipeptide residue, a tripeptide residue, or a non-peptide moiety defined as PM in WO2015/095124;
R^{L4} is a linker for connection to the Sd moiety.

50. The glycoconjugate of any one of statements 46 to 48, wherein each linker independently is a linker of formula Z1 or Z2:

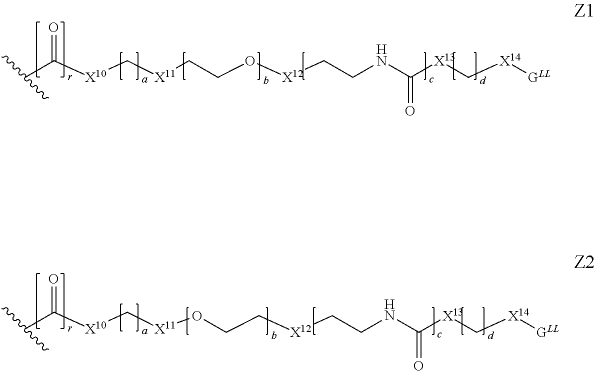

wherein r=0 or 1, a=0 to 5, b=0 to 16, c=0 or 1, d=0 to 5, G^{LL} is a linking moiety through which the linker is bound to the Sd moiety, the wavy line indicates where the linker is bound to the drug moiety, and one of X¹⁰, X¹¹, X¹², X¹³ and X¹⁴ may be selected from:

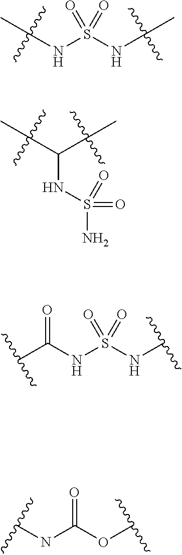 [SMILES: *NS(=O)(=O)N* ; *C(*)NS(N)(=O)=O ; *NS(=O)(=O)NC(*)=O ; *NC(=O)O*]

the remainder being a single bond.

51. The glycoconjugate of statements 50, wherein G^{LL} is selected from:

| | |
|---|---|
| (G^{LL8-1}) | 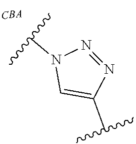 [SMILES: *c1cn(*)nn1] |
| G^{LL11} | 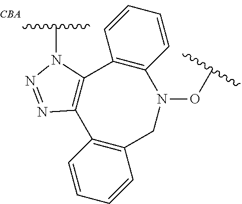 [SMILES: *ON1Cc2ccccc2-c2nnn(*)c2-c2ccccc21] |
| (G^{LL8-2}) | 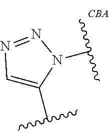 [SMILES: *c1cnnn1*] |
| G^{LL12} | 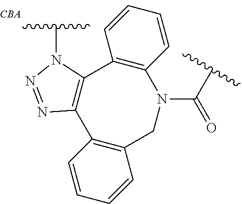 [SMILES: *C(=O)N1Cc2ccccc2-c2nnn(*)c2-c2ccccc21] |
| (G^{LL9-1}) | 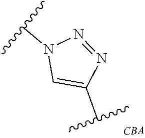 [SMILES: *c1cn(*)nn1] |
| G^{LL13} | 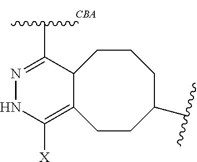 |
| (G^{LL9-2}) | 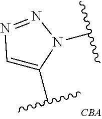 [SMILES: *c1cnnn1*] |
| G^{LL14} | 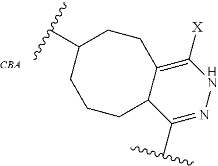 |
| G^{LL10} | 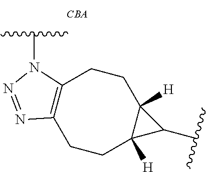 [SMILES: *C1[C@H]2CCc3c(nnn3*)CC[C@@H]12] |

where CBA indicates where the group is bound to Sd(A^{P})ₓ.

52. The glycoconjugate of any one of statements 46 to 51, wherein all the drug-linkers conjugated to the cell binding-agent are the same.

53. A method for the preparation of the glycoconjugate of any one of statements 1 to 52, the method comprising the steps of:
(i) providing a glycosylated cell-binding agent;
(ii) reacting the glycosylated cell-binding agent of (i) with a compound of the formula payload-G^{L}, wherein the payload is as defined in any one of statements 1 to 52 and G^{L} is linker group reactive with the functional group of the glycosylated cell-binding agent

54. The method of statement 53, wherein G^{L} is selected from the group consisting of:

| | |
|---|---|
| (G^{L8}) | 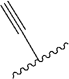 [SMILES: *C#C] |
| (G^{L9}) | 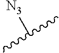 [SMILES: *N=[N+]=[N-]] |
| (G^{L10}) | 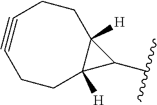 [SMILES: *C1[C@H]2CCC#CCC[C@@H]12] |
| (G^{L11}) | 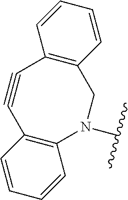 [SMILES: *N1Cc2ccccc2C#Cc2ccccc21] |
| (G^{L12}) | 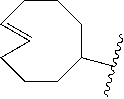 [SMILES: *C1CCC/C=C/CCC1] |
| (G^{L13}) | 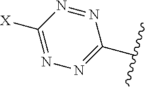 |

55. The method of either one of statements 53 or 54, wherein the provided glycosylated cell-binding agent is a glycosylated cell-binding agent having the formula:

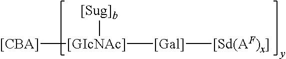

wherein:
CBA is a Cell Binding Agent;
GlcNAc is a N-acetyl-glucosamine moiety;
Sug is a sugar moiety, wherein b=1 or 0;
Gal is a galactose moiety;
Sd(A^{F})ₓ is a sugar derivative comprising x functional groups A^{F}, wherein A^{F} is independently selected from the group consisting of an azido group, an alkynyl group, and a keto group, and wherein x=1, 2, 3, or 4; and

wherein y=1 to 20.

56. The method of statement 55, wherein b=0.

57. The method of statement 55, wherein b=1.

58. The method any one of statements 55 to 57, wherein Sd(A^{F})ₓ is a sialic acid derivative.

59. The method of statement 58, wherein the sialic acid derivative has the formula:

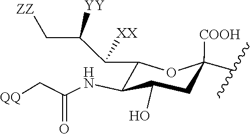

wherein:
QQ is hydrogen or a functional group A^{F};
ZZ is hydroxyl or a functional group A^{F};
YY is hydroxyl or a functional group A^{F}; and/or
XX is hydroxyl or a functional group A^{F};

and wherein at least one of QQ, ZZ, YY, and XX is a functional group A^{F}.

60. The method of any one of statements 55 to 59 wherein the glycosylated cell-binding agent of has the formula:

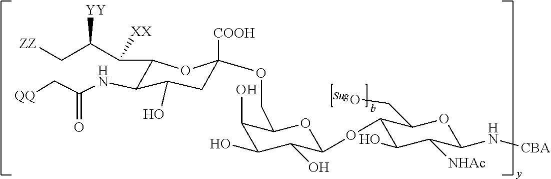

wherein:
QQ is hydrogen or a functional group A^{F};
ZZ is hydroxyl or a functional group A^{F};
YY is hydroxyl or a functional group A^{F}; and/or
XX is hydroxyl or a functional group A^{F};

and wherein at least one of QQ, ZZ, YY, and XX is a functional group A^{F}.

61. The method of any one of statements 53 to 60 wherein the glycosylated cell-binding agent of has the formula:

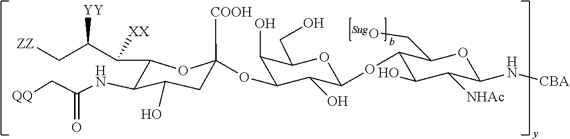

wherein:
QQ is hydrogen or a functional group A^{F};
ZZ is hydroxyl or a functional group A^{F};
YY is hydroxyl or a functional group A^{F}; and/or
XX is hydroxyl or a functional group A^{F};

and wherein at least one of QQ, ZZ, YY, and XX is a functional group A^{F}.

62. The method of any one of statements 55 to 61, wherein Sug is a fucose moiety.

63. The method of statement 62, wherein the fucose moiety has the structure:

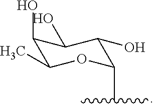 [SMILES: *C1OC(C)C(O)C(O)C1O]

64. The method of any one of statements 59 to 63, wherein the glycosylated cell-binding agent of has a functional group A^{F} at position QQ.

65. The method of any one of statements 59 to 64, wherein the glycosylated cell-binding agent of has a functional group A^{F} at position ZZ.

66. The method of any one of statements 55 to 65, wherein x=1.

67. The method of any one of statements 59 to 66, wherein the glycosylated cell-binding agent of has a functional group A^{F} at each of positions QQ and ZZ;
optionally wherein each of QQ and ZZ has the same functional group A^{F}.

68. The method of any one of statements 55 to 67, wherein x=2.

69. The method of any one of statements 55 to 68, wherein y=1, 2, 3, or 4.

70. The method of any one of statements 55 to 69, wherein y=2.

71. The method of any one of statements 55 to 70, wherein y=1 to 2, 1 to 3, 2 to 4, 3-6 or 4-8.

72. The method of any one of statements 55 to 71, wherein a functional group A^{F} is an azido group.

73. The method of any one of statements 55 to 72, wherein a functional group A^{F} is an alkynyl group.

74. The method of any one of statements 55 to 73, wherein the CBA is a protein.

75. The method of statement 74, wherein the protein is a therapeutic protein.

76. The method of any one of statements 55 to 75, wherein the CBA is an antibody.

77. The method of statement 76, wherein the antibody is monoclonal.

78. The method of either one of statements 76 or 77, wherein the antibody is of the IgG isotype.

79. The method of any one of statements 76 to 78, wherein the antibody is of the IgG1 subclass.

80. The method of any one of statements 76 to 79, wherein the GlcNAc moiety is conjugated to the antibody at the asparagine 297 (Asn297) residue according to the EU index as set forth in Kabat.

81. The method of any one of statements 76 to 80, wherein the antibody is an intact antibody.

82. The method of any one of statements 55 to 81, wherein the GlcNAc moiety is conjugated to the CBA via the GlcNAc C1 carbon.

83. The method of any one of statements 55 to 82, wherein the CBA-N-GlcNAc linkage is in the beta anomeric configuration.

84. The method of any one of statements 53 to 83, wherein the GlcNAc moiety is α-linked to an asparagine residue in the protein backbone.

85. The method of any one of statements 53 to 84, wherein the CBA specifically binds a target antigen selected from the group comprising of: BMPR1B, E16, STEAP1, 0772P, MPF, Napi3b, Sema 5b, PSCA hIg, ETB, MSG783, STEAP2, TrpM4, CRIPTO, CD21, CD79b, FcRH2, HER2, NCA, MDP, IL20R-alpha, Brevican, EphB2R, ASLG659, PSCA, GEDA, BAFF-R, CD22, CD79a, CXCR5, HLA-DOB, P2X5, CD72, LY64, FcRH1, IRTA2, TENB2, PSMA, SST, ITGAV, ITGB6, CEACAM5, MET, MUC1, CA9, EGFRvIII, CD33, CD19, IL2RA, AXL, CD30, BCMA, CT Ags, CD174, CLEC14A, GRP78-HSPA5, CD70, Stem Cell specific antigens, ASG-5, ENPP3, PRR4, GCC-GUCY2C, Liv-1-SLC39A6, 5T4, CD56-NCMA1, CanAg, FOLR1, GPNMB, TIM-1-HAVCR1, RG-1, B7-H4-VTCN1, PTK7, CD37, CD138, CD74, Claudins, EGFR, Her3, RON-MST1R, EPHA2, CD20-MS4A1, Tenascin C-TNC, FAP, DKK-1, CD52, CS1-SLAMF7, Endoglin, Annexin A1, V-CAM (CD106), DLK-1, KAAG1, IL13RA2, Endosialin, CD48, LRRC15, SLAMF6, and PLAC1.

86. A composition consisting of population of glycoconjugates according to any one of statements 1 to 52, wherein at least 75% of the molecules making up the population are the same glycoform.

87. A composition according to statement 86, wherein at least 80%, at least 85%, at least 90%, at least 95%, at least 97.5%, or at least 99% of the molecules making up the population are the same glycoform.

88. The method of any one of statements 55 to 85, wherein the glycosylated cell-binding agent is provided by a method comprising the steps of:
(i) providing a Sd(A^{F})ₓ acceptor having the formula:

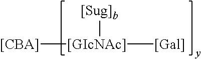

wherein CBA, GlcNAc, Sug, b, Gal, and y are defined as in of any one of statements 55 to 85; and
(ii) contacting the Sd(A^{F})ₓ acceptor with a compound of the formula Sd(A^{F})ₓ-P* in the presence of a glycosyltransferase, wherein:
Sd(A^{F})ₓ is as defined in any one of statements 55 to 85; and
P* is a nucleoside phosphate moiety.

89. The method of statement 88, wherein the Sd(A^{F})ₓ acceptor has the formula:

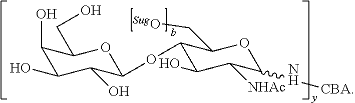

90. The method of statement 86, wherein the GlcNAc moiety is connected to the CBA through a α-N-glycosidic bond to give a Sd(A^{F})ₓ acceptor having the formula:

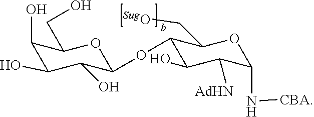

91. The method of statement 88, wherein the cell-binding agent is a protein, or comprises a protein portion, and wherein the GlcNAc moiety is conjugated to the cell-binding agent through an asparagine side chain via an α-N-glycosidic bond to give a Sd(A^{F})ₓ acceptor having the formula:

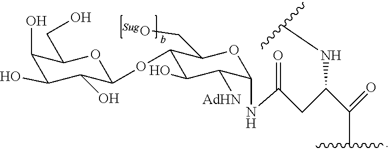

92. The method of any one of statements 88 to 91, wherein the nucleoside element of the nucleoside phosphate moiety is one of adenosine, guanosine, uridine, cytidine, or thymidine.

93. The method of any one of statements 88 to 92, wherein the nucleoside element of the nucleoside phosphate moiety is selected from the group consisting of: UDP, GDP, CDP, and CMP.

94. The method of any one of statements 88 to 93, wherein the compound of the formula Sd(A^{F})ₓ-P* has the formula:

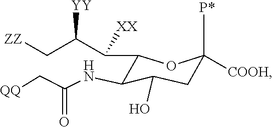

wherein:
P* is a nucleoside phosphate moiety; and
QQ is hydrogen or a functional group A;
ZZ is hydroxyl or a functional group A;
YY is hydroxyl or a functional group A; and/or
XX is hydroxyl or a functional group A;

and wherein at least one of QQ, ZZ, YY, and XX is a functional group A.

95. The method of any one of statements 88 to 94, wherein the compound of the formula Sd(A^{F})ₓ-P* has the formula:

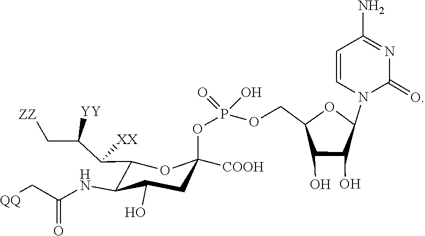

96. The method of any one of statements 88 to 95, wherein the Sd(A^{F})ₓ acceptor is provided by a process comprising the steps of:
a) providing a Gal acceptor having the formula:

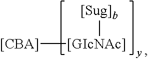

wherein CBA, GlcNAc, Sug, b, and y are defined as in statement 1;
and
b) contacting the Gal acceptor with a compound of the formula Gal-P* in the presence of a galactosyltransferase, wherein Gal is a Galactose moiety and P* is a nucleoside phosphate moiety;
optionally wherein,
c) the Gal acceptor is produced by contacting with a glycosidase a oligoglycosylated cell-binding agent having the formula:

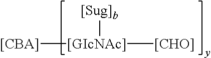

wherein CHO is a carbohydrate moiety.

97. The method of any one of statements 88 to 95, wherein the Sd(A^{F})ₓ acceptor has only one terminal galactose moiety.

98. The method of any one of statements 88 to 95, wherein the Sd(A^{F})ₓ acceptor has only two terminal galactose moieties.

99. The method of any one of statements 88 to 95, wherein the Sd(A^{F})ₓ acceptor has only three terminal galactose moieties.

100. The method of any one of statements 88 to 95, wherein the Sd(A^{F})ₓ acceptor has only four terminal galactose moieties.

101. The method of any one of statements 88 to 100, wherein the glycosyltransferase is a sialyltransferase.

102. The method of statement 101, wherein the sialyltransferase is α-(2,3)-sialyltransferase.

103. The method of statement 102, wherein the sialyltransferase is Pasteurella multocida α-(2,3)-sialyltransferase.

104. The method of statement 102, wherein the sialyltransferase is CMP-N-acetylneuraminate-β-galactosamide-α-2,3-sialyltransferase (ST3Gal IV).

105. The method of statement 101, wherein the sialyltransferase is α-(2,6)-sialyltransferase.

106. The method of statement 105, wherein the sialyltransferase is a β-galactoside α-(2,6)-sialyltransferase 1 (ST6Gal 1).

107. The method of statement 101, wherein the sialyltransferase is α-(2,8)-sialyltransferase.

108. The method of any one of statements 101, 102, 104, and 105 to 107, wherein the sialyltransferase is a mammalian sialyltransferase.

109. The method of statement 108, wherein the sialyltransferase is a human sialyltransferase.

110. The method of statement 108, wherein the sialyltransferase is a rat sialyltransferase.

111. The method of statement 101, wherein the sialyltransferase is a polypeptide having sialyltransferase activity and comprising a sequence having at least 70% sequence identity SEQ ID NO. 1, SEQ ID NO. 4, or SEQ ID NO. 7.

112. The method of statement 101, wherein the sialyltransferase has the sequence set out in SEQ ID NO. 1, SEQ ID NO. 4, or SEQ ID NO. 7.

113. The method of any one of statements 96 to 112, wherein the glycosidase is an endoglycosidase.

114. The method of any one of statements 96 to 113, wherein the glycosidase is in the class EC3.2.1.96.

115. The method of statement 113, wherein the endoglycosidase is endo-β-N-acetylglucosaminidase D, endo-β-N-acetylglucosaminidase H, endoglycosidase S, endo-β-N-acetylglucosaminidase M, endo-β-N-acetylglucosaminidase LL, endo-β-N-acetylglucosaminidase F1, endo-β-N-acetylglucosaminidase F2, or endo-β-N-acetylglucosaminidase F3.

116. The method of any one of statements 96 to 112, wherein the glycosidase is a combination of two or more endoglycosidases.

117. The method of any one of statements 96 to 112, wherein the glycosidase is a combination of two or more glycosidases in the class EC3.2.1.96.

118. The method of either one of statements 116 or 117, wherein the glycosidase is: endoglycosidase D and endoglycosidase S; endoglycosidase S and endoglycosidase LL; endoglycosidase D and endoglycosidase LL; endoglycosidase D and endoglycosidase H; endoglycosidase S and endoglycosidase H; endoglycosidase F1 and endoglycosidase F2; endoglycosidase F1 and endoglycosidase F3; endoglycosidase F2 and endoglycosidase F3; endoglycosidase D, endoglycosidase S and endoglycosidase LL; endoglycosidase D, endoglycosidase S and endoglycosidase H; or endoglycosidase D, endoglycosidase S and endoglycosidase F1.

119. The method of statement 113, wherein the endoglycosidase is Endo S as disclosed in Collin, M. and Olsén, A. (2001). The EMBO Journal. 20, 3046-3055.

120. The method of statement 113, wherein the endoglycosidase is a polypeptide having endoglycosidase activity and comprising a sequence having at least 70% sequence identity SEQ ID NO. 3, SEQ ID NO. 6, or SEQ ID NO. 9.

121. The method of statement 113, wherein the endoglycosidase has the sequence set out in SEQ ID NO. 3, SEQ ID NO. 6, or SEQ ID NO. 9.

122. The method of any one of statements 96 to 121, wherein the galactosyltransferase is human beta-1,4-galactosyltransferase 1 (B4GalT1).

123. The method of statement 122, wherein the galactosyltransferase is a polypeptide having galactosyltransferase activity and comprising a sequence having at least 70% sequence identity SEQ ID NO. 2, SEQ ID NO. 5, or SEQ ID NO. 8.

124. The method of statement 122, wherein the galactosyltransferase has the sequence set out in SEQ ID NO. 2, SEQ ID NO. 5, or SEQ ID NO. 8.

125. A glycoconjugate of any one of statements 1 to 52 for use in a method of treatment.

126. A glycoconjugate of any one of statements 1 to 52 for use in a method of treating a proliferative disorder.

127. A method of treating a proliferative disorder, the method comprising administering an effective amount of a glycoconjugate of any one of statements 1 to 52 to a subject.

128. Use of a glycoconjugate of any one of statements 1 to 52 in the manufacture of a medicament for the treatment of a proliferative disorder.

129. The glycoconjugate, method, or use of any one of statements 126 to 128, wherein the proliferative disorder is cancer.

130. The glycoconjugate, method, or use of statement 129, wherein the cancer is selected from the group consisting of: histocytoma, glioma, astrocyoma, neuroblastoma, osteoma, lung cancer, gastrointestinal cancer, bowel cancer, colon cancer, breast carinoma, ovarian carcinoma, prostate cancer, testicular cancer, liver cancer, kidney cancer, bladder cancer, pancreas cancer, renal cancer, brain cancer, sarcoma, liposarcoma, osteosarcoma, Kaposi's sarcoma, melanoma, lymphomas, myeloma, and leukemias.

131. A method for the preparation of a glycoconjugate, the method comprising the steps of:
a) providing an oligoglycosylated cell-binding agent having the formula:

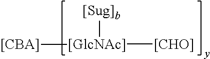

wherein CBA is a Cell Binding Agent;
GlcNAc is a N-acetyl-glucosamine moiety;
Sug is a sugar moiety, wherein b=1 or 0;
y=1 to 20; and
CHO is a carbohydrate moiety;
b) contacting the oligoglycosylated cell-binding agent with a glycosidase to produce a Gal acceptor having the formula:

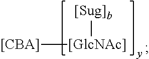

c) contacting the Gal acceptor with a compound of the formula Gal-P* in the presence of a galactosyltransferase to produce a Sd(A^{F})ₓ acceptor having the formula:

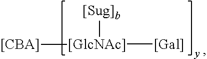

wherein Gal is a galactose moiety, and
P* is a nucleoside phosphate moiety;
d) contacting the Sd(A^{F})ₓ acceptor with a compound of the formula Sd(A^{F})ₓ-P* in the presence of a glycosyltransferase to produce a glycosylated cell-binding agent having the formula:

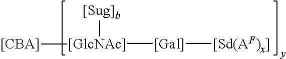

wherein:
   Sd(A^{F})ₓ is a sugar derivative comprising x functional groups A^{F}, wherein x=1, 2, 3, or 4;
e) reacting the glycosylated cell-binding agent with a compound of the formula payload-G^{L},
   wherein the payload is as according to any one of statements 39 to 52 and G^{L} is a linker group reactive with the functional group A^{F} of the glycosylated cell-binding agent,
to produce a glycoconjugate having the formula:

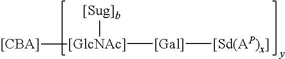

wherein:
   Sd(A^{P})ₓ is a sugar derivative comprising x conjugated payloads A^{P}, wherein x=1, 2, 3, or 4.

132. The method according to statement 131, wherein steps (b), (c), and (d) are performed in the same reaction volume.

133. The method according to either one of statements 131 or 32, wherein the Gal acceptor product of step (b) is not purified from the reaction volume before it is contacted with the galactosyltransferase of step (c).

134. The method according to any one of statements 131 to 133, wherein the Sd(A^{F})ₓ acceptor product of step (c) is not purified from the reaction volume before it is contacted with the glycosyltransferase of step (d).

135. The method according to any one of statements 131 to 134, wherein steps (b), (c), and (d) are performed at the same time.

136. The method according to any one of statements 131 to 135, wherein steps (b), (c), and (d) comprise an incubation of between 24 and 48 hours, such as about 36 hours.

137. The method according to statement 136, wherein the incubation is at about 37° C.

138. The method according to any one of statements 131 to 137, wherein the method further comprises an additional step (d′) between steps (d) and (e), wherein step (d′) comprises the addition of further Sd(A^{F})ₓ-P* and/or glycosyltransferase.

139. The method according to statement 138, wherein the further Sd(A^{F})ₓ-P* and/or glycosyltransferase are added to the products of step (d).

140. The method according to either one of statements 138 or 139, wherein the further Sd(A^{F})ₓ-P* and/or glycosyltransferase are added on completion of the incubation of step (d).

141. The method according to any one of statements 138 to 140, wherein step (d′) comprises an incubation of between 24 and 48 hours, such as about 36 hours.

142. The method according to statement 141, wherein the incubation is at about 37° C.

### EXAMPLES

### Example 1: Synthesis of Drug-Linker Payload, PL1603

The PL1603 drug-linker was synthesised for use as a payload suitable for conjugation to the glycosylated antibody intermediates described herein.

Synthesis Method

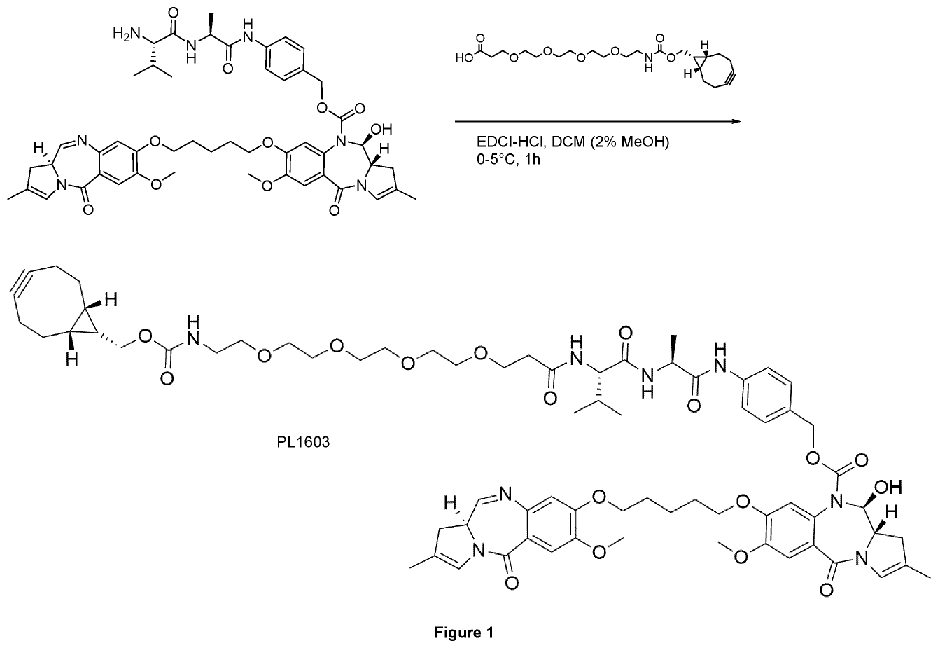

See FIG. 1.

SG3305 (600 mg, 1.0 eq), Endo-BCN-PGE4-acid (1.2 eq) and EDCI-HCl (1.2 eq) were taken up in DCM (15 vol, 2% MeOH) and stirred at 0-5° C. (The synthesis of Endo-BCN-PEG4 is described in, for example, WO2016/053107 at page 142. Endo-BCN-PEG4 is also commercially available from, for example, Broadpharm®. The synthesis of SG3305 is described in, for example, Tiberghein et al., ACS Med Chem Lett 2016 7(11) 983-987 [DOI: 10.1021/acsmedchemlett.6b00062])

Upon completion of reaction, the reaction was quenched with purified water (10 vol). The mixture was partitioned and the organic layer was washed with brine, dried over sodium sulphate and concentrated under reduced pressure to give crude PL1603 (650 mg, 93.2%, 73.57% HPLC purity).

Crude PL1603 (400 mg) was purified by RP-HPLC (C18, MeCN:H₂O) and product containing fractions were combined and lyophilised to give PL1603 as a white solid (130 mg, 33%, 94.61% HPLC purity).

### Example 2: Antibody Remodelling and Conjugation, Approach 1

Antibody Remodelling

The N-linked oligosaccharides on the Herceptin antibody were remodelled according to the methods described in Li et al. 2014 (Angew Chem Int Ed Engl., 2014, Jul. 7; 53(28):7179-82).

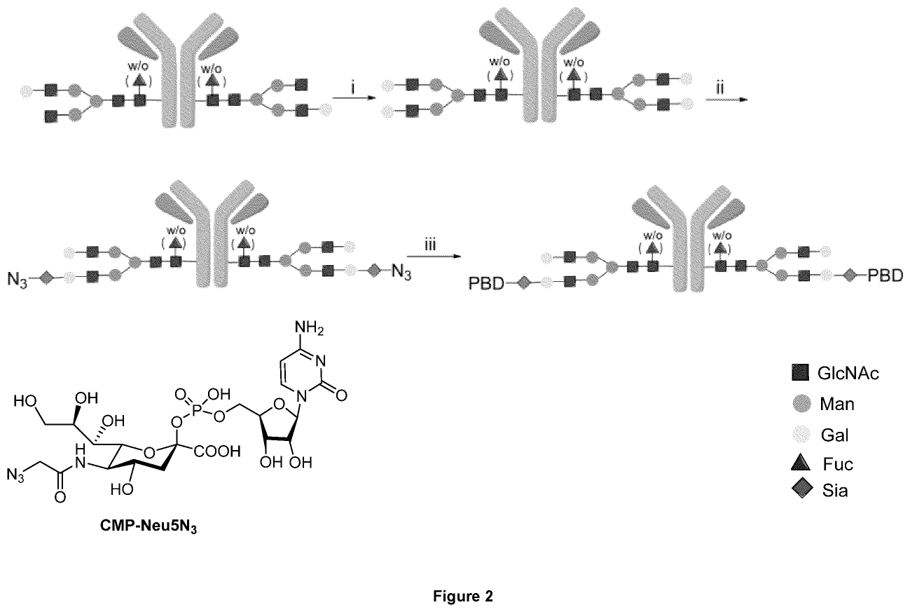

See FIG. 2, steps (i) and (ii).

Conjugation

9.2 mg/ml of the remodelled Herceptin in 50 mM Cacodylate buffer pH 7.6 was conjugated by the addition of 20 molar equivalents of PL1603 (10 mM stock in DMA, structure provided in FIG. 1) and DMA to a final Co solvent level of 20% v/v. The conjugation reaction was incubated in a 20° C. water bath for 66 hours. A DAR by PLRP of 1.4 was achieved (batch SON160-16).

The resulting glycoconjugate is herein termed ‘Her-PL1603-App1’.

See FIG. 2, step (iii).

### Example 3: Antibody Remodelling and Conjugation, Approach 2

Rationale for New Approach

In Approach 1, the activity of the recombinant sialyltransferase ST6Gal1 to the α(1,3)- and α(1,6)-arm of the biantennary N-glycan of the Fc region of antibodies can be differential by controlling the ratio of CMP-sialic acid and antibody. This can result in ADCs having DAR2 or DAR4. However, the careful controlling of the reaction stoichiometry that is required impacts on product reproducibility between batches.

Accordingly, alternative oligosaccharide structures were investigated with the aim of identifying oligosaccharide structures that were both obtainable using available synthetic methods and offered advantageous glycoconjugate properties.

A key discovery was the unexpected ability of wild-type human β4GalT1 galactosyl-transferase to transfer a galactose residue onto a α1-6 fucosylated GlcNAc residue. This reaction does not occur in nature. Moreover, it was found that the resulting galactosylated oligosaccharide could be further modified with the addition of an azido-modified sialic acid by the ST6Gal1 sialyltransferase.

Antibody Remodelling

The N-linked oligosaccharides on the Herceptin antibody were remodelled according to the following method:

Endo S. Treatment.

Trimming of IgG glycan was undergone using Endo S cloned from Streptococcus pyogenes and overexpressed as a fusion to the chitin binding domain in E. coli. (New England BioLabs). To the IgG antibody (10 mg/mL) in 30 mM histidine, 200 mM sorbitol and 0.02% tween-20, Endo S (0.13 mL, 100 kU/mL) in 10 mM Tris, 25 mM NaCl, 2.5 mM EDTA, 2.5 mM CaCl2, 25 mM sodium acetate was added. The resulting solution was incubated for approximately 48 hours at 37° C. followed by Protein A Sepharose Column (GE Healthcare) purification, buffer exchanging and concentrated into 1.2 mL of 50 mM MOPS containing 20 mM MnCl₂.

Galactosylation of the IgG

Galactosylation of IgG bearing truncated N-glycan was achieved by addition of β-1,4-galactosyl transferase (200 μg/mL) to the Endo S treatment resulting material in 50 mM MOPS, 20 mM MnCl2, 10 mM UDP-galactose, pH 7.2, 80 μg/mL BSA, 85 U/mL calf intestine alkaline phosphatase and incubation at 37° C. for 70 h. To ensure complete galactosylation, an additional aliquot of UDP-galactose and galactosyl transferase were added to the reaction and incubated at 37° C. for an additional 24 h. The galactosylated IgG was purified using a Protein A Sepharose Column and the solution was exchanged in 50 mM cacodylate, pH 7.6 using an Amicon 10 kDa cutoff spin concentrator (Millipore).

Synthesis of CMP-Neu5N3 and CMP-Neu9N3

Sialic acid aldolase (0.2 U/μL, 5 μL), and CMP-sialic acid synthetase (0.2 U/μL, 5 μL) were added to a mixture of N-azidoacetyl-D-mannosamine (5 mg, 0.019 mmol) in tris-HCl buffer (100 mM, pH 8.9, 20 mM MgCl₂, 1.9 mL), containing sodium pyruvate (10.5 mg, 0.095 mmol) and CTP (10 mg, 0.019 mmol). The tube was incubated at 37° C., and progress of the reaction was monitored by TLC (EtOH:aq. NH₄HCO₃ (1 M) 7:3, v:v), which after 5 hour indicated completion of the reaction. EtOH (3 mL) was added, and the precipitate was removed by centrifugation and the supernatant was concentrated under reduced pressure. The residue was redissolved in distilled water (500 μL) followed by lyophilization to provide a crude material that was applied to a Biogel fine P-2 column (50*1 cm, eluted with 0.1 M NH₄HCO₃ at 4° C. in dark). The product was detected by TLC, and appropriate fractions were combined and lyophilized to provide CMP-Neu5N₃ as an amorphous white solid (10.1 mg, 81%).

¹H NMR (300 MHz, d₂o) δ 7.82 (d, J=7.5 Hz, 1H, H-6, cyt), 5.97 (d, J=7.6 Hz, 1H, H-5, cyt), 5.84 (d, J=4.2 Hz, 1H, H-1, rib), 4.18 (dd, J=7.4, 4.4 Hz, 2H, H-2+H-3, rib), 4.14-4.04 (m, 4H, H-4+H-5 rib, H-6 Neu), 4.04-3.97 (m, 1H, H-4), 3.95 (s, 2H, N₃CH₂CO), 3.87 (t, J=10.2 Hz, 1H, H-5), 3.82-3.74 (m, 1H, H-8), 3.72 (m, 1H, H-9a), 3.49 (d, J=11.8 Hz, 1H, H-9b), 3.30 (d, J=9.5 Hz, 1H, H-7), 2.36 (dd, J=13.3, 4.6 Hz, 1H, H-3 eq), 1.51 (td, J=12.0, 5.6 Hz, 1H, H-3ax). HRMS (ESI): m/z calcd for C₂₀H₃₀N₇O₁₆P [M−H]⁻: 654.1414; found: 653.9477.

CMP-Neu9N₃ was prepared following the reported procedure. CTP (126 mg, 0.24 mmol) was added to a solution of 5-Acetamido-9-azido-3,5,9-tri-deoxy-D-glycero-D-galacto-2-nonulosonic acid (50 mg, 0.15 mmol) in a Tris-HCl buffer (0.1 M, 9 mL, pH 8.9) containing MgCl₂ (20 mM). The recombinant CMP-sialic acid synthetase from N. meningitis (4.0 U) and the inorganic pyrophosphatase from S. cerevisiae (2.0 U) were added and the reaction mixture was incubated at 37° C. with shaking. The progress of the reaction was monitored by TLC (isopropanol: 20 mM NH₄OH, 4:1, v:v), which after 3 h indicated completion of the reaction. Ethanol (80 mL) was added and the mixture was kept on ice for 2 h prior to centrifugation. The supernatant was decanted and the pellet (mostly inorganic salts) was re-suspended in EtOH (30 mL), cooled on ice for 1 h and centrifuged. The combined ethanol extracts were concentrated in vacuo providing crude material (168 mg). Ethanol (1.8 mL) was slowly added to the material dissolved in H₂O (0.2 mL) and precipitation occurred immediately. The mixture was kept on ice for 2 h.

Next, the supernatant was removed after centrifugation and the white pellet was dried and purified on a column of extra-fine Biogel P-2 eluted with 0.1 M NH₄HCO₃ at 4° C. The appropriate fractions were detected by UV and TLC (as above), collected, concentrated in vacuo (bath temperature <25° C.) and lyophilized to afford CMP-Neu9N3 (60 mg, 62%). ¹H NMR (D₂O containing 0.1 M NH₄HCO₃, 600 MHz): δ 7.82 (d, 1H, J_{5,6}=7.8 Hz, H-6, cyt), 5.97 (d, 1H, J_{5,6}=7.8 Hz, H-5, cyt), 5.82 (d, 1H, J_{1,2}=4.8 Hz, H-1 rib), 4.17 (t, 1H, J=4.8), 4.13 (t, 1H, J=4.8 Hz), 4.08 (m, 3H), 3.99 (d, 1H, J=12.0 Hz), 3.90 (m, 2H), 3.78 (t, 1H), 3.49 (dd, 1H, J=2.4, 13.2 Hz, H-9a), 3.35 (dd, 1H, J=6.0, 13.2 Hz, H-9b), 3.31 (dd, 1H, J=9.6 Hz), 2.33 (dd, 1H, J_{3eq,4}=4.8 Hz, J_{3eq,3ax}=13.2 Hz, H-3 eq), 1.90 (s, 3H, Me), 1.55 (ddd, 1H, J_{3ax,P}=6.0 Hz, J_{3ax,3eq}=13.2 Hz, J_{3ax,4}=12.0 Hz, H-3ax); ¹³C NMR (D₂O, containing 0.1 M NH₄HCO₃, 600 MHz): δ 174.2, 170.4, 166.0, 160.7, 141.4, 96.5, 88.8, 82.9, 74.2, 71.5, 69.3, 69.1, 67.4, 64.9, 53.0, 51.7, 41.0, 22.0; ESI-MS: calcd for C₂₀H₂₈N₇O₁₅P²⁻ [M+H]⁻: m/z: 638.1470; found 638.1421.

Sialylation of the IgG

The sialylation of galactosylated IgG was performed in 50 mM cacodylate, 14 mg/mL of IgG, 8 mM CMP-Neu5N₃, 90 μg/ml BSA, 90 U/mL calf intestine alkaline phosphatase and 0.4 mg/mL GFP-ST6Gal I at pH 7.6 and incubated at 37° C. for 4 days followed by Protein A Sepharose Column purification and buffer exchanging to 50 mM cacodylate. The extent of sialylation was monitored by LC-MS as described previously using a Shimadzu LCMS-IT-TOF Mass Spectrometer. Following every 48 hours incubation, the sample was buffer exchanged with 50 mM cacodylate, pH7.6 using an Amicon 10 kDa cutoff spin concentrator to remove CMP, an inhibitor of ST6Gal I and an additional aliquot of CMP-Neu5N₃ and α2-6 sialyltransferase were added back to this washed preparation.

Conjugation

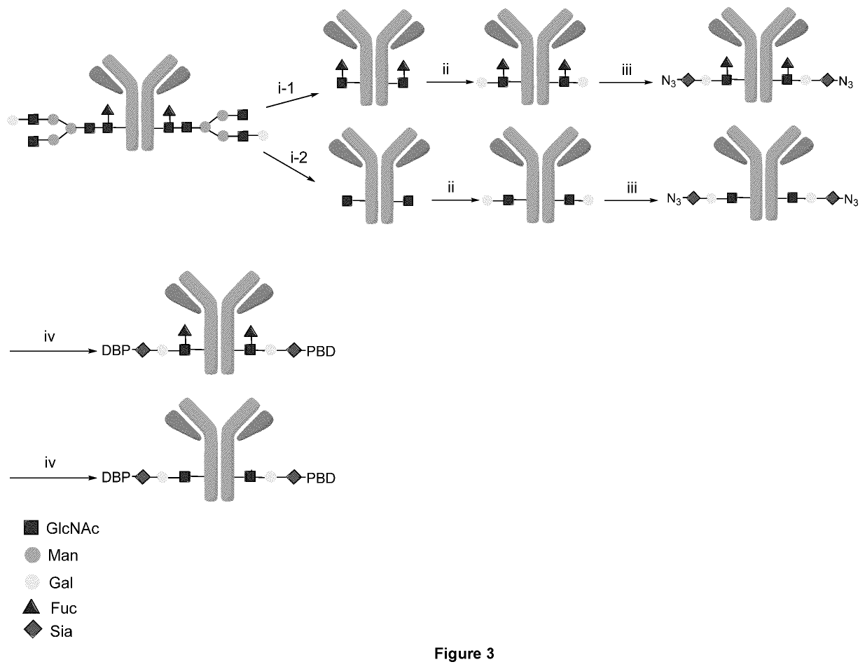

10 mg/ml of the remodelled Herceptin in 50 mM Cacodylate buffer pH 7.6 was conjugated by the addition of 7.5 molar equivalents of PL1603 (10 mM stock in DMA, structure provided in FIG. 3) and DMA to a final Co solvent level of 20% v/v. The conjugation reaction was incubated in a 20° C. water bath overnight. A DAR of 1.8 was achieved (batch SON180-19) with purification by PLRP.

The conjugation reaction in approach 2 is notable for being considerably faster than the corresponding approach 1. This goes against initial expectations, which were that the termini of the longer glycans would be more accessible and so easier to modify. In fact, the data shows that the shorter glycans of approach 2 were more readily modifiable.

The resulting glycoconjugate is herein termed ‘Her-PL1603-App2’.

See FIG. 3, step (iv).

### Example 4: Glycoconjugate Properties

Physical Properties

Her-PL1603-App1 and Her-PL1603-App2 were analysed by hydrophobic Interaction Chromatography. This was carried out using column a MabPac HIC-Butyl, 5 μm, 4.6×100 mm column (Thermo, #882558, lot 01425138, serial nb 001303) with a MabPac HIC-Butyl, 5 μm, 4.6×10 mm Guard cartridge: (Thermo, #882559, lot 1425011). With a Mobile Phase A of 1.5 M (NH₄)₂SO₄, 25 mM NaPO₄ (pH 7.4) and a Mobile Phase B of 80% 25 mM NaPO₄ (pH 7.4), 20% CH₃CN. The assay was run at 0.8 ml per minute and a column temperature of 25° C. A 10 μl sample load at 1 mg/ml was used for the analysis.

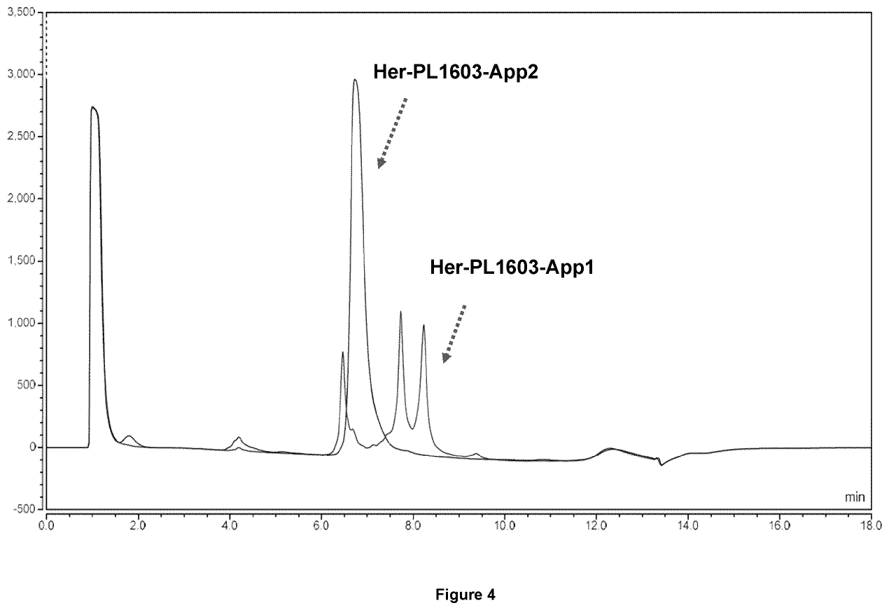

The HIC showed a distinct difference between the two ADCs, with Her-PL1603-App1 separating into multiple hydrophobic species, whilst Her-PL1603-App2 eluted as one more hydrophilic peak (see FIG. 4).

The increased hydrophilicity of Her-PL1603-App2 over Her-PL1603-App1 is prima facie surprising in view of the fact that Her-PL1603-App2 has considerably fewer sugar residues than Her-PL1603-App1 (compare FIGS. 2 and 3).

Sugar residues are very hydrophilic moieties, so the expectation is that increasing their number would increase overall molecule hydrophilicity. The fact that this is not the case here suggests a more complex interaction is occurring between the antibody, oligosaccharide, and drug-linker elements of the ADC.

In-Vitro Binding to Her2

Her2 is the cognate antigen of the Herceptin antibody. Binding of Her-PL1603-App1 and Her-PL1603-App2 was determined by ELISA. Maxisorp ELISA plates were coated with 0.5 μg/mL recombinant human Her2 at room temperature, before blocking with 3% BSA. Sample titrations were prepared in assay buffer (0.1% BSA/0.05% tween) between 66.6 and 0.016 nM in quartering dilutions. Samples were then incubated on the antigen coated plate for 1 hour. A mouse anti-human antibody conjugated to HRP was used for detection (Sanquin M1328) and incubated for 1 hour before washing and adding the detection agent, TMB for 10 minutes before stopping the reaction with HCl. Binding absorbance data was acquired on the Spectramax plate reader at 450 nm.

For comparison, Her2 binding was also assessed for ‘Her-C220’ [an unconjugated version of Herceptin in which 3 of the 4 interchain cysteines have been substituted for either V (in the heavy chain) or S (in the light chain)] and B12 [an unconjugated monoclonal antibody against the HIV-1 protein; used here as a control].

| ADC/antibody | IC50 (nM) |
|---|---|
| Her-PL1603-App1 | 0.23 |
| Her-PL1603-App2 | 0.38 |
| Her2-C220 | 0.17 |
| B12 | No Binding |

The two ADCs bound to Her2 with similar affinity.

In-Vitro Cytotoxicity

The in vitro cytotoxicity of Her-PL1603-App1 and Her-PL1603-App2 against Her2+ve N87 cells was determined. A “thaw and go” cytotox assay was used to determine the cytoxicity, N87 cells were taken from cryogenic storage and seeded to 5×10⁴ cells/mL (5×10³ cells/well) on an EDGE plate then incubated for a minimum of 2 hours at 37° C./5% CO₂/absolute humidity. An 11 point, 1 in 4 serial titration of the test and control samples was prepared in duplicate from 500 nM to 0.4768 pM with a final negative control. The titrated samples were added to the EDGE plate containing cells and incubated for 5 days at 37° C./5% CO₂/absolute humidity. Celltiter Aqueous One solution was added and the plate was incubated at 37° C./5% CO₂/absolute humidity for a final time, before measuring absorbance at 490 nm using the SpectraMax plate reader.

For comparison, cytotoxicity was also assessed for ‘Her2×ADC’ [Her2-C220 conjugated to the PBD drug-linker SG3249 (Tesirine) at the C220 residue] and B12-C220-SG3249 [the B12 antibody conjugated to the PBD drug-linker SG3249 (Tesirine) at the C220 residue].

| ADC | IC50 (pM) |
|---|---|
| Her-PL1603-App1 | 24.7 |
| Her-PL1603-App2 | 21.7 |
| Her2xADC | 17.9 |
| B12-C220-SG3249 | 2994 |

Her-PL1603-App1 and Her-PL1603-App2 were found to have similar cytotoxicity to each other and also to the benchmark Her2×ADC. Significantly less cell kill was observed with the non-Her2-binding B12 control ADC.

In-Vivo Efficacy

The in vivo efficacy of the Her-PL1603-App1 and Her-PL1603-App2 conjugates was measured in the breast cancer Her2+ve BT474 xenograft model. For comparison, in vivo efficacy was also assessed for ‘Her2×ADC’.

Female severe combined immunodeficient mice (Fox Chase SCID®, CB17/lcr-Prkdcscid/lcrlcoCrl, Charles River) were ten weeks old with a body weight (BW) range of 16.1 to 21.8 g on Day 1 of the study. On the day of tumor implant, each test mouse received a 1 mm³ BT474 fragment implanted subcutaneously in the right flank, and tumor growth was monitored as the average size approached the target range of 100 to 150 mm³. Tumors were measured in two dimensions using calipers, and volume was calculated using the formula:

Tumor Volume (mm3)=w²×l/2

where w=width and l=length, in mm, of the tumor. Tumor weight may be estimated with the assumption that 1 mg is equivalent to 1 mm³ of tumor volume.

Thirty-six days after tumor implantation, designated as Day 1 of the study, the animals were sorted into groups each consisting of ten mice with individual tumor volumes of 75 to 172 mm³ and group mean tumor volumes of 119-121 mm³. On Day 1 of the study, drugs were administered intravenously (i.v.) in a single injection (qd×1) via tail vein injection. The dosing volume was 0.2 mL per 20 grams of body weight (10 mL/kg), and was scaled to the body weight of each individual animal. Tumors were measured using calipers twice per week, and each animal was euthanized when its tumor reached the endpoint volume of 1000 mm3 or at the end of the study (Day 59), whichever came first.

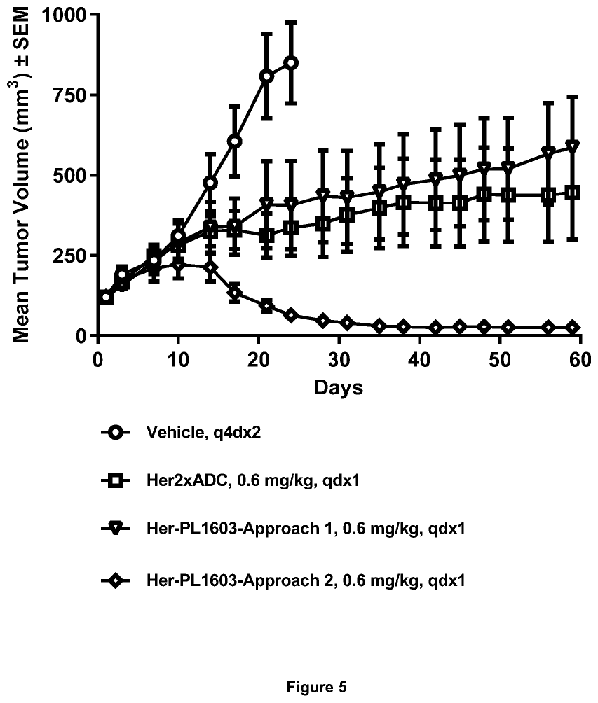

Results are shown in FIG. 5.

The minimal efficacious dose (MED) of Her2×ADC and Her-PL1603-App1 was >0.6 mg/kg, while the MED for Her-PL1603-App2 was determined to be 0.3 mg/kg.

Tolerability: Rat Toxicology Study

Her2×ADC, Her-PL1603-App1 and Her-PL1603-App2 were evaluated in a single intravenous dose rat tolerability study.

Male sprague-dawley rats (n=3/group) were dosed at 4 mg/kg with Her-PL1603-App1 or 2 & 4 mg/kg with Her-PL1603-App2 on day 1, with necropsy on day 21 following dosing. Bodyweights and food consumption were monitored frequently with in-life sampling for clinical pathology (blood on days 8 and 21) and repeated sampling for pharmacokinetics. At necropsy, macroscopic observations were taken with selected organs weighed and retained for possible histopathology.

Her2×ADC was evaluated at 1.5 mg/kg, single intravenous injection to male Sprague Dawley rats was associated with reduced overall body weight gain (overall bodyweight gain was 39% lower), associated with reduced food consumption. White blood cell numbers were reduced on day 8 (−61%), with evidence of recovery by day 21. At necropsy, reduced thymus, spleen, testes and prostate/seminal vesicle weights and increased adrenal gland weight were observed.

Her-PL1603-App1 was poorly tolerated at 4 mg/kg, resulting in early euthanasia 11 days after dosing in 2 out of 3 animals. Bodyweight gain was markedly reduced in these animals, with none of the expected weight gain after dosing. Several haematology parameters were markedly reduced on day 8 (reticulocytes (−93%), white blood cells (−86%) and platelets (−66%)), with no evidence of recovery.

Her-PL1603-App2 was clinically well tolerated at 2 & 4 mg/kg. Bodyweight gain was dose-dependently reduced (overall bodyweight gain was 55% lower at 4 mg/kg), consistent with reduced food consumption. Several haematology parameters were reduced on day 8 (reticulocytes (−52%), white blood cells (−68%) and platelets (−22%)), with evidence of recovery by day 21. At necropsy, dose-dependent reductions in thymus, liver and spleen weights and increased lung weights were noted, with two animals presenting with pale kidneys at 4 mg/kg.

The maximum tolerated dose (MTD) for Her2×ADC was 1.5 mg/kg (the highest dose tested).

The maximum tolerated dose (MTD) for Her-PL1603-App1 was lower than 4 mg/kg.

The maximum tolerated dose (MTD) for Her-PL1603-App2 was 4 mg/kg.

Therapeutic Index

The Therapeutic Index (TI) of the ADCs may be calculated by first determining the Human equivalent dose of the MED and MTD and then dividing the HED of the MTD by the HED of the MED, as shown below:

| | mg/kg | | | | |
|---|---|---|---|---|---|
| | | MTD | HED of | HED of | |
| | MED | Rat | MED | MTD rat | TI |
| Her2xADC | >0.6 | 1.5 | >0.050 | 0.22 | <4 |
| Her-PL1603-App1 | >0.6 | <4.0 | >0.050 | <0.60 | <<12 |
| Her-PL1603-App2 | 0.3 | 4.0 | 0.025 | 0.60 | 24 |

MED: Minimal effective dose in mice
HED: Human equivalent dose
MTD: Maximal Tolerable dose
TI: Therapeutic Index

Her-PL1603-App2 exhibits a Therapeutic Index of at least twice that of Her-PL1603-App1.

Her-PL1603-App2 exhibits a Therapeutic Index of about 6 times that of Her2×ADC.

Pharmacokinetics (PK) of Her-PL1603-App1 and Her-PL1603-App2 in Rats

Plasma samples of rats dosed with a single dose of 2 and or 4 mg/kg of Her-PL1603-App1 and Her-PL1603-App2 and samples were taken 1, 3, 6, 48, 72, 168, 336 and 480 h after dosing. The samples were analysed for total human IgG and PBD conjugated IgG as described in Zammarchi Blood vol 131 (10), 1094-1105 2018.

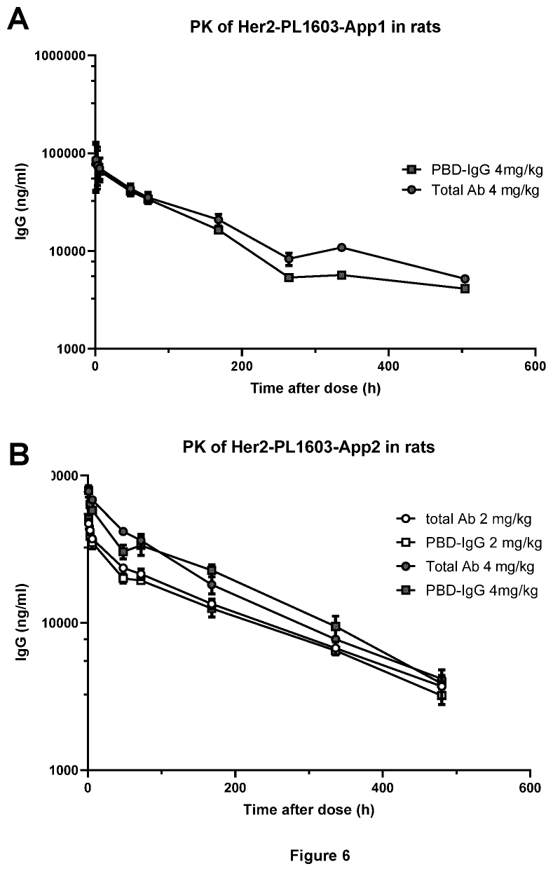

FIG. 6A shows comparable PK profiles of Her2-PL1603-App1 for total IgG and PBD-IgG at 4 mg, which shows the conjugate is highly stable.

FIG. 6B shows comparable PK profiles of Her2-PL1603-App2 for total IgG and PBD-IgG both at 2 and 4 mg, which shows the conjugate is highly stable.

Further Comments on Properties

A further advantage of ‘Approach 2’ as described above in Examples 1-4 is that it is easier to control the DAR at 2. In earlier approaches employing an intact glycan, it was more difficult to control the DAR at 2, necessitating careful control of reaction conditions.

In addition, Approach 2 abolishes Fc(gamma) receptor activity which is an advantage for a number of ADC applications.

### Example 5: One-Pot Remodelling

The enzymes used were produced in CHO cell as recombinant proteins at Evitria (https://www.evitria.com) and purified in-house at ADC-Therapeutics (Sequences shown below as SEQ ID NO. 4 (ST6Gal1), SEQ ID NO. 5 (Beta4Gal), and SEQ ID NO. 6 (EndoS)). Other reagents were:
UDP-Galactose (Merck Life Sciences UK Limited. Cat: U4500-25MG)
CMP-Sialic acid (Merck Life Sciences UK Limited Cat: 5052230001)
CIAP (Calf Intestinal Alkaline Phosphatase) (ThermoFisher Cat: 18009019)

Initial experiments focused on a one pot reaction, ensuring that the Endo S and Beta 4GAL (β-1,4-galactosyl transferase) resulted in full galactosylation. To this end three aliquots were set-up under the following conditions:
1. 2 mls of antibody, buffer swapped in 50 Kd Mw Amicon into 50 mM MOPS/20 mM MgCl2 and adjusted to 10 mg/ml (˜1.8 mls)
2. STGA6 & Beta-Gal buffer swapped into 50 mM MOPS/20 mM MgCl2 and quantified by OD280
3. 3×500 ul Aliquots of the Ab created (5 mgs of Ab in total)
4. To all Aliquots 25 ug of Endo S added (0.5%)
5. To all Aliquots 5 ul of CIAP per 1 mL Ab @10 mg/mL (ie 2.5 uL)
6. To all Aliquots add 50 ul of 300 mM UDP-Galactose=10 mM (dissolved in 150 ul MgCl2/MOPS buffer) per 1 mL Ab @10 mg/mL added (50 ul/ml) (Merck Life Sciences UK Limited. Cat: U4500-25MG)
7. To all Aliquots beta4Gal was added to 2.5% (125 ug/5 mg of Ab)
8. To Aliquots 2&3 add a further 2.5 ul CIAP
9. Add to aliquot 2&3 add CMP-Sialic acid to 4 mM (from 25 mM solution) ie 80 ul in 500 ul (Merck Life Sciences UK Limited Cat: 5052230001)
10. Add to aliquot 2&3 ST6GAL1 to 5% ie 250 ugs in 500 ul
11. Samples incubated for 36 hours at 37° C.
12. A further 5 ul CIP Aliquot, 50 ul UDP-Galactose (dissolve in 150 ul of MOPS/MgCl2) & 33 ul Beta4 Gal added to Aliquot 3
13. All aliquots incubated for a further 24 hrs
14. Samples purified up using Protein A spin columns (Thermo) and quantified by OD280.

The three aliquots were analysed by LCMS and showed that the Aliquot 1, Endo S and Beta4Gal treated, had 100% galactosylation, Aliquot 2, Endo S/Beta4Gal/ST6Gal1, showed 92% Galactosylation with 34% sialylation and Aliquot 3, Endo S/Beta4Gal/ST6Gal1 plus additional Beta4Gal/UDP-Galactose showing 100% galactosylation and 35% sialylation.

The data indicated that additional Beta4Gal/UDP-Galactose addition was not needed and that the first two steps of the process could be a one-pot reaction.

The degree of sialylation of 35% was not considered sufficient, and potentially caused by the “poisoning” of the reaction by CMP. To attempt to overcome this a second set of reactions were set-up where a similar protocol was followed, except after 36 hours, one sample had additional ST6Gal1 and CMP-Sialic added and left for an additional for 36 hours at 37° C. The control sample Endo S/Beta4Gal, was incubated for the same length of time as the Endo S/Beta4Gal/ST6Gal1+A ST6Gal1/CMP-Sialic acid sample. The samples were again purified via Protein A chromatography and analysed by LCMS.

As before the, the one pot reaction of Endo S and Beta4Gal resulted in 100% galactosylation, the reaction with the additional ST6Gal1 and CMP-Sialic acid showed 88% sialylation, compared to the previous 35% incorporation, showing that potential “poisoning” by the CMP can be overcome by the addition of additional ST6Gal1 and CMP-Sialic acid and that the process is able to be run as a one-pot process without additional clean-up between stages.

SEQUENCE LISTING PART OF THE DESCRIPTION

SEQ ID NO. 1:
MIHTNLKKKFSCCVLVFLLFAVICVWKEKKKGSYYDSFKLQTKEFQVLKSLGKLAMGSDSQS
VSSSSTQDPHRGRQTLGSLRGLAKAKPEASFQVWNKDSSSKNLIPRLQKIWKNYLSMNKY
KVSYKGPGPGIKFSAEALRCHLRDHVNVSMVEVTDFPFNTSEWEGYLPKESIRTKAGPWG
RCAVVSSAGSLKSSQLGREIDDHDAVLRFNGAPTANFQQDVGTKTTIRLMNSQLVTTEKRF
LKDSLYNEGILIVWDPSVYHSDIPKWYQNPDYNFFNNYKTYRKLHPNQPFYILKPQMPWEL
WDILQEISPEEIQPNPPSSGMLGIIIMMTLCDQVDIYEFLPSKRKTDVCYYYQKFFDSACTMG
AYHPLLYEKNLVKHLNQGTDEDIYLLGKATLPGFRTIHC
SEQ ID NO. 2:
MRLREPLLSGSAAMPGASLQRACRLLVAVCALHLGVTLVYYLAGRDLSRLPQLVGVSTPLQ
GGSNSAAAIGQSSGELRTGGARPPPPLGASSQPRPGGDSSPVVDSGPGPASNLTSVPVP
HTTALSLPACPEESPLLVGPMLIEFNMPVDLELVAKQNPNVKMGGRYAPRDCVSPHKVAIII
PFRNRQEHLKYWLYYLHPVLQRQQLDYGIYVINQAGDTIFNRAKLLNVGFQEALKDYDYTC
FVFSDVDLIPMNDHNAYRCFSQPRHISVAMDKFGFSLPYVQYFGGVSALSKQQFLTINGFP
NNYWGWGGEDDDIFNRLVFRGMSISRPNAVVGRCRMIRHSRDKKNEPNPQRFDRIAHTKE
TMLSDGLNSLTYQVLDVQRYPLYTQITVDIGTPS
SEQ ID NO. 3:
MDKHLLVKRTLGCVCAATLMGAALATHHDSLNTVKAEEKTVQVQKGLPSIDSLHYLSENSK
KEFKEELSKAGQESQKVKEILAKAQQADKQAQELAKMKIPEKIPMKPLHGPLYGGYFRTWH
DKTSDPTEKDKVNSMGELPKEVDLAFIFHDWTKDYSLFWKELATKHVPKLNKQGTRVIRTIP
WRFLAGGDNSGIAEDTSKYPNTPEGNKALAKAIVDEYVYKYNLDGLDVDVEHDSIPKVDKK
EDTAGVERSIQVFEEIGKLIGPKGVDKSRLFIMDSTYMADKNPLIERGAPYINLLLVQVYGSQ
GEKGGWEPVSNRPEKTMEERWQGYSKYIRPEQYMIGFSFYEENAQEGNLWYDINSRKDE
DKANGINTDITGTRAERYARWQPKTGGVKGGIFSYAIDRDGVAHQPKKYAKQKEFKDATDN
IFHSDYSVSKALKTVMLKDKSYDLIDEKDFPDKALREAVMAQVGTRKGDLERFNGTLRLDN
PAIQSLEGLNKFKKLAQLDLIGLSRITKLDRSVLPANMKPGKDTLETVLETYKKDNKEEPATIP
PVSLKVSGLTGLKELDLSGFDRETLAGLDAATLTSLEKVDISGNKLDLAPGTENRQIFDTMLS
TISNHVGSNEQTVKFDKQKPTGHYPDTYGKTSLRLPVANEKVDLQSQLLFGTVTNQGTLIN
SEADYKAYQNHKIAGRSFVDSNYHYNNFKVSYENYTVKVTDSTLGTTTDKTLATDKEETYK
VDFFSPADKTKAVHTAKVIVGDEKTMMVNLAEGATVIGGSADPVNARKVFDGQLGSETDNI
SLGWDSKQSIIFKLKEDGLIKHWRFFNDSARNPETTNKPIQEASLQIFNIKDYNLDNLLENPN
KFDDEKYWITVDTYSAQGERATAFSNTLNNITSKYWRVVFDTKGDRYSSPVVPELQILGYPL
PNADTIMKTVTTAKELSQQKDKFSQKMLDELKIKEMALETSLNSKIFDVTAINANAGVLKDCI
EKRQLLKK
SEQ ID NO. 4:
HHHHHHWSHPQFEKGGGSGGGSGGSSAWSHPQFEKFQVLKSLGKLAMGSDSQSVSSSS
TQDPHRGRQTLGSLRGLAKAKPEASFQVWNKDSSSKNLIPRLQKIWKNYLSMNKYKVSYK
GPGPGIKFSAEALRCHLRDHVNVSMVEVTDFPFNTSEWEGYLPKESIRTKAGPWGRCAVV
SSAGSLKSSQLGREIDDHDAVLRFNGAPTANFQQDVGTKTTIRLMNSQLVTTEKRFLKDSL
YNEGILIVWDPSVYHSDIPKWYQNPDYNFFNNYKTYRKLHPNQPFYILKPQMPWELWDILQ
EISPEEIQPNPPSSGMLGIIIMMTLCDQVDIYEFLPSKRKTDVCYYYQKFFDSACTMGAYHPL
LYEKNLVKHLNQGTDEDIYLLGKATLPGFRTIHC
SEQ ID NO. 5:
HHHHHHHHHHRDLSRLPQLVGVSTPLQGGSNSAAAIGQSSGELRTGGARPPPPLGASSQ
PRPGGDSSPVVDSGPGPASNLTSVPVPHTTALSLPACPEESPLLVGPMLIEFNMPVDLELV
AKQNPNVKMGGRYAPRDCVSPHKVAIIIPFRNRQEHLKYWLYYLHPVLQRQQLDYGIYVIN
QAGDTIFNRAKLLNVGFQEALKDYDYTCFVFSDVDLIPMNDHNAYRCFSQPRHISVAMDKF
GFSLPYVQYFGGVSALSKQQFLTINGFPNNYWGWGGEDDDIFNRLVFRGMSISRPNAVVG
RCRMIRHSRDKKNEPNPQRFDRIAHTKETMLSDGLNSLTYQVLDVQRYPLYTQITVDIGTPS
SEQ ID NO. 6:
EEKTVQVQKGLPSIDSLHYLSENSKKEFKEELSKAGQESQKVKEILAKAQQADKQAQELAK
MKIPEKIPMKPLHGPLYGGYFRTWHDKTSDPTEKDKVNSMGELPKEVDLAFIFHDWTKDYS
LFWKELATKHVPKLNKQGTRVIRTIPWRFLAGGDNSGIAEDTSKYPNTPEGNKALAKAIVDE
YVYKYNLDGLDVDVEHDSIPKVDKKEDTAGVERSIQVFEEIGKLIGPKGVDKSRLFIMDSTY
MADKNPLIERGAPYINLLLVQVYGSQGEKGGWEPVSNRPEKTMEERWQGYSKYIRPEQYM
IGFSFYEENAQEGNLWYDINSRKDEDKANGINTDITGTRAERYARWQPKTGGVKGGIFSYAI
DRDGVAHQPKKYAKQKEFKDATDNIFHSDYSVSKALKTVMLKDKSYDLIDEKDFPDKALRE
AVMAQVGTRKGDLERFNGTLRLDNPAIQSLEGLNKFKKLAQLDLIGLSRITKLDRSVLPANM
KPGKDTLETVLETYKKDNKEEPATIPPVSLKVSGLTGLKELDLSGFDRETLAGLDAATLTSLE
KVDISGNKLDLAPGTENRQIFDTMLSTISNHVGSNEQTVKFDKQKPTGHYPDTYGKTSLRLP
VANEKVDLQSQLLFGTVTNQGTLINSEADYKAYQNHKIAGRSFVDSNYHYNNFKVSYENYT
VKVTDSTLGTTTDKTLATDKEETYKVDFFSPADKTKAVHTAKVIVGDEKTMMVNLAEGATVI
GGSADPVNARKVFDGQLGSETDNISLGWDSKQSIIFKLKEDGLIKHWRFFNDSARNPETTN
KPIQEASLQIFNIKDYNLDNLLENPNKFDDEKYWITVDTYSAQGERATAFSNTLNNITSKYWR
VVFDTKGDRYSSPVVPELQILGYPLPNADTIMKTVTTAKELSQQKDKFSQKMLDELKIKEMA
LETSLNSKIFDVTAINANAGVLKDCIEKRQLLKKHHHHHHHHHH
SEQ ID NO. 7:
FQVLKSLGKLAMGSDSQSVSSSSTQDPHRGRQTLGSLRGLAKAKPEASFQVWNKDSSSK
NLIPRLQKIWKNYLSMNKYKVSYKGPGPGIKFSAEALRCHLRDHVNVSMVEVTDFPFNTSE
WEGYLPKESIRTKAGPWGRCAVVSSAGSLKSSQLGREIDDHDAVLRFNGAPTANFQQDVG
TKTTIRLMNSQLVTTEKRFLKDSLYNEGILIVWDPSVYHSDIPKWYQNPDYNFFNNYKTYRK
LHPNQPFYILKPQMPWELWDILQEISPEEIQPNPPSSGMLGIIIMMTLCDQVDIYEFLPSKRKT
DVCYYYQKFFDSACTMGAYHPLLYEKNLVKHLNQGTDEDIYLLGKATLPGFRTIHC
SEQ ID NO. 8:
RDLSRLPQLVGVSTPLQGGSNSAAAIGQSSGELRTGGARPPPPLGASSQPRPGGDSSPVV
DSGPGPASNLTSVPVPHTTALSLPACPEESPLLVGPMLIEFNMPVDLELVAKQNPNVKMGG
RYAPRDCVSPHKVAIIIPFRNRQEHLKYWLYYLHPVLQRQQLDYGIYVINQAGDTIFNRAKLL
NVGFQEALKDYDYTCFVFSDVDLIPMNDHNAYRCFSQPRHISVAMDKFGFSLPYVQYFGG
VSALSKQQFLTINGFPNNYWGWGGEDDDIFNRLVFRGMSISRPNAVVGRCRMIRHSRDKK
NEPNPQRFDRIAHTKETMLSDGLNSLTYQVLDVQRYPLYTQITVDIGTPS
SEQ ID NO. 9:
EEKTVQVQKGLPSIDSLHYLSENSKKEFKEELSKAGQESQKVKEILAKAQQADKQAQELAK
MKIPEKIPMKPLHGPLYGGYFRTWHDKTSDPTEKDKVNSMGELPKEVDLAFIFHDWTKDYS
LFWKELATKHVPKLNKQGTRVIRTIPWRFLAGGDNSGIAEDTSKYPNTPEGNKALAKAIVDE
YVYKYNLDGLDVDVEHDSIPKVDKKEDTAGVERSIQVFEEIGKLIGPKGVDKSRLFIMDSTY
MADKNPLIERGAPYINLLLVQVYGSQGEKGGWEPVSNRPEKTMEERWQGYSKYIRPEQYM
IGFSFYEENAQEGNLWYDINSRKDEDKANGINTDITGTRAERYARWQPKTGGVKGGIFSYAI
DRDGVAHQPKKYAKQKEFKDATDNIFHSDYSVSKALKTVMLKDKSYDLIDEKDFPDKALRE
AVMAQVGTRKGDLERFNGTLRLDNPAIQSLEGLNKFKKLAQLDLIGLSRITKLDRSVLPANM
KPGKDTLETVLETYKKDNKEEPATIPPVSLKVSGLTGLKELDLSGFDRETLAGLDAATLTSLE
KVDISGNKLDLAPGTENRQIFDTMLSTISNHVGSNEQTVKFDKQKPTGHYPDTYGKTSLRLP
VANEKVDLQSQLLFGTVTNQGTLINSEADYKAYQNHKIAGRSFVDSNYHYNNFKVSYENYT
VKVTDSTLGTTTDKTLATDKEETYKVDFFSPADKTKAVHTAKVIVGDEKTMMVNLAEGATVI
GGSADPVNARKVFDGQLGSETDNISLGWDSKQSIIFKLKEDGLIKHWRFFNDSARNPETTN
KPIQEASLQIFNIKDYNLDNLLENPNKFDDEKYWITVDTYSAQGERATAFSNTLNNITSKYWR
VVFDTKGDRYSSPVVPELQILGYPLPNADTIMKTVTTAKELSQQKDKFSQKMLDELKIKEMA
LETSLNSKIFDVTAINANAGVLKDCIEKRQLLKK

1. A glycoconjugate having the formula: 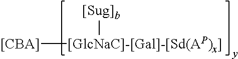
wherein:
CBA is a Cell Binding Agent;
GlcNAc is a N-acetyl-glucosamine moiety;
Sug is a sugar moiety, wherein b=1 or 0;
Gal is a galactose moiety;
Sd(A^{P})ₓ is a sugar derivative comprising x conjugated payloads A, wherein x=1, 2, 3, or 4;
wherein y=1 to 20; and
wherein the payload A^{P} is, comprises, or releases upon metabolism, a PBD compound such as a PBD dimer;
optionally wherein GlcNAc, Gal, Sug, and/or Sd(A^{P})ₓ are D enantiomers.

2. The glycoconjugate of claim 1, wherein Sd(A^{P})x is a sialic acid derivative.

3. The glycoconjugate of claim 2, wherein the sialic acid derivative has the formula: 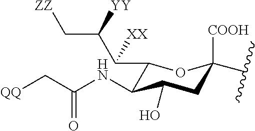
wherein:
QQ is hydrogen or a conjugated payload;
ZZ is hydroxyl or a conjugated payload;
YY is hydroxyl or a conjugated payload; and/or
XX is hydroxyl or a conjugated payload;
and wherein at least one of QQ, ZZ, YY, and XX is a conjugated payload.

4. The glycoconjugate of any preceding claim having the the formula: 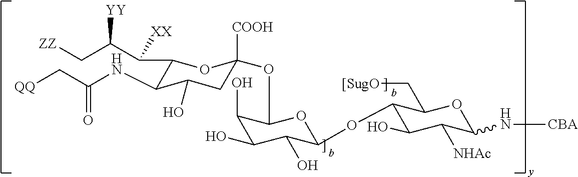
wherein:
QQ is hydrogen or a conjugated payload;
ZZ is hydroxyl or a conjugated payload;
YY is hydroxyl or a conjugated payload; and/or
XX is hydroxyl or a conjugated payload;
and wherein at least one of QQ, ZZ, YY, and XX is a conjugated payload

5. The glycoconjugate of any one of claims 1 to 4 having the the formula: 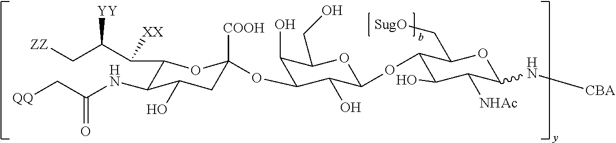
wherein:
QQ is hydrogen or a conjugated payload;
ZZ is hydroxyl or a conjugated payload;
YY is hydroxyl or a conjugated payload; and/or
XX is hydroxyl or a conjugated payload;
and wherein at least one of QQ, ZZ, YY, and XX is a conjugated payload.

6. The glycoconjugate of any preceding claim, wherein Sug is a fucose moiety.

7. The glycoconjugate of claim 6, wherein the fucose moiety has the structure:

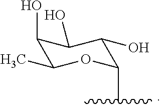 [SMILES: *C1OC(C)C(O)C(O)C1O]

8. The glycoconjugate of any one of claims 3 to 7, having a conjugated payload at position QQ or ZZ, wherein x=1.

9. The glycoconjugate of any preceding claim having a conjugated payload at each of positions QQ and ZZ, wherein x=2;
optionally wherein the payload at each of QQ and ZZ is the same.

10. The glycoconjugate of any preceding claim, wherein y=2.

11. The glycoconjugate of any preceding claim, wherein the CBA is a Fc fusion protein or an antibody.

12. The glycoconjugate of claim 11, wherein the GlcNAc moiety is conjugated to the Fc fusion protein or antibody at the asparagine 297 (Asn297) residue according to the EU index as set forth in Kabat.

13. The glycoconjugate of any preceding claim, wherein the payload is, comprises, or releases upon metabolism a PBD compound selected from the group consisting of:

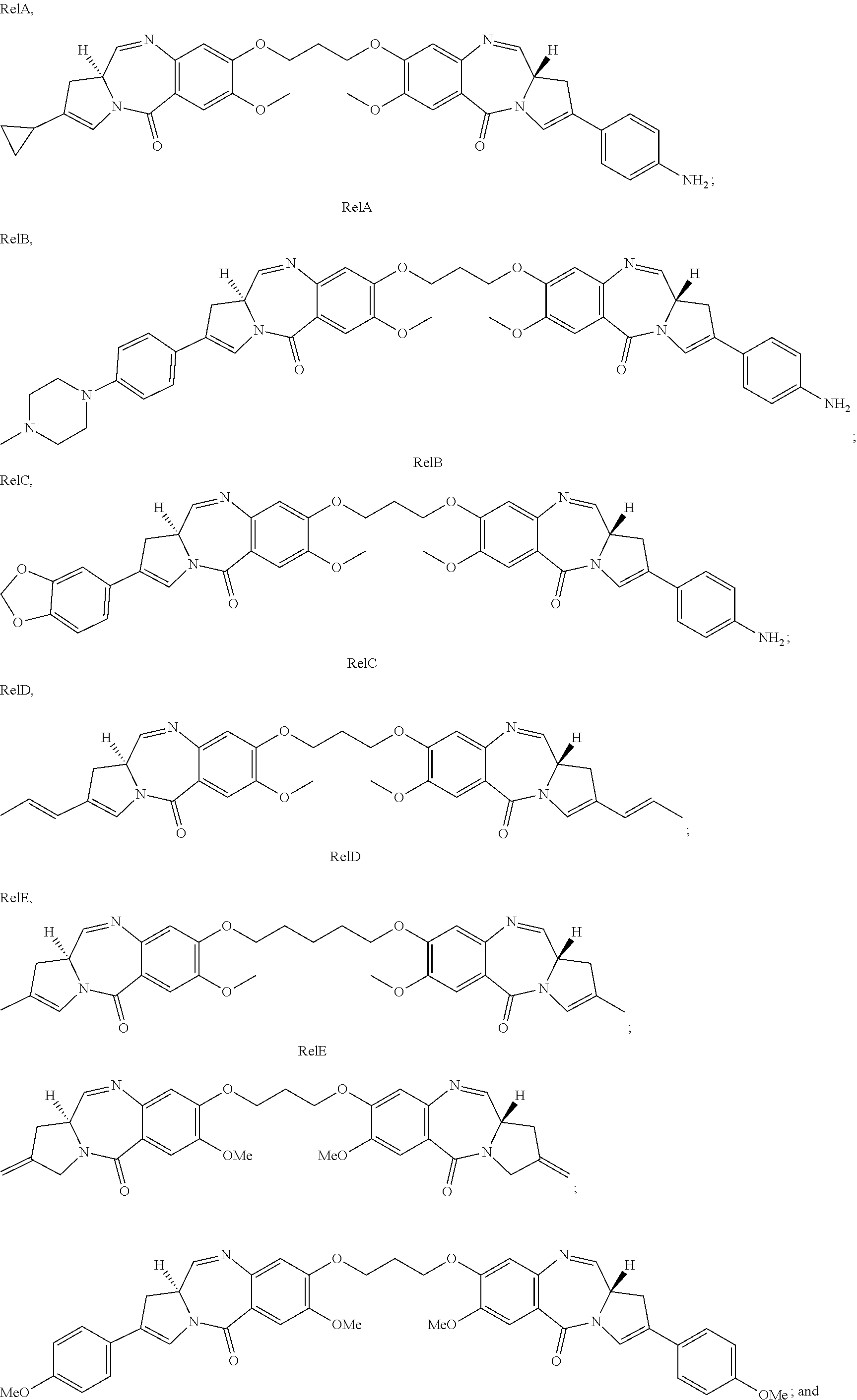

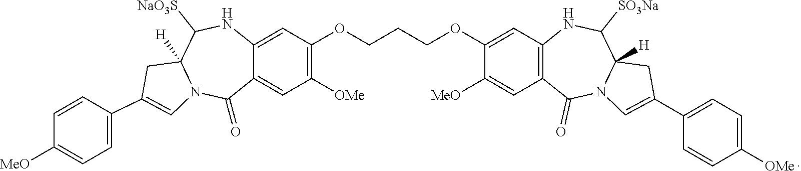

14. The glycoconjugate of any one of claims 1 to 13, wherein the conjugated payload, A^{P}, is a drug-linker comprising a drug moiety conjugated to the cell-binding agent via a linker moiety, the glycoconjugate having the formula:

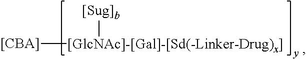

15. The glycoconjugate of claim 14, wherein each linker independently is a linker of formula Z1 or Z2: 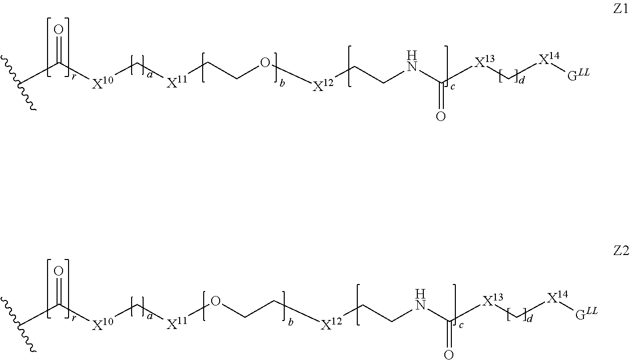
wherein r=0 or 1, a=0 to 5, b=0 to 16, c=0 or 1, d=0 to 5, G^{LL} is a linking moiety through which the linker is bound to the Sd moiety, the wavy line indicates where the linker is bound to the drug moiety, and one of X¹⁰, X¹¹, X¹², X¹³ and X¹⁴ may be selected from: 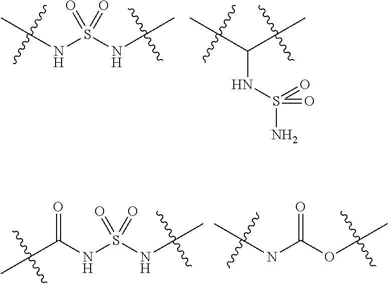 [SMILES: *NS(=O)(=O)N* ; *C(*)NS(N)(=O)=O ; *NS(=O)(=O)NC(*)=O ; *NC(=O)O*]
the remainder being a single bond.

16. The glycoconjugate of claim 15, wherein G^{LL} is selected from:

| | |
|---|---|
| (G^{LL8-1}) | 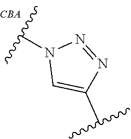 [SMILES: *c1cn(*)nn1] |
| (G^{LL8-2}) | 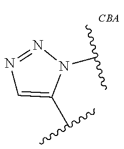 [SMILES: *c1cnnn1*] |
| (G^{LL9-1}) | 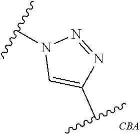 [SMILES: *c1cn(*)nn1] |
| (G^{LL9-2}) | 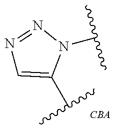 [SMILES: *c1cnnn1*] |
| G^{LL10} | 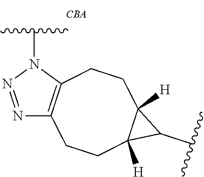 [SMILES: *C1[C@H]2CCc3c(nnn3*)CC[C@@H]12] |
| G^{LL11} | 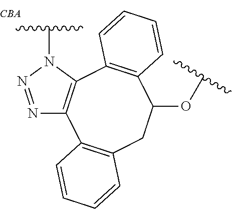 [SMILES: *OC1Cc2ccccc2-c2nnn(*)c2-c2ccccc21] |
| G^{LL12} | 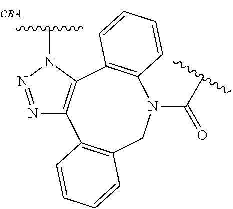 [SMILES: *C(=O)N1Cc2ccccc2-c2nnn(*)c2-c2ccccc21] |
| G^{LL13} | 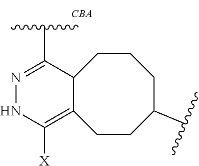 |
| G^{LL14} | 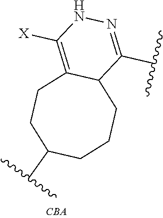 |

where CBA indicates where the group is bound to Sd(A^{P})ₓ.

17. A method for the preparation of the glycoconjugate of any one of claims 1 to 16, the method comprising the steps of:
(i) providing a glycosylated cell-binding agent;
(ii) reacting the glycosylated cell-binding agent of (i) with a compound of the formula payload-G^{L}, wherein the payload is as defined in any one of claims 1 to 16 and G^{L} is linker group reactive with the functional group of the glycosylated cell-binding agent

18. The method of claim 17, wherein G^{L} is selected from the group consisting of:

| | |
|---|---|
| (G^{L8}) | 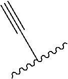 [SMILES: *C#C] |
| (G^{L9}) | 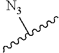 [SMILES: *N=[N+]=[N-]] |
| (G^{L10}) | 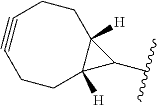 [SMILES: *C1[C@H]2CCC#CCC[C@@H]12] |
| (G^{L11}) | 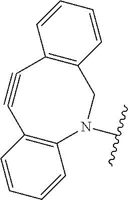 [SMILES: *N1Cc2ccccc2C#Cc2ccccc21] |
| (G^{L12}) | 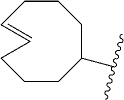 [SMILES: *C1CCC/C=C/CCC1] |
| (G^{L13}) | 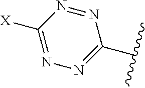 |

19. The method of either one of claim 17 or 18, wherein the provided glycosylated cell-binding agent is a glycosylated cell-binding agent having the formula: 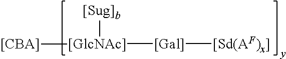
wherein:
CBA is a Cell Binding Agent;
GlcNAc is a N-acetyl-glucosamine moiety;
Sug is a sugar moiety, wherein b=1 or 0;
Gal is a galactose moiety;
Sd(A^{F})ₓ is a sugar derivative comprising x functional groups A^{F}, wherein A^{F} is independently selected from the group consisting of an azido group, an alkynyl group, and a keto group, and wherein x=1, 2, 3, or 4; and
wherein y=1 to 20.

20. The method of claim 19, wherein Sd(A^{F})x is a sialic acid derivative having the formula: 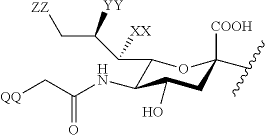
wherein:
QQ is hydrogen or a functional group A^{F};
ZZ is hydroxyl or a functional group A^{F};
YY is hydroxyl or a functional group A^{F}; and/or
XX is hydroxyl or a functional group A^{F};
and wherein at least one of QQ, ZZ, YY, and XX is a functional group A^{F}.

21. The method of any one of claims 17 to 20, wherein the glycosylated cell-binding agent is provided by a method comprising the steps of:
(i) providing a Sd(A^{F})ₓ acceptor having the formula: 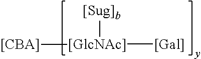
wherein CBA, GlcNAc, Sug, b, Gal, and y are defined as in of any one of claims 17 to 20; and
(ii) contacting the Sd(A^{F})ₓ acceptor with a compound of the formula Sd(A^{F})ₓ-P* in the presence of a glycosyltransferase, wherein:
Sd(A^{F})ₓ is as defined in any one of claims 17 to 20; and
P* is a nucleoside phosphate moiety.

22. The method of any one of claims 88 to 92, wherein the nucleoside element of the nucleoside phosphate moiety is selected from the group consisting of: UDP, GDP, TDP, CDP, and CMP.

23. The method of either one of claim 21 or 22, wherein the Sd(A^{F})ₓ acceptor is provided by a process comprising the steps of:
a) providing a Gal acceptor having the formula: 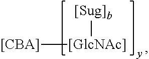
wherein CBA, GlcNAc, Sug, b, and y are defined as in claim 1; and
b) contacting the Gal acceptor with a compound of the formula Gal-P* in the presence of a galactosyltransferase, wherein Gal is a Galactose moiety and P* is a nucleoside phosphate moiety;
optionally wherein,
c) the Gal acceptor is produced by contacting with a glycosidase a oligoglycosylated cell-binding agent having the formula: 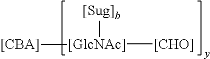
wherein CHO is a carbohydrate moiety.

24. The method of any one of claims 21 to 23, wherein the Sd(A^{F})ₓ acceptor has only two terminal galactose moieties.

25. The method of any one of claims 21 to 24, wherein the glycosyltransferase is a sialyltransferase.

26. The method of claim 25, wherein the sialyltransferase is a polypeptide having sialyltransferase activity and comprising a sequence having at least 70% sequence identity SEQ ID NO. 1, SEQ ID NO. 4, or SEQ ID NO. 7.

27. The method of any one of claims 21 to 26, wherein the glycosidase is an endoglycosidase.

28. The method of claim 27, wherein the endoglycosidase is a polypeptide having endoglycosidase activity and comprising a sequence having at least 70% sequence identity SEQ ID NO. 3, SEQ ID NO. 6, or SEQ ID NO. 9.

29. The method of any one of claims 21 to 28, wherein the galactosyltransferase is a polypeptide having galactosyltransferase activity and comprising a sequence having at least 70% sequence identity SEQ ID NO. 2, SEQ ID NO. 5, or SEQ ID NO. 8.

30. A glycoconjugate of any one of claims 1 to 16 for use in a method of treatment.

31. A glycoconjugate of any one of claims 1 to 16 for use in a method of treating a proliferative disorder.

32. A method of treating a proliferative disorder, the method comprising administering an effective amount of a glycoconjugate of any one of claims 1 to 16 to a subject.

33. Use of a glycoconjugate of any one of claims 1 to 16 in the manufacture of a medicament for the treatment of a proliferative disorder.

34. The glycoconjugate, method, or use of any one of claims 31 to 33, wherein the proliferative disorder is cancer.

35. The glycoconjugate, method, or use of claim 34, wherein the cancer is selected from the group consisting of: histocytoma, glioma, astrocyoma, neuroblastoma, osteoma, lung cancer, gastrointestinal cancer, bowel cancer, colon cancer, breast carinoma, ovarian carcinoma, prostate cancer, testicular cancer, liver cancer, kidney cancer, bladder cancer, pancreas cancer, renal cancer, brain cancer, sarcoma, liposarcoma, osteosarcoma, Kaposi's sarcoma, melanoma, lymphomas, myeloma, and leukemias.

36. A method for the preparation of a glycoconjugate, the method comprising the steps of:
a) providing an oligoglycosylated cell-binding agent having the formula: 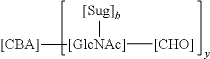
wherein CBA is a Cell Binding Agent;
GlcNAc is a N-acetyl-glucosamine moiety;
Sug is a sugar moiety, wherein b=1 or 0;
y=1 to 20; and
CHO is a carbohydrate moiety;
b) contacting the oligoglycosylated cell-binding agent with a glycosidase to produce a Gal acceptor having the formula: 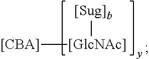
c) contacting the Gal acceptor with a compound of the formula Gal-P* in the presence of a galactosyltransferase to produce a Sd(A^{F})ₓ acceptor having the formula: 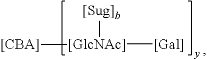
wherein Gal is a galactose moiety, and
P* is a nucleoside phosphate moiety;
d) contacting the Sd(A^{F})ₓ acceptor with a compound of the formula Sd(A^{F})ₓ-P* in the presence of a glycosyltransferase to produce a glycosylated cell-binding agent having the formula: 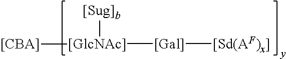
wherein:
Sd(A^{F})ₓ is a sugar derivative comprising x functional groups A^{F}, wherein x=1, 2, 3, or 4;
e) reacting the glycosylated cell-binding agent with a compound of the formula payload-G^{L},
wherein the payload is as according to any one of statements 39 to 52 and G^{L} is a linker group reactive with the functional group A^{F} of the glycosylated cell-binding agent,
to produce a glycoconjugate having the formula: 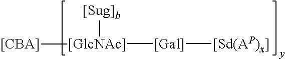
wherein:
Sd(A^{P})ₓ is a sugar derivative comprising x conjugated payloads A^{P}, wherein x=1, 2, 3, or 4;
wherein steps (b), (c), and (d) are performed in the same reaction volume.

37. The method according to claim 36, wherein the Gal acceptor product of step (b) is not purified from the reaction volume before it is contacted with the galactosyltransferase of step (c); and
wherein the Sd(A^{F})ₓ acceptor product of step (c) is not purified from the reaction volume before it is contacted with the glycosyltransferase of step (d).

38. The method according to either one of claim 36 or 37, wherein steps (b), (c), and (d) are performed at the same time.

39. The method according to any one of claims 36 to 38, wherein steps (b), (c), and (d) comprise an incubation of between 24 and 48 hours, such as about 36 hours.

40. The method according to any one of claims 36 to 39, wherein the method further comprises an additional step (d′) between steps (d) and (e), wherein step (d′) comprises the addition of further Sd(A^{F})ₓ-P* and/or glycosyltransferase.

41. The method according to claim 40, wherein the further Sd(A^{F})ₓ-P* and/or glycosyltransferase are added on completion of the incubation of step (d).
